(19) United States
(12) Patent Application Publication
(10) Pub. No.: US 2024/0335252 A1
(43) Pub. Date: Oct. 10, 2024

(54) APPARATUS AND METHODS FOR TREATING TEETH

(71) Applicant: SONENDO, INC., Laguna Hills, CA (US)

(72) Inventors: Bjarne Bergheim, Mission Viejo, CA (US); Kevin Wei-Kung Yein, Tustin, CA (US); Jeffrey William Schultz, Anaheim, CA (US); Ruijin Tian, Irvine, CA (US)

(21) Appl. No.: 18/627,341

(22) Filed: Apr. 4, 2024

Related U.S. Application Data
(60) Provisional application No. 63/457,656, filed on Apr. 6, 2023.
(60) Provisional application No. 63/457,701, filed on Apr. 6, 2023.

(51) Int. Cl. A61C 1/02; A61B 1/24; A61C 19/06

(52) U.S. Cl. CPC A61C 1/02; A61B 1/24; A61C 19/066

(57) ABSTRACT

Methods and devices for treating dental caries includes one or more of assessing, disinfecting, neutralizing, remineralizing, and sealing of a carious region of a tooth. The method of treating dental caries includes assessing the condition of the tooth before treatment. A treatment instrument disclosed herein can be used to disinfect, neutralize, remineralize, and/or seal the carious region of the tooth. The method of treating dental caries can further include assessing the conditions of the tooth after remineralizing and again after sealing.

### CROSS-REFERENCE TO RELATED APPLICATIONS

This application claims priority to U.S. Provisional Patent Application No. 63/457,656, filed Apr. 6, 2023; and U.S. Provisional Patent Application No. 63/457,701, filed Apr. 6, 2023, the entire contents of each of which are incorporated by reference herein in their entirety and for all purposes. Any and all applications for which a foreign or domestic priority claim is identified in the Application Data Sheet as filed with the present application are hereby incorporated by reference under 37 CFR 1.57 for all purposes and for all that they contain.

### BACKGROUND

### Field of the Invention

The field relates to an apparatus for and method for treating teeth.

### Description of the Related Art

In conventional dental and endodontic procedures, mechanical instruments such as drills, files, brushes, etc. are used to clean unhealthy material from a tooth. For example, dentists often use drills to mechanically break up carious regions (e.g., cavities) on a surface of the tooth. Such procedures are often painful for the patient and frequently do not remove all the diseased material. Furthermore, in conventional root canal treatments, an opening is drilled through the crown of a diseased tooth, and endodontic files are inserted into the root canal system to open the canal spaces and remove organic material therein. The root canal is then filled with solid matter such as gutta percha or a flowable obturation material, and the tooth is restored. However, this procedure will not remove all organic material from the canal spaces, which can lead to post-procedure complications such as infection. In addition, motion of the endodontic file and/or other sources of positive pressure may force organic material through an apical opening into periapical tissues. In some cases, an end of the endodontic file itself may pass through the apical opening. Such events may result in trauma to the soft tissue near the apical opening and lead to post-procedure complications. Accordingly, there is a continuing need for improved dental and endodontic treatments.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices, components and methods for treating teeth.

In one embodiment, an apparatus for treating a tooth is disclosed. The apparatus can include a proximal chamber, a distal chamber disposed distal the proximal chamber and in fluid communication with the proximal chamber by way of a transition opening, the distal chamber having an access opening disposed apart from and distal to the transition opening, the access opening to provide fluid communication between a treatment region of the tooth and the distal chamber, a liquid supply port disposed to direct a liquid stream into the proximal chamber and over at least a portion of the transition opening, and an impingement member arranged within a path of the liquid stream, the impingement member having one or more surfaces positioned to redirect at least a portion of the liquid stream across at least a portion of the transition opening.

In some embodiments, the impingement member can a lateral width that is no wider that a lateral dimension of the transition opening. The distal chamber can have a cross-section area at least substantially equal to an area of the transition opening. The apparatus can include one or more flow disruptors positioned within the proximal chamber. The one or more flow disruptors can include one or more curved or angled protrusions extending from an inner surface of the proximal chamber. The liquid supply port and the impingement member can be arranged relative to each other to create a turbulent flow of liquid within the treatment region over a course of a treatment procedure. The proximal chamber can have a first interior surface geometry and the distal chamber can have a second interior surface geometry different than the first interior surface geometry. The apparatus can include a non-uniform transition between the proximal chamber and the distal chamber. A ratio of a volume of the proximal chamber to a volume of the distal chamber can be between 7:4 and 15:2. A ratio of a volume of the proximal chamber to a circumference of the transition opening can be between 1 in³:150 in and 1 in³:20 in. The liquid stream can include a jet and a ratio of a jet distance to a volume of the proximal chamber can be between 10 in:1 in³ and 50 in:1 in³. The liquid stream can include a jet and a ratio of a jet distance to a jet height can be between 2:1 and 13:2. The apparatus can include a suction port exposed to the proximal chamber. The suction port can be disposed along an upper wall of the proximal chamber. The apparatus can include an outlet line connected to the suction port. The apparatus can include a vent exposed to ambient air, the vent in fluid communication with the outlet line and being positioned along the outlet line at a location downstream of the suction port. A treatment fluid within the proximal chamber and the distal chamber can be a substantially degassed treatment fluid. The liquid supply port can be disposed to direct the liquid stream to generate pressure waves in a treatment fluid within the proximal chamber and the distal chamber, the generated pressure waves having a broadband power spectrum. The liquid supply port can be disposed to direct the liquid stream to impinge on the one or more surfaces of the impingement member at a contact point superior to a vertical center of the impingement member. The one or more surfaces of the impingement member can be shaped to redirect at least a portion of the liquid stream within the proximal chamber from a position inferior to the vertical center of the impingement member. The liquid supply port can be disposed to direct the liquid stream to impinge on the one or more surfaces of the impingement member at a contact point lateral to a horizontal center of the impingement member. The one or more surfaces of the impingement member can be shaped to redirect at least a portion of the liquid stream within the proximal chamber from a position lateral to the horizontal center of the impingement member on a side of the impingement member opposite the contact point. The liquid stream can be a liquid jet, wherein the one or more surfaces of the impingement member are shaped to redirect at least a portion of the liquid jet across at least a portion of the transition opening in the form of a second liquid jet. The liquid supply port can be disposed to direct the liquid stream to impinge on the one or more surfaces of the impingement member at a contact point inferior to a vertical center of the impingement member. The one or more surfaces of the impingement member are shaped to redirect at least a portion of the liquid stream within the proximal chamber from a position superior to the vertical center of the impingement member.

In another embodiment, an apparatus for treating a tooth during is provided. The apparatus can include, a proximal chamber, a distal chamber disposed distal the proximal chamber and in fluid communication with the proximal chamber by way of a transition opening, the distal chamber having an access opening disposed apart from and distal to the transition opening, the access opening to provide fluid communication between the distal chamber and a treatment region of the tooth, and a liquid supply port disposed to direct a liquid stream into the proximal chamber and over at least a portion of the transition opening to impinge on an impingement member, wherein the proximal chamber, the liquid supply port, the distal chamber, and the impingement member are arranged relative to one another in a manner that creates a turbulent flow of liquid within the treatment region over a course of a treatment procedure.

In some embodiments, the apparatus can include one or more flow disruptors positioned within the proximal chamber. The one or more flow disruptors can include one or more curved or angled protrusions extending from an inner surface of the proximal chamber. The proximal chamber can have a first interior surface geometry and the distal chamber can have a second interior surface geometry different than the first interior surface geometry. The apparatus can include a non-uniform transition between the proximal chamber and the distal chamber. A ratio of a volume of the proximal chamber to a volume of the distal chamber can be between 7:4 and 15:2. A ratio of a volume of the proximal chamber to a circumference of the transition opening can be between 1 in³:150 in and 1 in³:20 in. The liquid stream can be a jet and a ratio of a jet distance to a volume of the proximal chamber can be between 10 in:1 in³ and 50 in:1 in³. The liquid stream can be a jet and a ratio of a jet distance to a jet height can be between 2:1 and 13:2. The apparatus can include a suction port exposed to the proximal chamber. The suction port can be disposed along an upper wall of the proximal chamber. The apparatus can include an outlet line connected to the suction port. The apparatus can include a vent exposed to ambient air, the vent in fluid communication with the outlet line and being positioned along the outlet line at a location downstream of the suction port. A treatment fluid within the proximal chamber and the distal chamber can include a substantially degassed treatment fluid. The liquid supply port can be disposed to direct the liquid stream to generate pressure waves in a treatment fluid within the proximal chamber and the distal chamber, the generated pressure waves having a broadband power spectrum. The liquid supply port can be disposed to direct the liquid stream to impinge on an impingement surface of the impingement member at a contact point superior to a vertical center of the impingement surface. The impingement surface can be shaped to redirect at least a portion of the liquid stream within the proximal chamber from a position inferior to the vertical center of the impingement surface. The liquid supply port can be disposed to direct the liquid stream to impinge on an impingement surface of the impingement member at a contact point lateral to a horizontal center of the impingement member. The impingement surface can be shaped to redirect at least a portion of the liquid jet within the proximal chamber from a position lateral to the horizontal center of the impingement surface on a side of the impingement surface opposite the contact point. The liquid stream can include a liquid jet, wherein an impingement surface of the impingement member is shaped to redirect at least a portion of the liquid jet into the proximal chamber in the form of a second liquid jet. The liquid supply port can be disposed to direct the liquid stream to impinge on an impingement surface of the impingement member at a contact point inferior to a vertical center of the impingement surface. The impingement surface can be shaped to redirect at least a portion of the liquid stream within the proximal chamber from a position superior to the vertical center of the impingement surface.

In another embodiment, an apparatus for treating a tooth is provided. The apparatus can include a proximal chamber, a distal chamber disposed distal the proximal chamber and in fluid communication with the proximal chamber by way of a transition opening, the distal chamber having an access opening disposed apart from and distal to the transition opening, the access opening to provide fluid communication between the distal chamber and a treatment region of the tooth, and a liquid supply port disposed to direct a liquid stream into the proximal chamber and over at least a portion of the transition opening to impinge on an impingement member, the impingement member having one or more surfaces positioned to redirect at least a portion of the liquid stream over at least a portion of the transition opening to produce toroidal flow in the distal chamber.

In some embodiments, the apparatus can include one or more flow disruptors positioned within the proximal chamber. The one or more flow disruptors can include one or more curved or angled protrusions extending from an inner surface of the proximal chamber. The liquid supply port and the impingement member can be arranged relative to each other to create a turbulent flow of liquid within the treatment region over a course of a treatment procedure. The proximal chamber can have a first interior surface geometry and the distal chamber can have a second interior surface geometry different than the first interior surface geometry. The apparatus can include a non-uniform transition between the proximal chamber and the distal chamber. A ratio of a volume of the proximal chamber to a volume of the distal chamber can be between 7:4 and 15:2. A ratio of a volume of the proximal chamber to a circumference of the transition opening can be between 1 in³:150 in and 1 in³:20 in. The liquid stream can include a jet and a ratio of a jet distance to a volume of the proximal chamber can be between 10 in:1 in³ and 50 in:1 in³. The liquid stream can be a jet and a ratio of a jet distance to a jet height can be between 2:1 and 13:2. The apparatus can be a suction port exposed to the proximal chamber. The suction port can be disposed along an upper wall of the proximal chamber. The apparatus can include an outlet line connected to the suction port. The apparatus can include a vent exposed to ambient air, the vent in fluid communication with the outlet line and being positioned along the outlet line at a location downstream of the suction port. A treatment fluid within the proximal chamber and the distal chamber can include a substantially degassed treatment fluid. The liquid supply port can be disposed to direct the liquid stream to generate pressure waves in a treatment fluid within the proximal chamber and the distal chamber, the generated pressure waves having a broadband power spectrum. The liquid supply port can be disposed to direct the liquid stream to impinge on the one or more surfaces of the impingement member at a contact point superior to a vertical center of the impingement member. The one or more surfaces of the impingement member can be shaped to redirect at least a portion of the liquid stream within the proximal chamber from a position inferior to the vertical center of the impingement member. The liquid supply port can be disposed to direct the liquid stream to impinge on the one or more surfaces of the impingement member at a contact point lateral to a horizontal center of the impingement member. The one or more surfaces of the impingement member can be shaped to redirect at least a portion of the liquid stream within the proximal chamber from a position lateral to the horizontal center of the impingement member on a side of the impingement member opposite the contact point. The liquid stream can include a liquid jet, wherein the one or more surfaces of the impingement member can be shaped to redirect at least a portion of the liquid jet across at least a portion of the transition opening in the form of a second liquid jet. The liquid supply port can be disposed to direct the liquid stream to impinge on the one or more surfaces of the impingement member at a contact point inferior to a vertical center of the impingement member. The one or more surfaces of the impingement member can be shaped to redirect at least a portion of the liquid stream within the proximal chamber from a position superior to the vertical center of the impingement member.

In another embodiment, an apparatus for treating a tooth is provided. The apparatus can include a proximal chamber having a first interior surface geometry, a distal chamber disposed distal the proximal chamber and in fluid communication with the proximal chamber by way of a transition opening, the distal chamber having an access opening disposed apart from and distal to the transition opening, the access opening to provide fluid communication between the distal chamber and a treatment region of the tooth, the distal chamber having a second interior surface geometry that is different than the first interior surface geometry, and a liquid supply port disposed to direct a liquid stream into the proximal chamber and over at least a portion of the access opening.

In some embodiments, the apparatus can include one or more flow disruptors positioned within the proximal chamber. The one or more flow disruptors can include one or more curved or angled protrusions extending from an inner surface of the proximal chamber. The liquid supply port and an impingement member can be arranged relative to each other to create a turbulent flow of liquid within the treatment region over a course of a treatment procedure. The apparatus can include a non-uniform transition between the proximal chamber and the distal chamber. A ratio of a volume of the proximal chamber to a volume of the distal chamber can be between 7:4 and 15:2. A ratio of a volume of the proximal chamber to a circumference of the transition opening can be between 1 in³:150 in and 1 in³:20 in. The liquid stream can include a jet and a ratio of a jet distance to a volume of the proximal chamber can be between 10 in:1 in³ and 50 in:1 in³. The liquid stream can include a jet and a ratio of a jet distance to a jet height can be between 2:1 and 13:2. The apparatus can include a suction port exposed to the proximal chamber. The suction port can be disposed along an upper wall of the proximal chamber. The apparatus can include an outlet line connected to the suction port. The apparatus can include a vent exposed to ambient air, the vent in fluid communication with the outlet line and being positioned along the outlet line at a location downstream of the suction port. A treatment fluid within the proximal chamber and the distal chamber can include a substantially degassed treatment fluid. The liquid supply port can be disposed to direct the liquid stream to generate pressure waves in a treatment fluid within the proximal chamber and the distal chamber, the generated pressure waves having a broadband power spectrum. The liquid supply port can be disposed to direct the liquid stream to impinge on an impingement surface of an impingement member at a contact point superior to a vertical center of the impingement surface. The impingement surface can be shaped to redirect at least a portion of the liquid stream within the proximal chamber from a position inferior to the vertical center of the impingement surface. The liquid supply port can be disposed to direct the liquid stream to impinge on an impingement surface of an impingement member at a contact point lateral to a horizontal center of the impingement member. The impingement surface can be shaped to redirect at least a portion of the liquid jet within the proximal chamber from a position lateral to the horizontal center of the impingement surface on a side of the impingement surface opposite the contact point. The liquid stream can include a liquid jet, wherein the liquid supply port can be disposed to direct the liquid jet to impinge on an impingement surface of an impingement member, wherein the impingement surface can be shaped to redirect at least a portion of the liquid jet into the proximal chamber in the form of a second liquid jet. The liquid supply port can be disposed to direct the liquid stream to impinge on an impingement surface of an impingement member at a contact point inferior to a vertical center of the impingement surface. The impingement surface can be shaped to redirect at least a portion of the liquid stream within the proximal chamber from a position superior to the vertical center of the impingement surface.

In another embodiment, an apparatus for treating a tooth is provided. The apparatus can include a proximal chamber, a distal chamber disposed distal the proximal chamber and in fluid communication with the proximal chamber, the distal chamber having an access opening disposed apart from and distal the proximal chamber, the access opening to provide fluid communication between the distal chamber and a treatment region of the tooth, a liquid supply port disposed to direct a liquid stream across the proximal chamber, and a non-uniform transition region between the proximal chamber and the distal chamber.

In some embodiments, the non-uniform transition region can include a discontinuity providing a non-uniform or abrupt flow transition between the proximal and distal chambers. The discontinuity can be provided by a transition opening and differing interior surface geometries of the proximal chamber and the distal chamber. The non-uniform transition region can include asymmetric interior surfaces of one or more of the proximal chamber and the distal chamber. The non-uniform transition region can include one or more disruptive interior surfaces of one or more of the proximal chamber and the distal chamber. The apparatus can include a transition opening between the proximal chamber and the distal chamber, and an impingement ring, at least a portion of the impingement ring being recessed from the transition opening and at least a portion of the impingement ring extending over at least a portion of the transition opening to form the non-uniform transition region. The apparatus can include one or more flow disruptors positioned within the proximal chamber. The one or more flow disruptors can include one or more curved or angled protrusions extending from an inner surface of the proximal chamber. The liquid supply port and an impingement member can be arranged relative to each other to create a turbulent flow of liquid within the treatment region over a course of a treatment procedure. The proximal chamber can have a first interior surface geometry and the distal chamber can have a second interior surface geometry different than the first interior surface geometry. A ratio of a volume of the proximal chamber to a volume of the distal chamber can be between 7:4 and 15:2. The apparatus can include a transition opening between the proximal chamber and the distal chamber, wherein a ratio of a volume of the proximal chamber to a circumference of the transition opening can be between 1 in³:150 in and 1 in³:20 in. The liquid stream can include a jet and a ratio of a jet distance to a volume of the proximal chamber can be between 10 in:1 in³ and 50 in:1 in³. The liquid stream can include a jet and a ratio of a jet distance to a jet height can be between 2:1 and 13:2. The apparatus can include a suction port exposed to the proximal chamber. The suction port can be disposed along an upper wall of the proximal chamber. The apparatus can include an outlet line connected to the suction port. The apparatus can include a vent exposed to ambient air, the vent in fluid communication with the outlet line and being positioned along the outlet line at a location downstream of the suction port. A treatment fluid within the proximal chamber and the distal chamber can include a substantially degassed treatment fluid. The liquid supply port can be disposed to direct the liquid stream to generate pressure waves in a treatment fluid within the proximal chamber and the distal chamber, the generated pressure waves having a broadband power spectrum. The liquid supply port can be disposed to direct the liquid stream to impinge on an impingement surface of an impingement member at a contact point superior to a vertical center of the impingement surface. The impingement surface can be shaped to redirect at least a portion of the liquid stream within the proximal chamber from a position inferior to the vertical center of the impingement surface. The liquid supply port can be disposed to direct the liquid stream to impinge on an impingement surface of an impingement member at a contact point lateral to a horizontal center of the impingement member. The impingement surface can be shaped to redirect at least a portion of the liquid jet within the proximal chamber from a position lateral to the horizontal center of the impingement surface on a side of the impingement surface opposite the contact point. The liquid stream can include a liquid jet, wherein the liquid supply port can be disposed to direct the liquid jet to impinge on an impingement surface of an impingement member, wherein the impingement surface can be shaped to redirect at least a portion of the liquid jet into the proximal chamber in the form of a second liquid jet. The liquid supply port can be disposed to direct the liquid stream to impinge on an impingement surface of an impingement member at a contact point inferior to a vertical center of the impingement surface. The impingement surface can be shaped to redirect at least a portion of the liquid stream within the proximal chamber from a position superior to the vertical center of the impingement surface.

In another embodiment, an apparatus for treating a tooth is provided. The apparatus can include a proximal chamber, a distal chamber disposed distal the proximal chamber and in fluid communication with the proximal chamber by way of a transition opening, the distal chamber having an access opening disposed apart from and distal to the transition opening, the access opening to provided fluid communication between a treatment region of the tooth and the distal chamber, an impingement member including an impingement surface, and a liquid supply port disposed to direct a liquid jet to impinge on the impingement surface at a contact point superior to a vertical center of the impingement surface, wherein the impingement surface is shaped to redirect at least a portion of the liquid jet within the proximal chamber from a position inferior to the vertical center of the impingement surface.

In some embodiments, the liquid supply port can be disposed to direct the liquid jet to impinge on the impingement surface at the contact point lateral to a horizontal center of the impingement surface. The impingement surface can be shaped to redirect at least a portion of the liquid jet within the proximal chamber from a position lateral to the horizontal center of the impingement surface on a side of the impingement surface opposite the contact point. An angle between a vertical axis of the impingement surface and a radial line extending from a center point of the impingement surface through the contact point can be between −45° and 45°. The angle can be between −30° and 30°. The angle can be between −15° and 15°. The liquid jet can be disposed to impinge on the impingement surface at a contact point at a radial distance less than 0.63 inches from a center point of the impingement surface. The liquid jet can be disposed to impinge on the impingement surface at a contact point at a radial distance between 0.010 inches and 0.05 inches from the center point of the impingement surface. The liquid jet can be disposed to impinge on the impingement surface at the contact point at a radial distance between 1% and 49% of a diameter of the impingement surface. The liquid jet can be disposed to impinge on the impingement surface at the contact point at a radial distance between 5% and 45% of a diameter of the impingement surface. The liquid jet can be disposed to impinge on the impingement surface at the contact point at a radial distance between 8% and 40% of a diameter of the impingement surface. The liquid jet can be disposed to impinge on the impingement surface at the contact point at a radial distance between 15% and 25% of a diameter of the impingement surface. The liquid jet can be disposed to impinge on the impingement surface at the contact point at a radial distance between 20% and 40% of a diameter of the impingement surface. The impingement member can be angled downwardly towards the transition opening. A central axis of the impingement member can be angled inferiorly from an anterior-posterior axis of the proximal chamber by an angle between 0° and 10°. The central axis of the impingement member can be angled inferiorly from the anterior-posterior axis of the proximal chamber by an angle between 0° and 6°. The central axis of the impingement member can be angled inferiorly from the anterior-posterior axis of the proximal chamber by an angle between 0° and 3°. A central axis of the impingement member can be angled laterally relative to a superior-inferior axis of the proximal chamber. The liquid supply port can be disposed to direct the liquid jet along a jet axis angled superiorly to an anterior-posterior axis of the proximal chamber. The liquid supply port can be disposed to direct the liquid jet along the jet axis superiorly to the anterior-posterior axis of the proximal chamber by an angle between 0° and 10°. The liquid supply port can be disposed to direct the liquid jet along the jet axis superiorly to the anterior-posterior axis of the proximal chamber by an angle between 0° and 6°. The liquid supply port can be disposed to direct the liquid jet along the jet axis superiorly to the anterior-posterior axis of the proximal chamber by an angle between 0° and 4°. The liquid supply port can be disposed to direct the liquid jet along a jet axis angled laterally relative to a superior-inferior axis of the proximal chamber. The impingement surface can be shaped to redirect at least a portion of the liquid jet within the proximal chamber in the form of a second liquid jet. The impingement surface can be angled at the contact point to redirect at least a portion of the liquid jet within the proximal chamber in the form of a second liquid jet. The liquid jet can be disposed to impinge on the impingement surface at an angle relative to the impingement surface configured to cause the liquid jet to be redirected from the impingement surface in the form of a second liquid jet. The impingement surface can be hemispherical. The impingement surface can be concave. The liquid supply port and the impingement member can be arranged relative to each other to create a turbulent flow of liquid within the treatment region over a course of a treatment procedure. The apparatus can include a suction port exposed to the proximal chamber. The suction port can be disposed along an upper wall of the proximal chamber. The apparatus can include an outlet line connected to the suction port. The apparatus can include a vent exposed to ambient air, the vent in fluid communication with the outlet line and being positioned along the outlet line at a location downstream of the suction port. A treatment fluid within the proximal chamber and the distal chamber can include a substantially degassed treatment fluid. The liquid supply port can be disposed to direct the liquid jet to generate pressure waves in a treatment fluid within the proximal chamber and the distal chamber, the generated pressure waves having a broadband power spectrum.

In another embodiment, an apparatus for treating a tooth is provided. The apparatus can include a proximal chamber, a distal chamber disposed distal the proximal chamber and in fluid communication with the proximal chamber by way of a transition opening, the distal chamber having an access opening disposed apart from and distal to the transition opening, the access opening to provide fluid communication between a treatment region of the tooth and the distal chamber, a liquid supply port disposed to direct a liquid jet into the proximal chamber, and an impingement member arranged within a path of the liquid jet, the impingement member including an impingement surface shaped to redirect at least a portion of the liquid jet within the proximal chamber in the form of a second liquid jet.

In some embodiments, the liquid supply port can be disposed to direct the liquid jet to impinge on the impingement surface at a contact point superior to a vertical center of the impingement surface. The liquid supply port can be disposed to direct the liquid jet to impinge on the impingement surface at a contact point lateral to a horizontal center of the impingement member. The impingement surface can be shaped to redirect at least a portion of the liquid jet within the proximal chamber from a position lateral to the horizontal center of the impingement surface on a side of the impingement surface opposite the contact point. An angle between a vertical axis of the impingement surface and a radial line extending from a center point of the impingement surface through the contact point can be between −45° and 45°. The angle can be between −30° and 30°. The angle can be between −15° and 15°. The liquid jet can be disposed to impinge on the impingement surface at a contact point at a radial distance less than 0.63 inches from a center point of the impingement surface. The liquid jet can be disposed to impinge on the impingement surface at the contact point at a radial distance between 0.010 inches and 0.05 inches from the center point of the impingement surface. The liquid jet can be disposed to impinge on the impingement surface at a contact point at a radial distance between 1% and 49% of a diameter of the impingement surface. The liquid jet can be disposed to impinge on the impingement surface at the contact point at a radial distance between 5% and 45% of a diameter of the impingement surface. The liquid jet can be disposed to impinge on the impingement surface at the contact point at a radial distance between 8% and 40% of a diameter of the impingement surface. The liquid jet can be disposed to impinge on the impingement surface at the contact point at a radial distance between 15% and 25% of a diameter of the impingement surface. The liquid jet can be disposed to impinge on the impingement surface at the contact point at a radial distance between 20% and 40% of a diameter of the impingement surface. The impingement member can be angled downwardly towards the transition opening. A central axis of the impingement member can be angled inferiorly from an anterior-posterior axis of the proximal chamber by an angle between 0° and 10°. The central axis of the impingement member can be angled inferiorly from the anterior-posterior axis of the proximal chamber by an angle between 0° and 6°. The central axis of the impingement member can be angled inferiorly from the anterior-posterior axis of the proximal chamber by an angle between 0° and 3°. A central axis of the impingement member can be angled laterally relative to a superior-inferior axis of the proximal chamber. The liquid supply port can be disposed to direct the liquid jet along a jet axis angled superiorly to an anterior-posterior axis of the proximal chamber. The liquid supply port can be disposed to direct the liquid jet along the jet axis superiorly to the anterior-posterior axis of the proximal chamber by an angle between 0° and 10°. The liquid supply port can be disposed to direct the liquid jet along the jet axis superiorly to the anterior-posterior axis of the proximal chamber by an angle between 0° and 6°. The liquid supply port can be disposed to direct the liquid jet along the jet axis superiorly to the anterior-posterior axis of the proximal chamber by an angle between 0° and 4°. The liquid supply port can be disposed to direct the liquid jet along a jet axis angled laterally relative to a superior-inferior axis of the proximal chamber. The liquid jet can be disposed to impinge on the impingement surface at a contact point wherein the impingement surface can be angled to redirect at least a portion of the liquid jet within the proximal chamber in the form of a second liquid jet. The liquid jet can be disposed to impinge on the impingement surface at an angle relative to the impingement surface configured to cause the liquid jet to be redirected from the impingement surface in the form of a second liquid jet. The impingement surface can be hemispherical. The impingement surface can be concave. The liquid supply port and the impingement member can be arranged relative to each other to create a turbulent flow of liquid within the treatment region over a course of a treatment procedure. The apparatus can include a suction port exposed to the proximal chamber. The suction port can be disposed along an upper wall of the proximal chamber. The apparatus can include an outlet line connected to the suction port. The apparatus can include a vent exposed to ambient air, the vent in fluid communication with the outlet line and being positioned along the outlet line at a location downstream of the suction port. A treatment fluid within the proximal chamber and the distal chamber can include a substantially degassed treatment fluid. The liquid supply port can be disposed to direct the liquid stream to generate pressure waves in a treatment fluid within the proximal chamber and the distal chamber, the generated pressure waves having a broadband power spectrum. The liquid supply port can be disposed to direct the liquid jet to impinge on the impingement surface at a contact point inferior to a vertical center of the impingement surface. The impingement surface can be shaped to redirect at least a portion of the liquid jet in the form of a second liquid jet within the proximal chamber from a position superior to the vertical center of the impingement surface.

In another embodiment, a method for operating a dental instrument is provided. The method can include providing an access opening of the dental instrument configured to be placed in fluid communication with a treatment region of the tooth, directing a liquid stream over a transition opening between a proximal chamber and a distal chamber of the dental instrument to impinge on an impingement member of the dental instrument, and redirecting the liquid stream using one or more surfaces of the impingement member that is positioned to redirect at least a portion of the liquid stream across at least a portion of the transition opening.

In another embodiment, a method for operating a dental instrument is provided. The method can include providing an access opening of the dental instrument configured to be placed in fluid communication with a treatment region of the tooth, and directing a liquid stream over a transition opening between a proximal chamber and a distal chamber of the dental instrument to impinge on an impingement member of the dental instrument so as to create a turbulent flow of liquid within the proximal chamber.

In another embodiment, a method for operating a dental instrument is provided. The method can include providing an access opening of the dental instrument configured to be placed in fluid communication with a treatment region of the tooth, directing a liquid stream over a transition opening between a proximal chamber and a distal chamber of the dental instrument to impinge on an impingement member of the dental instrument, and redirecting the liquid stream using one or more surfaces of the impingement member that is positioned to redirect at least a portion of the liquid stream across at least a portion of the transition opening.

In another embodiment, a method for operating a dental instrument is provided. The method can include providing an access opening of the dental instrument configured to be placed in fluid communication with a treatment region of the tooth, directing a liquid stream over a transition opening between a proximal chamber and a distal chamber of the dental instrument to impinge on an impingement member of the dental instrument, and redirecting the liquid stream using one or more surfaces of the impingement member that is positioned to redirect at least a portion of the liquid stream across at least a portion of the transition opening.

In another embodiment, a method for operating a dental instrument is provided. The method can include providing an access opening of the dental instrument configured to be placed in fluid communication with a treatment region of the tooth, and directing a liquid stream over a transition opening between a proximal chamber and a distal chamber of the dental instrument, the proximal chamber including a first interior surface geometry, and the distal chamber including a second interior surface geometry different than the first interior surface geometry.

In another embodiment, a method for operating a dental instrument is provided. The method can include providing an access opening of the dental instrument configured to be placed in fluid communication with a treatment region of the tooth, the dental treatment apparatus including a proximal chamber, a distal chamber, and a non-uniform transition region between the proximal chamber and the distal chamber, and directing a liquid stream across the proximal chamber.

In some embodiments, of the above methods, the dental treatment instrument can include one or more flow disruptors positioned within the proximal chamber. The proximal chamber can have a first interior surface geometry and the distal chamber can have a second interior surface geometry different than the first interior surface geometry. The proximal chamber can include a non-uniform transition between the proximal chamber and the distal chamber. The dental instrument further includes a suction port exposed to the proximal chamber. The suction port can be disposed along an upper wall of the proximal chamber. The dental instrument can include an outlet line connected to the suction port. The dental instrument can include a vent exposed to ambient air, the vent in fluid communication with the outlet line and being positioned along the outlet line at a location downstream of the suction port. Directing the liquid stream can include directing the liquid stream to generate pressure waves in a treatment fluid within the proximal chamber and the distal chamber, the generated pressure waves having a broadband power spectrum. Directing the liquid stream over the transition opening between the proximal chamber and the distal chamber of the dental instrument to impinge on the impingement member of the dental instrument can include directing the liquid stream to impinge on the impingement member at a contact point superior to a vertical center of the impingement member. Redirecting the liquid stream using one or more surfaces of the impingement member can include redirecting the liquid stream using one or more surfaces shaped to redirect at least a portion of the liquid stream within the proximal chamber from a position inferior to the vertical center of the impingement member. Directing the liquid stream over the transition opening between the proximal chamber and the distal chamber of the dental instrument to impinge on the impingement member of the dental instrument can include directing the liquid stream to impinge on the impingement member at a contact point lateral to a horizontal center of the impingement member. Redirecting the liquid stream using one or more surfaces of the impingement member can include redirecting the liquid stream using one or more surfaces shaped to redirect at least a portion of the liquid stream within the proximal chamber from a position lateral to the horizontal center of the impingement member on a side of the impingement member opposite the contact point. Directing the liquid stream over the transition opening between the proximal chamber and the distal chamber of the dental instrument to impinge on the impingement member of the dental instrument can include directing the liquid stream to impinge on the impingement member at a contact point inferior to a vertical center of the impingement member. Redirecting the liquid stream using one or more surfaces of the impingement member can include redirecting the liquid stream using one or more surfaces shaped to redirect at least a portion of the liquid stream within the proximal chamber from a position superior to the vertical center of the impingement member. Directing the liquid stream can include directing a liquid jet, wherein redirecting the liquid jet using one or more surfaces of the impingement member can include redirecting the liquid jet using one or more surfaces of the impingement member configured to redirect at least a portion of the liquid jet in the form of a second liquid jet. Directing the liquid stream over the transition opening between the proximal chamber and the distal chamber of the dental instrument to impinge on the impingement member can include directing the liquid stream to impinge on the impingement member at a contact point superior to a vertical center of the impingement member. The method can further include redirecting the liquid stream using one or more surfaces of the impingement member shaped to redirect at least a portion of the liquid stream within the proximal chamber from a position inferior to the vertical center of the impingement member. Directing the liquid stream over the transition opening between the proximal chamber and the distal chamber of the dental instrument to impinge on the impingement member can include directing the liquid stream to impinge on the impingement member at a contact point lateral to a horizontal center of the impingement member. The method can further include redirecting the liquid stream using one or more surfaces of the impingement member shaped to redirect at least a portion of the liquid stream within the proximal chamber from a position lateral to the horizontal center of the impingement member on a side of the impingement member opposite the contact point. Directing the liquid stream over the transition opening between the proximal chamber and the distal chamber of the dental instrument to impinge on the impingement member can include directing the liquid stream to impinge on the impingement member at a contact point inferior to a vertical center of the impingement member. The method can further include redirecting the liquid stream using one or more surfaces of the impingement member shaped to redirect at least a portion of the liquid stream within the proximal chamber from a position superior to the vertical center of the impingement member. Directing the liquid stream can include directing a liquid jet, the method further including redirecting the liquid jet using one or more surfaces of the impingement member configured to redirect at least a portion of the liquid jet in the form of a second liquid jet. Directing the liquid stream can include directing the liquid stream to impinge on an impingement member of the dental instrument. Directing the liquid stream to impinge on the impingement member can include directing the liquid stream to impinge on the impingement member at a contact point superior to a vertical center of the impingement member. The method can further include redirecting the liquid stream using one or more surfaces of the impingement member shaped to redirect at least a portion of the liquid stream within the proximal chamber from a position inferior to the vertical center of the impingement member. Directing the liquid stream to impinge on the impingement member can include directing the liquid stream to impinge on the impingement member at a contact point lateral to a horizontal center of the impingement member. The method can further include redirecting the liquid stream using one or more surfaces of the impingement member shaped to redirect at least a portion of the liquid stream within the proximal chamber from a position lateral to the horizontal center of the impingement member on a side of the impingement member opposite the contact point. Directing the liquid stream to impinge on the impingement member can include directing the liquid stream to impinge on the impingement member at a contact point inferior to a vertical center of the impingement member. The method can further include redirecting the liquid stream using one or more surfaces of the impingement member shaped to redirect at least a portion of the liquid stream within the proximal chamber from a position superior to the vertical center of the impingement member. Directing the liquid stream to impinge on the impingement member can include directing a liquid jet to impinge on the impingement member, the method further including redirecting the liquid jet using one or more surfaces of the impingement member configured to redirect at least a portion of the liquid jet in the form of a second liquid jet.

In another embodiment, a method for operating a dental instrument is provided. The method can include providing an access opening of the dental instrument configured to be placed in fluid communication with a treatment region of the tooth, directing a liquid jet to impinge on an impingement surface of an impingement member within a chamber of the dental instrument at a contact point superior to a vertical center of the impingement surface, and redirecting at least a portion of the liquid jet within the chamber from a position inferior to the vertical center of the impingement surface using the impingement surface.

In some embodiments, directing the liquid jet to impinge on the impingement surface can include directing the liquid jet to impinge on the impingement surface at the contact point lateral to a horizontal center of the impingement surface. Redirecting the liquid jet can include redirecting at least a portion of the liquid jet within the chamber from a position lateral to the horizontal center of the impingement surface on a side of the impingement surface opposite the contact point. An angle between a vertical axis of the impingement surface and a radial line extending from a center point of the impingement surface through the contact point can be between −45° and 45°. The angle can be between −30° and 30°. The angle can be between −15° and 15°. Directing the liquid jet to impinge on the impingement surface can include directing the liquid jet to impinge on the impingement surface at the contact point at a radial distance less than 0.63 inches from a center point of the impingement surface. Directing the liquid jet to impinge on the impingement surface can include directing the liquid jet to impinge on the impingement surface at the contact point at a radial distance between 0.010 inches and 0.05 inches from the center point of the impingement surface. Directing the liquid jet to impinge on the impingement surface can include directing the liquid jet to impinge on the impingement surface at the contact point at a radial distance between 1% and 49% of a diameter of the impingement surface. Directing the liquid jet to impinge on the impingement surface can include directing the liquid jet to impinge on the impingement surface at the contact point at a radial distance between 5% and 45% of a diameter of the impingement surface. Directing the liquid jet to impinge on the impingement surface can include directing the liquid jet to impinge on the impingement surface at the contact point at a radial distance between 8% and 40% of a diameter of the impingement surface. Directing the liquid jet to impinge on the impingement surface can include directing the liquid jet to impinge on the impingement surface at the contact point at a radial distance between 15% and 25% of a diameter of the impingement surface. Directing the liquid jet to impinge on the impingement surface can include directing the liquid jet to impinge on the impingement surface at the contact point at a radial distance between 20% and 40% of a diameter of the impingement surface. The chamber can include a proximal chamber, wherein the impingement member can be angled downwardly towards a transition opening between the proximal chamber and a distal chamber of the dental apparatus. A central axis of the impingement member can be angled inferiorly from an anterior-posterior axis of the chamber by an angle between 0° and 10°. The central axis of the impingement member can be angled inferiorly from the anterior-posterior axis of the chamber by an angle between 0° and 6°. The central axis of the impingement member can be angled inferiorly from the anterior-posterior axis of the chamber by an angle between 0° and 3°. A central axis of the impingement member can be angled laterally relative to a superior-inferior axis of the chamber. Directing the liquid jet to impinge on the impingement surface can include directing the liquid jet along a jet axis angled superiorly to an anterior-posterior axis of the chamber. Directing the liquid jet to impinge on the impingement surface can include directing the liquid jet along the jet axis superiorly to the anterior-posterior axis of the chamber by an angle between 0° and 10°. Directing the liquid jet to impinge on the impingement surface can include directing the liquid jet along the jet axis superiorly to the anterior-posterior axis of the chamber by an angle between 0° and 6°. Directing the liquid jet to impinge on the impingement surface can include directing the liquid jet along the jet axis superiorly to the anterior-posterior axis of the chamber by an angle between 0° and 4°. Directing the liquid jet to impinge on the impingement surface can include directing the liquid jet along a jet axis angled laterally relative to a superior-inferior axis of the chamber. The impingement surface can be shaped to redirect at least a portion of the liquid jet within the chamber in the form of a second liquid jet. The impingement surface can be angled at the contact point to redirect at least a portion of the liquid jet within the chamber in the form of a second liquid jet. Directing the liquid jet to impinge on the impingement surface can include directing the liquid jet impinge on the impingement surface at an angle relative to the impingement surface configured to cause the liquid jet to be redirected from the impingement surface in the form of a second liquid jet. The impingement surface can be hemispherical. The impingement surface can be concave. A liquid supply port of the dental instrument and the impingement member can be arranged relative to each other to create a turbulent flow of liquid within the chamber. The dental instrument can include a suction port exposed to the chamber. The suction port can be disposed along an upper wall of the chamber. The dental instrument can include an outlet line connected to the suction port. The dental instrument can include a vent exposed to ambient air, the vent in fluid communication with the outlet line and being positioned along the outlet line at a location downstream of the suction port. A fluid within the chamber can include a substantially degassed fluid. Directing the liquid jet to impinge on the impingement surface can include generating pressure waves in a fluid within the chamber, the generated pressure waves having a broadband power spectrum.

In another embodiment, a method for operating a dental instrument is provided. The method can include providing an access opening of the dental instrument configured to be placed in fluid communication with a treatment region of the tooth, and directing a liquid jet to impinge on an impingement surface of an impingement member within a chamber of the dental instrument so as to redirect at least a portion of the liquid jet from the impingement member in the form of a second liquid jet.

In some embodiments, directing the liquid jet to impinge on the impingement surface can include directing the liquid jet to impinge on the impingement surface at a contact point superior to a vertical center of the impingement surface. Directing the liquid jet to impinge on the impingement surface can include directing the liquid jet to impinge on the impingement surface at the contact point lateral to a horizontal center of the impingement surface. The impingement surface can be shaped to redirect at least a portion of the liquid jet within the chamber from a position lateral to the horizontal center of the impingement surface on a side of the impingement surface opposite the contact point. An angle between a vertical axis of the impingement surface and a radial line extending from a center point of the impingement surface through the contact point can be between −45° and 45°. The angle can be between −30° and 30°. The angle can be between −15° and 15°. Directing the liquid jet to impinge on the impingement surface can include directing the liquid jet to impinge on the impingement surface at the contact point at a radial distance less than 0.63 inches from a center point of the impingement surface. Directing the liquid jet to impinge on the impingement surface can include directing the liquid jet to impinge on the impingement surface at the contact point at a radial distance between 0.010 inches and 0.05 inches from the center point of the impingement surface. Directing the liquid jet to impinge on the impingement surface can include directing the liquid jet to impinge on the impingement surface at the contact point at a radial distance between 1% and 49% of a diameter of the impingement surface. Directing the liquid jet to impinge on the impingement surface can include directing the liquid jet to impinge on the impingement surface at the contact point at a radial distance between 5% and 45% of a diameter of the impingement surface. Directing the liquid jet to impinge on the impingement surface can include directing the liquid jet to impinge on the impingement surface at the contact point at a radial distance between 8% and 40% of a diameter of the impingement surface. Directing the liquid jet to impinge on the impingement surface can include directing the liquid jet to impinge on the impingement surface at the contact point at a radial distance between 15% and 25% of a diameter of the impingement surface. Directing the liquid jet to impinge on the impingement surface can include directing the liquid jet to impinge on the impingement surface at the contact point at a radial distance between 20% and 40% of a diameter of the impingement surface. The chamber can include a proximal chamber, wherein the impingement member can be angled downwardly towards a transition opening between the proximal chamber and a distal chamber of the instrument. A central axis of the impingement member can be angled inferiorly from an anterior-posterior axis of the chamber by an angle between 0° and 10°. The central axis of the impingement member can be angled inferiorly from the anterior-posterior axis of the chamber by an angle between 0° and 6°. The central axis of the impingement member can be angled inferiorly from the anterior-posterior axis of the chamber by an angle between 0° and 3°. A central axis of the impingement member can be angled laterally relative to a superior-inferior axis of the chamber. Directing the liquid jet to impinge on the impingement surface can include directing the liquid jet along a jet axis angled superiorly to an anterior-posterior axis of the chamber. Directing the liquid jet to impinge on the impingement surface can include directing the liquid jet along the jet axis superiorly to the anterior-posterior axis of the chamber by an angle between 0° and 10°. Directing the liquid jet to impinge on the impingement surface can include directing the liquid jet along the jet axis superiorly to the anterior-posterior axis of the chamber by an angle between 0° and 6°. Directing the liquid jet to impinge on the impingement surface can include directing the liquid jet along the jet axis superiorly to the anterior-posterior axis of the chamber by an angle between 0° and 4°. Directing the liquid jet to impinge on the impingement surface can include directing the liquid jet along a jet axis angled laterally relative to a superior-inferior axis of the chamber. The impingement surface can be shaped to redirect at least a portion of the liquid jet within the chamber in the form of the second liquid jet. The impingement surface can be angled at the contact point to redirect at least a portion of the liquid jet within the chamber in the form of the second liquid jet. Directing the liquid jet to impinge on the impingement surface can include directing the liquid jet impinge on the impingement surface at an angle relative to the impingement surface configured to cause the liquid jet to be redirected from the impingement surface in the form of the second liquid jet. The impingement surface can be hemispherical. The impingement surface can be concave. A liquid supply port of the dental instrument and the impingement member can be arranged relative to each other to create a turbulent flow of liquid within the chamber. The dental instrument can include a suction port exposed to the chamber. The suction port can be disposed along an upper wall of the chamber. The dental instrument can include an outlet line connected to the suction port. The dental instrument can include a vent exposed to ambient air, the vent in fluid communication with the outlet line and being positioned along the outlet line at a location downstream of the suction port. A fluid within the chamber can include a substantially degassed fluid. Directing the liquid jet to impinge on the impingement surface can include generating pressure waves in a fluid within the chamber, the generated pressure waves having a broadband power spectrum.

In another embodiment, an apparatus for applying a platform to a tooth is provided. The apparatus can include one or more surfaces configured to receive a conforming material, a handle extending proximally from the one or more surfaces, a pin extending distally from the one or more surfaces and configured to be received within an access opening of the tooth; and a venting pathway extending through the pin and handle.

In some embodiments, the apparatus can include an upper rim including an upper surface, lower surface, and an outer edge extending therebetween, and a lower rim extending inferiorly from the upper rim and including a lower surface and an outer edge extending between the lower surface and the upper rim, wherein the one or more surfaces configured to receive the conforming material include the lower surface of the upper rim, the outer edge of the lower rim, and the lower surface of the lower rim. The upper rim can have a larger cross-section than the lower rim. The upper rim and the lower rim can be each shaped in the form of a disc. The upper rim can have a circular cross-section and the lower rim can have a circular cross-section. The outer edge of the upper rim can extend radially beyond the outer edge of the lower rim. The pin can be tapered between a proximal end of the pin and a distal end of the pin. The venting pathway can extend from a proximal-most end of the handle to a distal-most end of the pin. The handle can include an elongated handle top. The handle can include one or more circumferential ridges. The venting pathway can include a first venting pathway, wherein the apparatus includes a second venting pathway. The first venting pathway can extend along a first axis and the second venting pathway can extend along a second axis transverse to the first axis. The second axis can be perpendicular to the first axis. The second venting pathway can include a recess extending inferiorly from a superior-most surface of the handle and at least partially laterally relative to the first venting pathway. The second venting pathway can include a channel extending laterally through a portion of the handle and at least partially laterally relative to the first venting pathway. The channel can include a through-hole. The second venting pathway can be in fluid communication with the first venting pathway. The one or more surfaces can be shaped to form a platform from the conforming material including a bottom surface, an access opening extending through the bottom surface, and a ridge extending superiorly from the bottom surface. The bottom surface can be configured to receive a dental treatment instrument. The ridge can be configured to restrict lateral movement of the dental treatment instrument across the bottom surface of the platform.

In another embodiment, a method for treating a tooth is provided. The method can include applying a conforming material to one or more surfaces of an applicator around a pin extending distally beyond the surface of the applicator, advancing the applicator towards the tooth to position the pin of the applicator within an access opening of the tooth and apply the conforming material to a top surface of the tooth, and curing the conforming material while the conforming material is positioned on the top surface of the tooth to form a platform on the top surface of the tooth.

In some embodiments, the conforming material can include a light cure resin. Curing the conforming material while the conforming material is positioned on the top surface of the tooth to form the platform on the top surface of the tooth can include forming a platform including a bottom surface, an access opening extending through the bottom surface, and a ridge extending superiorly from the bottom surface. The access opening of the platform can align with the access opening of the tooth. The method can include positioning a dental treatment instrument on the platform so that the dental treatment instrument can be in fluid communication with the access opening of the tooth via the access opening of the platform. The ridge of the platform can be configured to restrict lateral movement of the dental treatment instrument across the bottom surface of the platform. The method can include removing the applicator from the platform and reforming the size or shape of the access opening of the platform. Reforming the size and shape of the access opening of the platform can include reforming the size and shape of the access opening of the platform to conform to the access opening of the tooth. The applicator can include the one or more surfaces of the applicator, wherein the one or more surfaces can be configured to receive the conforming material, a handle extending proximally from the one or more surfaces, the pin, wherein the pin extends distally from the one or more surfaces, and a venting pathway extending through the pin and handle. The applicator can further include an upper rim including an upper surface, lower surface, and an outer edge extending therebetween, and a lower rim extending inferiorly from the upper rim and including a lower surface and an outer edge extending between the lower surface and the upper rim, wherein the one or more surfaces configured to receive the conforming material include the lower surface of the upper rim, the outer edge of the lower rim, and the lower surface of the lower rim. The upper rim can have a larger cross-section than the lower rim. The upper rim and the lower rim can be each shaped in the form of a disc. The upper rim can have a circular cross-section and the lower rim can have a circular cross-section. The outer edge of the upper rim can extend radially beyond the outer edge of the lower rim. The venting pathway can extend from a proximal-most end of the handle to a distal-most end of the pin. The handle can include an elongated handle top. The handle can include one or more circumferential ridges. The venting pathway can include a first venting pathway, wherein the applicator includes a second venting pathway. The first venting pathway can extend along a first axis and the second venting pathway can extend along a second axis transverse to the first axis. The second axis can be perpendicular to the first axis. The second venting pathway can include a recess extending inferiorly from a superior-most surface of the handle and at least partially laterally relative to the first venting pathway. The second venting pathway can include a channel extending laterally through a portion of the handle and at least partially laterally relative to the first venting pathway. The channel can include a through-hole. The second venting pathway can be in fluid communication with the first venting pathway. The pin can be tapered between a proximal end of the pin and a distal end of the pin.

In another embodiment, an apparatus for treating a tooth is provided. The apparatus can include a chamber having an access opening to provide fluid communication with a treatment region of the tooth, a liquid supply port disposed to direct a liquid jet into the chamber to create pressure waves within the chamber, and at least one oscillatory member exposed to fluid motion in the chamber, the fluid motion causing the at least one oscillatory member to oscillate.

In some embodiments, the at least one oscillatory member is configured oscillate to amplify an amplitude of at least one frequency of the pressure waves within the chamber. The liquid supply port can be disposed to direct the liquid jet into the chamber to create fluid motion in the chamber, wherein the at least one oscillatory member can be configured to oscillate in response to the fluid motion. The apparatus can include an impingement member arranged within a path of the liquid jet, the impingement member having one or more surfaces positioned to redirect at least a portion of the liquid jet within the chamber. The at least one oscillatory member can be configured to oscillate at a natural frequency that corresponds to the at least one frequency of the pressure waves. The at least one oscillatory member can include a plurality of oscillatory members. Each of the plurality of oscillatory members can be configured to oscillate to amplify the amplitude of a different frequency of the pressure waves. Each of the plurality of oscillatory members can have a different shape. Each of the plurality of oscillatory members can have a different size. Each of the plurality of oscillatory members can be positioned at a different location. Each of the plurality of oscillatory members can be configured to oscillate at a different natural frequency. The pressure waves can include a range of frequencies effective for cleaning a treatment region of the tooth, wherein the at least one oscillatory member can be configured to oscillate to amplify the amplitude of at least one frequency in the range of frequencies. The at least one oscillatory member can be configured to oscillate at a natural frequency that corresponds to at least one frequency in the range of frequencies. The at least one oscillatory member can include a plurality of oscillatory members. Each of the plurality of oscillatory members can be configured to oscillate to amplify the amplitude a different frequency within the range of frequencies. Each of the plurality of oscillatory members can be configured to oscillate at a different natural frequency corresponding to a frequency within the range of frequencies.

In another embodiment, an apparatus for treating a tooth is provided. The apparatus can include a chamber having an access opening to provide fluid communication with a treatment region of the tooth, a liquid supply port disposed to direct a liquid jet into the chamber to create pressure waves within the chamber, and at least one movable member exposed to fluid motion in the chamber, the fluid motion causing the at least one movable member to move.

In some embodiments, the liquid supply port can be disposed to direct the liquid jet into the chamber to create fluid motion in the chamber, wherein the at least one movable member can be configured to move in response to the fluid motion. The apparatus can include an impingement member arranged within a path of the liquid jet, the impingement member having one or more surfaces positioned to redirect at least a portion of the liquid jet within the chamber. The at least one movable member can include a plurality of movable members. Each of the plurality of movable members can have a different shape. Each of the plurality of movable members can have a different size. Each of the plurality of movable members can be positioned at a different location.

In another embodiment, a method of treating a carious on an exterior surface of a tooth is provided. The method can include treating the carious region on the exterior surface of the tooth using a treatment instrument comprising a pressure wave generator. Treating the carious region on the tooth can include propagating pressure waves through a treatment liquid with the pressure wave generator to reduce infection of the carious region. Treating the carious region on the tooth can further include, after the propagating, neutralizing an acidity of at least the carious region. Treating the carious region on the tooth can also include remineralizing at least the carious region.

In some embodiments, the method of treating a carious above can further include sealing and/or remineralizing the tooth. In some embodiments, the method of treating a carious above can further include imaging the tooth to evaluate the existence and/or condition of carious tissue in the carious region. In some embodiments, imaging the tooth comprising using an intra oral camera to detect caries utilizing autofluorescence. In some embodiments, the method of treating a carious above can further include imaging the tooth before treating the caries on the tooth. In some embodiments, the method of treating a carious above can further include imaging the tooth after treating the caries on the tooth. In some embodiments, the method of treating a carious above can further include imaging the tooth after sealing and/or remineralizing the tooth. In some embodiments, neutralizing an acidity of at least the carious region comprises delivering an alkaline fluid to the carious region. In some embodiments, remineralizing at least the carious region comprises delivering a fluid containing fluoride and/or calcium ions to the carious region. In some embodiments, treating the carious region comprises positioning a fluid platform against the tooth over the carious region. In some embodiments, the method above further includes providing a fluid seal between the fluid platform and the tooth.

In another embodiment, a method of treating a carious region on an exterior surface of a tooth is provided. The method can include imaging the tooth to detect the caries. The method can include, after imaging, positioning a treatment instrument on the exterior surface of the tooth over the carious region. The method can further include activating a pressure wave generator of the treatment instrument to propagate pressure waves through a treatment fluid to at least partially disinfect the carious region. The method can also include, after the activating, performing one or more of neutralizing or remineralizing the carious region of the tooth.

In some embodiments, the method of treating a carious region above can further include sealing and/or remineralizing the tooth. In some embodiments, imaging the tooth comprising using an intra oral camera to detect caries utilizing autofluorescence. In some embodiments, the method above can further include imaging the tooth after treating the caries on the tooth. In some embodiments, the method above can further include imaging the tooth after sealing and/or remineralizing the tooth. In some embodiments, neutralizing the carious region of the tooth comprises delivering an alkaline fluid to the carious region. In some embodiments, remineralizing the carious region of the tooth comprises delivering a fluid containing fluoride and/or calcium ions to the carious region. In some embodiments, positioning the treatment instrument comprises positioning a fluid platform against the tooth such that a chamber of the fluid platform is disposed over the carious region. In some embodiments, the method above further includes providing a fluid seal between the fluid platform and the tooth.

In some embodiments, a method of treating a carious region on an exterior surface of a tooth is provided. The method can include treating the carious region on the exterior surface of the tooth using a treatment instrument including a pressure wave generator. The method can further include treating the carious region on the tooth with the treatment instrument by propagating pressure waves through a treatment liquid with the pressure wave generator to reduce infection of the carious region. The method can include neutralizing an acidity of at least the carious region with a buffering solution. Additionally, the method can include remineralizing at least the carious region.

In some embodiments, the method of treating a carious region can further include sealing and/or remineralizing the tooth. In some embodiments, the method can include imaging the tooth to evaluate an existence and/or condition of carious tissue in the carious region. In some embodiments, the method can include imaging the tooth by using an intra oral camera to detect caries utilizing autofluorescence. The method can include imaging the tooth before treating the carious region on the tooth. The method can also include imaging the tooth after treating the carious region on the tooth. Additionally, the method can include imaging the tooth after sealing and/or remineralizing the tooth.

In some embodiments, the method can include disinfecting bacteria of at least the carious region by delivering an alkaline fluid to the carious region. The method can include remineralizing at least the carious region includes delivering a fluid containing fluoride and/or calcium ions to the carious region. The method can include treating the carious region by positioning a fluid platform against the tooth over the carious region. In some embodiments, the method can include providing a fluid seal between the fluid platform and the tooth. Furthermore, in some embodiments the buffering solution includes a phosphate buffered saline (PBS). In some embodiments, the method can include treating the carious region includes delivering a disinfecting solution to destroy bacteria, where treating the carious region includes delivering the buffering solution after delivering the disinfecting solution to neutralize an acidity of the carious region, where the treatment liquid includes the disinfecting solution. In some embodiments, the method can include treating the carious region by propagating pressure waves through a sodium hypochlorite treatment liquid to disinfect the carious region and, subsequently, propagating pressure waves through a buffering solution to neutralize the carious region, where treating the carious region includes significantly reducing a presence of free calcium ions at the carious region. The method can include a buffering solution configured to neutralize the acidity present at an incipient lesion.

In some embodiments, a method of treating a carious region on an exterior surface of a tooth is provided. The method can include imaging the tooth to detect caries. The method can further include, after imaging, positioning a treatment instrument on the exterior surface of the tooth over the carious region. Additionally, the method can include activating a pressure wave generator of the treatment instrument to propagate pressure waves through a treatment fluid to at least partially disinfect the carious region. The method can also include one or more of neutralizing the carious region of the tooth with a buffering solution or remineralizing the carious region of the tooth.

In some embodiments, the method can include sealing and/or remineralizing the tooth. The method can include imaging the tooth including using an intra oral camera to detect the caries utilizing autofluorescence. The method can also include imaging the tooth after treating the caries on the tooth. In some embodiments, the method can include imaging the tooth after sealing and/or remineralizing the tooth. In some embodiments, the method can include disinfecting the carious region of the tooth includes delivering an alkaline fluid to the carious region. The method can include remineralizing the carious region of the tooth includes delivering a fluid containing fluoride and/or calcium ions to the carious region. In some embodiments, the method can include positioning the treatment instrument includes positioning a fluid platform against the tooth such that a chamber of the fluid platform is disposed over the carious region. In some embodiments, the method can include providing a fluid seal between the fluid platform and the tooth. The method can include a buffering solution having a phosphate buffered saline (PBS).

In some embodiments disclosed herein, a system for treating a tooth having a carious region is provided. The system can include a fluid platform having a chamber sized and shaped to retain fluid. The chamber can be configured to be coupled to and at least partially seal against an external surface of the tooth over the carious region. A pressure wave generator can be exposed to the chamber and configured to generate pressure waves in the retained fluid sufficient to treat the carious region. The system can further include a controller configured to cause a delivery of a disinfecting solution to the carious region and to cause a delivery of a buffering solution to the carious region.

In some embodiments, the system can include a controller which can be configured to cause a delivery of a disinfecting solution to the carious region in a first step and can be configured to cause a delivery of a buffering solution in a subsequent second step, wherein the first step follows the second step. The system can include a pressure wave generator which can be configured to generate pressure waves within the disinfecting solution to disinfect the carious region when the chamber is substantially filled with fluid. The system can include a disinfecting solution that can include sodium hypochlorite. In some embodiments, the system can include a pressure wave generator which can be configured to generate pressure waves with the buffering solution to neutralize the carious region. In some embodiments, the system can include a buffering solution which can include a phosphate buffered saline (PBS). Additionally, in some embodiments, the controller can be configured to cause the pressure wave generator to propagate pressure waves with the disinfecting solution for a desired period of time to disinfect the carious region, wherein the controller is configured to cause the pressure wave generator to propagate pressure waves with the buffering solution to neutralize the carious region. Further, in some embodiments, the controller can be configured to activate the pressure wave generator to propagate pressure waves with the disinfecting solution before propagating pressure waves with the buffering solution. The system can include a pressure wave generator which can be configured to remineralize the carious region of the tooth.

In some embodiments, the system can include a matrix having a proximal surface coupled to the fluid platform and a distal surface coupled to the tooth, where the matrix is configured to facilitate cleaning and filling of the carious region of the tooth. The system can include a matrix which can have a channel extending from a superior end of the matrix to an inferior end of the matrix. The channel, in some embodiments of the matrix, can be coupled to a wall of a conforming material, wherein an access opening of the conforming material corresponds to the channel of the matrix, wherein the access opening is in fluid communication with an opening of the tooth.

In some embodiments disclosed herein, a system for treating a tooth having a carious region is provided. The system can include a console including a user interface. The system can further include a control system operably coupled with the console and configured to cause a delivery of a disinfecting solution, a buffering solution, and a remineralizing fluid to treat the carious region. The control system of the system can be configured to activate a pressure wave generator to deliver pressure waves through the disinfecting solution to disinfect the carious region. The control system can be configured to activate the pressure wave generator to deliver pressure waves through the buffering solution to neutralize an acidity of the carious region. In some embodiments, the control system can be configured to cause delivery of the remineralizing fluid to the carious region.

In some embodiments, the system can include a fluid platform coupled to the console having a chamber sized and shaped to retain fluid, the chamber can be configured to be coupled to and at least partially seal against an external surface of the tooth over the carious region.

In some embodiments, the system can include the pressure wave generator which can be exposed to the chamber and configured to generate pressure waves in the retained fluid sufficient to treat the carious region. In some embodiments, the system can include the control system which can be configured to cause the pressure wave generator to propagate pressure waves with the disinfecting solution for a desired period of time to disinfect the carious region, the control system can be configured deliver a signal to the pressure wave generator to propagate pressure waves with the buffering solution in order to neutralize the carious region. The system can include a pressure wave generator which can be configured to generate pressure waves with the disinfecting solution to disinfect the carious region. The buffering solution can include a phosphate buffered saline (PBS). The disinfecting solution can include sodium hypochlorite. Further, in some embodiments, activating the pressure wave generator can include activating a valve positioned within a conduit.

In some embodiments disclosed herein, a system for treating a tooth having a carious region is provided. The system can include a fluid platform having a chamber sized and shaped to retain fluid. The system can further include a matrix including a disc-shaped body, the matrix having an upper surface superior to the disc-shaped body shaped to support the fluid platform and the chamber, the matrix having a lower surface inferior to the disc-shaped body configured to support and at least partially seal against an external surface of the tooth over the carious region, the matrix including a rim extending superiorly from the upper surface such that the upper surface is recessed below the rim. The system can also include a pressure wave generator exposed to the chamber and configured to generate pressure waves in the retained fluid sufficient to treat the carious region.

In some embodiments, the system can include the retained fluid which can include a disinfecting solution and a buffering solution. The system can further include a controller which can be configured to cause a delivery of the disinfecting solution to the carious region in a first step and which can cause the delivery of the buffering solution to the carious region in a second step. They system can include the matrix which can include a channel, where the channel can extend from the upper surface of the matrix to the lower surface of the matrix. In some embodiments, the system can include the rim which can have an upper edge, where the rim can include a lower edge positioned below the upper edge, where the rim can include an inner wall positioned between the upper edge and the lower edge, where the rim can be configured to engage a bottom cap of a fluid platform.

In some embodiments, the system can include a handle extends distally from an outer surface disc-shaped body of the matrix. In some embodiments, the handle can be configured to allow a user to position the matrix on the fluid platform. The matrix can have an access opening, where the access opening can include a first region and a second region. The second region can inferior to the first region. The first region can be configured to couple to the fluid platform to form a passageway from the fluid platform to the tooth.

In some embodiments, the pressure wave generator can be configured to generate pressure waves with the disinfecting solution to disinfect the carious region when the chamber is substantially filled with fluid. Furthermore, the buffering solution can include a phosphate buffered saline (PBS). In some embodiments, the disinfecting solution can include sodium hypochlorite. The system can include the fluid platform and the pressure wave generator can be coupled together and contained within a treatment instrument. In some embodiments, the method can include a kit which can include a treatment instrument containing the pressure wave generator and the fluid platform.

In some embodiments disclosed herein, a method of treating a carious region on an exterior surface of a tooth is provided. The method can include positioning a treatment instrument including a matrix on the exterior surface of the tooth over the carious region. The method can further include activating a pressure wave generator of the treatment instrument to propagate pressure waves through a treatment fluid to at least partially disinfect the carious region. Additionally, the method can include performing one or more of neutralizing the carious region of the tooth with a buffering solution or remineralizing the carious region of the tooth.

In some embodiments, the method includes the matrix which can have a channel, where the channel can extend from an upper surface of the matrix to a lower surface of the matrix. The method can, in some embodiments, include the matrix which can include an upper surface and a lower surface, where the upper surface can be coupled to the treatment instrument and the lower surface can be coupled to the exterior surface of the tooth, where the matrix includes a rim.

In some embodiments, the method can include an outer surface of the matrix which can be generally disc shaped, where a handle distally extends from an outer surface of the matrix. In some embodiments, the method can include the handle which can be configured to allow a user to position the matrix on the treatment instrument. The method can also include a pressure wave generator can be configured to generate pressure waves with the treatment fluid to disinfect the carious region when a chamber formed by the matrix and the treatment instrument is substantially filled with fluid. The method can include wherein the buffering solution includes a phosphate buffered saline (PBS). The method can also include the treatment fluid which can include sodium hypochlorite. In some embodiments, the method can include imaging the tooth, wherein positioning the treatment instrument including a matrix occurs after imaging the tooth.

In some embodiments disclosed herein, a method of treating a carious region on an exterior surface of a tooth is provided. The method can include treating the carious region on the exterior surface of the tooth using a treatment instrument which can include a pressure wave generator. The method can further include treating the carious region on the tooth by delivering a disinfecting solution to destroy bacteria using pressure waves generated by the pressure wave generator, and after delivering the disinfecting solution, delivering a buffering solution to neutralize an acidity of at least the carious region using pressure waves generated by the pressure wave generator.

In some embodiments, the method can include the pressure wave generator which can be configured to generate pressure waves with the disinfecting solution to disinfect the carious region when a chamber of the treatment instrument is substantially filled with the disinfecting solution. The method can include the pressure wave generator which can be configured to generate pressure waves with the buffering solution to neutralize the carious region when a chamber of the treatment instrument is substantially filled with the buffering solution. In some embodiments, the method can include the buffering solution including a phosphate buffered saline (PBS). In some embodiments, the method can include the disinfecting solution including sodium hypochlorite.

In some embodiments disclosed herein, a dental support device is provided. The dental support device can include a matrix having a disc-shaped body. The matrix can include an upper surface which can be superior to the disc-shaped body shaped to support a fluid platform of a treatment instrument. The matrix can have a lower surface inferior to the disc-shaped body and which can be configured to support and at least partially seal against an external surface of a tooth. The matrix can include a rim extending superiorly from the upper surface such that the upper surface is recessed below the rim.

In some embodiments, the dental support device can include the matrix which can a channel, wherein the channel extends from the upper surface of the matrix to the lower surface of the matrix. The device can include a rim, where the rim can include an upper edge, where the rim can include a lower edge positioned below the upper edge, where the rim can include an inner wall positioned between the upper edge and the lower edge, where the rim can be configured to engage a bottom cap of a fluid platform. The device can include a handle which may extend distally from an outer surface disc-shaped body of the matrix. The device can include the handle which can be configured to allow a user to position the matrix on the fluid platform. The device can include the matrix which can have an access opening, where the access opening can include a first region and a second region, where the second region can be inferior to the first region, where the first region can be configured to couple to the fluid platform to form a passageway from the fluid platform to the tooth.

For purposes of this summary, certain aspects, advantages, and novel features of certain disclosed inventions are summarized. It is to be understood that not necessarily all such advantages may be achieved in accordance with any particular embodiment of the invention. Thus, for example, those skilled in the art will recognize that the inventions disclosed herein may be embodied or carried out in a manner that achieves one advantage or group of advantages as taught herein without necessarily achieving other advantages as may be taught or suggested herein. Further, the foregoing is intended to summarize certain disclosed inventions and is not intended to limit the scope of the inventions disclosed herein.

### BRIEF DESCRIPTION OF THE DRAWINGS

The foregoing and other features, aspects, and advantages of the embodiments of the apparatus and methods of treating teeth (e.g., cleaning teeth) are described in detail below with reference to the drawings of various embodiments, which are intended to illustrate and not to limit the embodiments of the invention. The drawings comprise the following figures in which:

FIG. 1A is a schematic diagram of a system that includes components capable of removing unhealthy or undesirable materials from a root canal of a tooth.

FIG. 1B is a schematic diagram of a system that includes components capable of removing unhealthy or undesirable material from a treatment region on an exterior surface of a tooth.

FIG. 2A is a schematic perspective view of a treatment instrument according to some embodiments.

FIG. 2B is a magnified schematic perspective view of a fluid platform disposed at a distal end portion of a handpiece of the treatment instrument of FIG. 2A.

FIG. 2C is a schematic bottom perspective view of the treatment instrument of FIG. 2A.

FIG. 2D is a schematic side sectional view of the treatment instrument of FIG. 2A, taken along section 2D-2D of FIG. 2A.

FIG. 2E is a magnified bottom perspective sectional view of the fluid platform.

FIG. 2F is a magnified view of the fluid platform shown in the section of FIG. 2D.

FIG. 2G is a schematic side sectional view of the fluid platform taken along section 2G-2G of FIG. 2A.

FIG. 2H is a top perspective sectional view of the fluid platform taken along section 2H-2H of FIG. 2F.

FIG. 2I is a top perspective sectional view of the fluid platform taken along section 2I-2I of FIG. 2F.

FIG. 2J is a top plan view of the fluid platform taken along section 2J-2J of FIG. 2F.

FIG. 2K is a top plan view of the fluid platform taken along section 2I-2I of FIG. 2F.

FIG. 3A is top perspective view of a fluid platform according to some embodiments.

FIG. 3B is a bottom perspective view of the fluid platform of FIG. 3A.

FIG. 3C is a perspective exploded view of the fluid platform of FIG. 3A.

FIG. 3D is a side cross-sectional view of the fluid platform of FIG. 3A.

FIG. 3E is a rear cross-sectional view of the fluid platform of FIG. 3A.

FIG. 3F is a top perspective sectional view of the fluid platform of FIG. 3A.

FIG. 3G is a side cross-sectional view of the fluid platform of FIG. 3A.

FIG. 3H is a top cross-sectional view of the fluid platform of FIG. 3A.

FIG. 4A is a top perspective view of a fluid platform according to some embodiments.

FIG. 4B is a bottom perspective view of the fluid platform of FIG. 4A.

FIG. 4C is a side cross-sectional view of the fluid platform of FIG. 4A.

FIG. 4D is a top perspective sectional view of the fluid platform of FIG. 4A.

FIG. 4E is a top cross-sectional view of the fluid platform of FIG. 4A.

FIG. 5A is a side cross-sectional view of a fluid platform according to some embodiments.

FIG. 5B is a top perspective view of an impingement ring of the fluid platform of FIG. 5A.

FIG. 5C is a bottom perspective sectional view of the fluid platform of FIG. 5A.

FIG. 5D is a top perspective sectional view of the fluid platform of FIG. 5A.

FIG. 5E is a top cross-sectional view of the fluid platform of FIG. 5A.

FIG. 6A is a side cross-sectional view showing dimensions according to the fluid platforms of FIGS. 4A and 5A.

FIG. 6B is a top cross-sectional view showing dimensions according to the fluid platforms of FIGS. 4A and 5A.

FIG. 7A is a perspective exploded view of a fluid platform according to some embodiments.

FIG. 7B is a top perspective view of an impingement ring of the fluid platform of FIG. 7A.

FIG. 7C is a side cross-sectional view of the fluid platform of FIG. 7A.

FIG. 7D is a bottom perspective sectional view of the fluid platform of FIG. 7A.

FIG. 7E is a top perspective sectional view of the fluid platform of FIG. 7A.

FIG. 7F is a top cross-sectional view of the fluid platform of FIG. 7A.

FIG. 8A is a perspective exploded view of a fluid platform according to some embodiments.

FIG. 8B is a top perspective sectional view of the fluid platform of FIG. 8A.

FIG. 8C is a top perspective sectional view of the fluid platform of FIG. 8A.

FIG. 8D is a side cross-sectional view of the fluid platform of FIG. 8A.

FIG. 8E is a side cross-sectional view of the fluid platform of FIG. 8A.

FIG. 8F is a top cross-sectional view of the fluid platform of FIG. 8A.

FIG. 9A is a side cross-sectional view of a fluid platform according to some embodiments.

FIG. 9B is a top cross-sectional view of the fluid platform of FIG. 9A.

FIG. 10A is a top view of an impingement ring according to some embodiments.

FIG. 10B is a top view of an impingement ring according to some embodiments.

FIG. 10C is a top view of an impingement ring according to some embodiments.

FIG. 10D is a top view of an impingement ring according to some embodiments.

FIG. 10E is a top view of an impingement ring according to some embodiments.

FIG. 10F is a top perspective view of an impingement ring according to some embodiments.

FIG. 10G is a top view of an impingement ring according to some embodiments.

FIG. 10H is a top view of an impingement ring according to some embodiments.

FIG. 10I is a top view of an impingement ring according to some embodiments.

FIG. 10J is a perspective view of an impingement ring according to some embodiments.

FIG. 11A is a top perspective view of a fluid platform according to some embodiments.

FIG. 11B is a bottom perspective view of the fluid platform of FIG. 11A.

FIG. 11C is a top perspective exploded view of the fluid platform of FIG. 11A.

FIG. 11D is a side cross-sectional view of the fluid platform of FIG. 11A.

FIG. 11E is a rear cross-sectional view of the fluid platform of FIG. 11A.

FIG. 11F is a top perspective sectional view of the fluid platform of FIG. 11A.

FIG. 11G is a rear view of the fluid platform of FIG. 11A.

FIG. 11H is a front view of the fluid platform of FIG. 11A.

FIG. 11I is a top view of the fluid platform of FIG. 11A.

FIG. 11J is a bottom view of the fluid platform of FIG. 11A.

FIG. 11K is a side cross-sectional view of the fluid platform of FIG. 11A.

FIG. 12A is a top perspective view of a treatment instrument according to some embodiments.

FIG. 12B is a bottom perspective view of the treatment instrument of FIG. 12A.

FIG. 12C is a top perspective exploded view of the treatment instrument of FIG. 12A.

FIG. 12D is a side cross-sectional view of the treatment instrument of FIG. 12A.

FIG. 12E is a magnified bottom perspective sectional view of the fluid platform of the treatment instrument of FIG. 12A.

FIG. 13 is a top perspective sectional view of a fluid platform according to some embodiments.

FIG. 14 is a top perspective sectional view of a fluid platform according to some embodiments.

FIG. 15 is a top perspective sectional view of a fluid platform according to some embodiments.

FIG. 16 is a top perspective sectional view of a fluid platform according to some embodiments.

FIG. 17 is a perspective view of an impingement ring according to some embodiments.

FIG. 18 is a bottom perspective sectional view of a fluid platform according to some embodiments.

FIG. 19 is a bottom perspective sectional view of a fluid platform according to some embodiments.

FIG. 20 is a top perspective sectional view of a fluid platform according to some embodiments.

FIG. 21 is a bottom perspective view of an impingement ring in a fluid platform according to some embodiments.

FIG. 22 is a bottom perspective sectional view of a fluid platform according to some embodiments.

FIG. 23 is a bottom perspective sectional view of a fluid platform according to some embodiments.

FIG. 24 is a side sectional view of a bottom cap of a fluid platform according to some embodiments.

FIG. 25 is a top perspective view of an impingement ring according to some embodiments.

FIG. 26 is a bottom perspective sectional view of a fluid platform according to some embodiments.

FIG. 27 is a top perspective sectional view of a fluid platform according to some embodiments.

FIG. 28 is a bottom perspective sectional view of a fluid platform according to some embodiments.

FIG. 29 is a bottom perspective view of a bottom cap according to some embodiments.

FIG. 30 is a bottom perspective sectional view of a fluid platform according to some embodiments.

FIG. 31 is a bottom perspective sectional view of a fluid platform according to some embodiments.

FIG. 32 is a bottom perspective sectional view of a fluid platform according to some embodiments.

FIG. 33 is a bottom perspective sectional view of a fluid platform according to some embodiments.

FIG. 34 is a bottom perspective sectional view of a fluid platform according to some embodiments.

FIG. 35 is a bottom perspective sectional view of a fluid platform according to some embodiments.

FIG. 36 is a bottom perspective sectional view of a fluid platform according to some embodiments.

FIG. 37 is a bottom perspective sectional view of a fluid platform according to some embodiments.

FIG. 38 is a bottom perspective sectional view of a fluid platform according to some embodiments.

FIG. 39A is a top perspective view of a matrix according to some embodiments.

FIG. 39B is a bottom perspective view of the matrix of FIG. 39A.

FIG. 39C is a front view of the matrix of FIG. 39A.

FIG. 39D is a side view of the matrix of FIG. 39A.

FIG. 39E is a top perspective sectional view of the matrix of FIG. 39A.

FIG. 39F is a top view of the matrix of FIG. 39A.

FIG. 39G is a bottom view of the matrix of FIG. 39A.

FIG. 39H is a rear view of the matrix of FIG. 39A.

FIG. 39I is a side view of the matrix of FIG. 39A showing the opposite side of FIG. 39D.

FIG. 40A is a top perspective view of a matrix according to some embodiments.

FIG. 40B is a top perspective sectional view of the matrix of FIG. 40A.

FIG. 40C is a bottom perspective view of the matrix of FIG. 40A.

FIG. 40D is a front view of the matrix of FIG. 40A.

FIG. 40E a side view of the matrix of FIG. 40A.

FIG. 40F is a top view of the matrix of FIG. 40A.

FIG. 40G is a bottom view of the matrix of FIG. 40A.

FIG. 40H is a rear view of the matrix of FIG. 40.

FIG. 40I is a side view of the matrix of FIG. 40A showing the opposite side of FIG. 40E.

FIG. 41A is a top perspective view of a matrix according to some embodiments.

FIG. 41B is a top perspective sectional view of the matrix of FIG. 41A.

FIG. 41C is a bottom perspective view of the matrix of FIG. 41A.

FIG. 41D is a front view of the matrix of FIG. 41A.

FIG. 41E a side view of the matrix of FIG. 41A.

FIG. 41F is a top view of the matrix of FIG. 41A.

FIG. 41G is a bottom view of the matrix of FIG. 41A.

FIG. 41H is a rear view of the matrix of FIG. 41.

FIG. 41I is a side view of the matrix of FIG. 41A showing the opposite side of FIG. 41E.

FIGS. 42A-42H show aspects of a process for treating a tooth according to some embodiments.

FIG. 43 shows an exemplary process of treating dental caries.

FIG. 44A shows an exemplary pre-treatment image of dental caries.

FIG. 44B shows an exemplary post-treatment image of dental caries.

FIG. 44C shows an exemplary post-treatment image of dental caries.

FIG. 45A shows an exemplary before-treatment image of dental caries from a control group.

FIG. 45B shows an exemplary post-treatment image of treating dental caries from a control group.

FIG. 46A shows exemplary images of treating dental caries on an anterior tooth.

FIG. 46B shows exemplary images of treating dental caries on a premolar.

FIG. 46C shows exemplary images of treating dental caries on a molar.

FIG. 46D shows exemplary images of treating dental caries on an anterior tooth.

FIG. 46E shows exemplary images of treating dental caries on a premolar.

FIG. 46F shows an exemplary image of treating dental caries on a molar.

FIG. 47 shows an exemplary system for treating dental caries.

FIG. 48 shows a schematic system diagram for treating dental caries

FIG. 49A is a top perspective view of a matrix according to some embodiments.

FIG. 49B is a top perspective sectional view of the matrix of FIG. 49A.

FIG. 49C is a bottom perspective view of the matrix of FIG. 49A.

FIG. 49D is a front view of the matrix of FIG. 49A.

FIG. 49E is a top view of the matrix of FIG. 49A.

FIG. 49F is a bottom view of the matrix of FIG. 49A.

FIG. 49G is an exploded view of the matrix of FIG. 49A attached to a treatment instrument.

FIG. 49H is a cross sectional view of the matrix of FIG. 49A attached to a treatment instrument.

FIG. 50A is a top perspective view of a matrix according to some embodiments.

FIG. 50B is a top perspective sectional view of the matrix of FIG. 50A.

FIG. 50C is a bottom perspective view of the matrix of FIG. 50A.

FIG. 50D is a front view of the matrix of FIG. 50A.

FIG. 50E is a top view of the matrix of FIG. 50A.

FIG. 50F is a bottom view of the matrix of FIG. 50A.

FIG. 50G is an exploded view of the matrix of FIG. 50A attached to a treatment instrument.

FIG. 50H is a cross sectional view of the matrix of FIG. 50A attached to a treatment instrument.

Throughout the drawings, unless otherwise noted, reference numbers may be re-used to indicate a general correspondence between referenced elements. The drawings are provided to illustrate example embodiments described herein and are not intended to limit the scope of the disclosure.

### DETAILED DESCRIPTION

Various embodiments disclosed herein relate to a dental treatment instrument configured to clean and/or fill a treatment region of a tooth. The treatment instruments disclosed herein demonstrate improved efficacy at cleaning the tooth, including root canal spaces and associated tubules and carious regions on an exterior surface of the tooth. Additionally or alternatively, the treatment instruments disclosed herein can be used to fill a treatment region of a tooth, such as a treated root canal or a treated carious region on an exterior surface of the tooth.

### Overview of Various Disclosed Embodiments

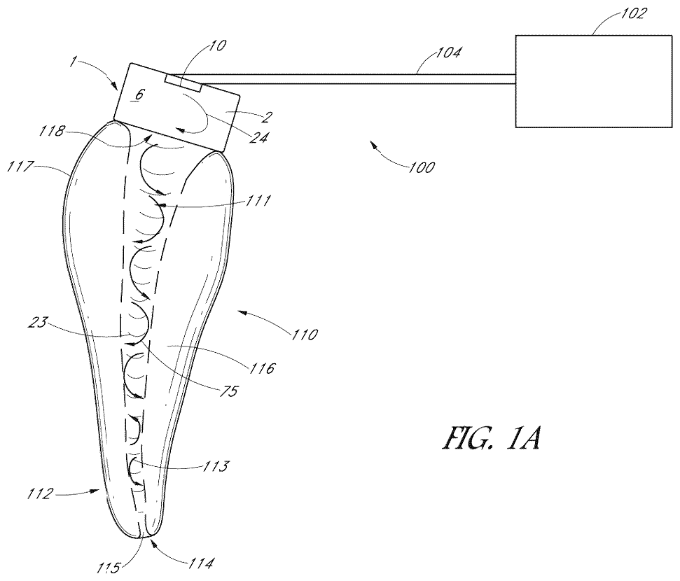

FIG. 1A is a schematic diagram of a system 100 that includes components capable of removing unhealthy or undesirable materials from a tooth 110. The tooth 110 illustrated in FIG. 1A is a premolar tooth, e.g., a tooth located between canine and molar teeth in a mammal such as a human. Although the illustrated tooth 110 comprises a premolar tooth, it should be appreciated that the tooth 110 to be treated can be any type of tooth, such as a molar tooth or an anterior tooth (e.g., an incisor or canine tooth). The tooth 110 includes hard structural and protective layers, including a hard layer of dentin 116 and a very hard outer layer of enamel 117. A pulp cavity 111 is defined within the dentin 116. The pulp cavity 111 comprises one or more root canals 113 extending toward an apex 114 of each root 112. The pulp cavity 111 and root canal 113 contain dental pulp, which is a soft, vascular tissue comprising nerves, blood vessels, connective tissue, odontoblasts, and other tissue and cellular components. Blood vessels and nerves enter/exit the root canal 113 through a tiny opening, the apical foramen or apical opening 115, near a tip of the apex 114 of the root 112. It should be appreciated that, although the tooth 110 illustrated herein is a premolar, the embodiments disclosed herein can advantageously be used to treat any suitable type of tooth, including molars, canines, incisors, etc.

As illustrated in FIG. 1A, the system 100 can be used to remove unhealthy materials (such as organic and inorganic matter) from an interior of the tooth 110, e.g., from the root canal 113 of the tooth 110. For example, an endodontic access opening 118 can be formed in the tooth 110, e.g., on an occlusal surface, or on a side surface such as a buccal surface or a lingual surface. The access opening 118 provides access to a portion of a pulp cavity 111 of the tooth 110. The system 100 can include a console 102 and a treatment instrument 1 comprising a pressure wave generator 10 and a fluid platform 2 adapted to be positioned over or against a treatment region of the tooth 110. The fluid platform 2 can define a chamber 6 configured to retain fluid therein. In some embodiments, the fluid platform 2 can be part of a removable tip device that is removably coupled to a handpiece which can be held or pressed against the tooth 110 by the clinician. In other embodiments, the fluid platform 2 may not be removably connected to the handpiece, e.g., the fluid platform 2 may be integrally formed with the handpiece, or may be connected to the handpiece in a manner intended to be non-removable. In some embodiments, the fluid platform 2 can be attached to the tooth, e.g., using an adhesive. For example, in some embodiments, the fluid platform 2 may not be used with a handpiece. One or more conduits 104 can electrically, mechanically, and/or fluidly connect the console 102 with the fluid platform 2 and pressure wave generator 10. The console 102 can include a control system and various fluid management systems configured to operate the pressure wave generator 10 during a treatment procedure. Additional examples of system components that can be used in the system 100 are disclosed throughout U.S. Pat. No. 9,504,536, the entire contents of which are incorporated by reference herein in their entirety and for all purposes.

As explained herein, the system 100 can be used in cleaning procedures to clean substantially the entire root canal system. For example, in various embodiments disclosed herein, the pressure wave generator 10 can generate pressure waves with a single frequency or multiple frequencies. The single frequency may be a low frequency below the audible range, a frequency within the audible range, or a relatively higher frequency above the audible range. For example, in various embodiments disclosed herein, the pressure wave generator 10 can generate pressure waves 23 of sufficient power and relatively low frequencies to produce fluid motion 24 in the chamber 6—such that the pressure wave generators 10 disclosed herein can act as a fluid motion generator—and can generate pressure waves of sufficient power and at relatively higher frequencies to produce surface effect cavitation on a dental surface, either inside or outside the tooth. That is, for example, the pressure wave generators 10 disclosed herein can act as fluid motion generators to generate large-scale or bulk fluid motion 24 in or near the tooth 110, and can also generate smaller-scale fluid motion at higher frequencies. In some arrangements, the fluid motion 24 in the chamber 6 can generate induced fluid motion such as vortices 75, swirl, a chaotic or turbulent flow, etc. in the tooth 110 and root canal 113 that can clean and/or fill the canal 113.

In some embodiments, the system 100 can additionally or alternatively be used in filling procedures to fill a treated region of the tooth, e.g., to obturate a treated root canal system. The treatment instrument 1 can generate pressure waves and fluid motion that can cause a flowable filling material to substantially fill the treated region. The flowable filling material can be hardened to restore the tooth. Additional details of systems that utilize pressure wave generators 10 to fill a treatment region can be found throughout U.S. Pat. No. 9,877,801, the entire contents of which are hereby incorporated by reference herein in their entirety and for all purposes.

FIG. 1B is a schematic diagram of a system 100 that includes components capable of removing unhealthy or undesirable material from a treatment region on an exterior surface 119 of the tooth. For example, as in FIG. 1A, the system 100 can include a treatment instrument 1 including a fluid platform 2 and a pressure wave generator 10. The fluid platform 2 can communicate with the console 102 by way of the one or more conduits 104. Unlike the system 100 of FIG. 1A, however, the fluid platform 2 is coupled to a treatment region on an exterior surface 119 of the tooth 110. For example, the system 1 of FIG. 1B can be activated to clean an exterior surface of the tooth 110, e.g., a carious region of the tooth 110. In such embodiments, the clinician can provide the chamber 6 over any surface or region of the tooth 110 that includes diseased tissue to provide fluid communication between the pressure wave generator 10 and the treatment region. As with the embodiment of FIG. 1A, fluid motion 24 can be generated in the fluid platform 2 and chamber 6, which can act to clean the treatment region of the tooth 110. Further, as explained above, the system 100 can additionally or alternatively be used to fill the treatment region, e.g., the treated carious region on the exterior surface 119 of the tooth 110.

As explained herein, the disclosed pressure wave generators 10 can be configured to generate pressure waves 23 with energy sufficient to clean undesirable material from a tooth. The pressure wave generator 10 can be a device that converts one form of energy into pressure waves 23 within the treatment liquid. The pressure wave generator 10 can induce, among other phenomena, fluid dynamic motion of the treatment liquid (e.g., in the chamber 6), fluid circulation, turbulence, and other conditions that can enable the cleaning of the tooth 110. The pressure wave generators 10 disclosed in each of the figures described herein may be any suitable type of pressure wave generator.

The pressure wave generator 10 can be used to clean the tooth 110 by creating pressure waves 23 that propagate through the treatment liquid, e.g., through treatment fluid retained at least partially retained in the fluid platform 2. In some implementations, the pressure wave generator 10 may also create cavitation, acoustic streaming, shock waves, turbulence, etc. In various embodiments, the pressure wave generator 10 can generate pressure waves 23 or acoustic energy having a broadband power spectrum. For example, the pressure wave generator 10 can generate acoustic waves at multiple different frequencies, as opposed to only one or a few frequencies. Without being limited by theory, it is believed that the generation of power at multiple frequencies can help to remove various types of organic and/or inorganic materials that have different material or physical characteristics at various frequencies.

In some embodiments, the pressure wave generator 10 can comprise a liquid jet device. The liquid jet can be created by passing high pressure liquid through an orifice. The liquid jet can create pressure waves 23 within the treatment liquid. In some embodiments, the pressure wave generator 10 comprises a coherent, collimated jet of liquid. The jet of liquid can interact with liquid in a substantially-enclosed volume (e.g., the chamber 6) and/or an impingement member (e.g., a distal impingement plate on a distal end of a guide tube, or a curved surface of the chamber walls) to create the pressure waves 23. As used herein, “member” means a constituent piece, portion, part, component, or section of a structure. In addition, the interaction of the jet and the treatment fluid, as well as the interaction of the spray which results from hitting the impingement member and the treatment fluid, may assist in creating cavitation and/or other acoustic effects to clean the tooth. In other embodiments, the pressure wave generator 10 can comprise a laser device, as explained herein. Other types of pressure wave generators, such as mechanical devices, may also be suitable.

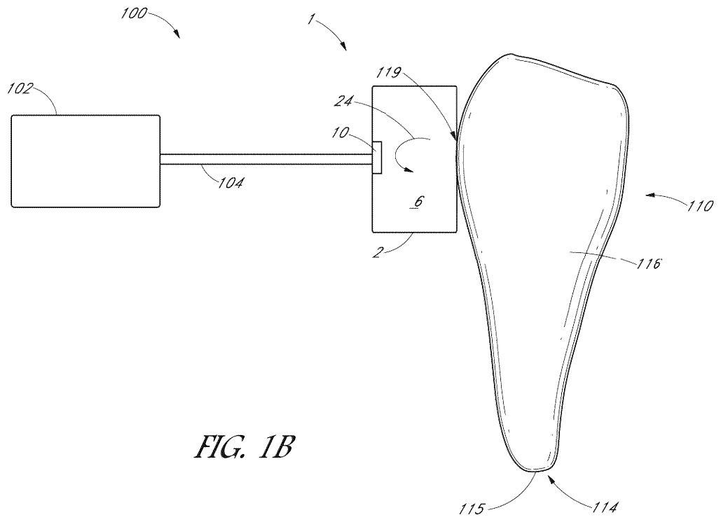

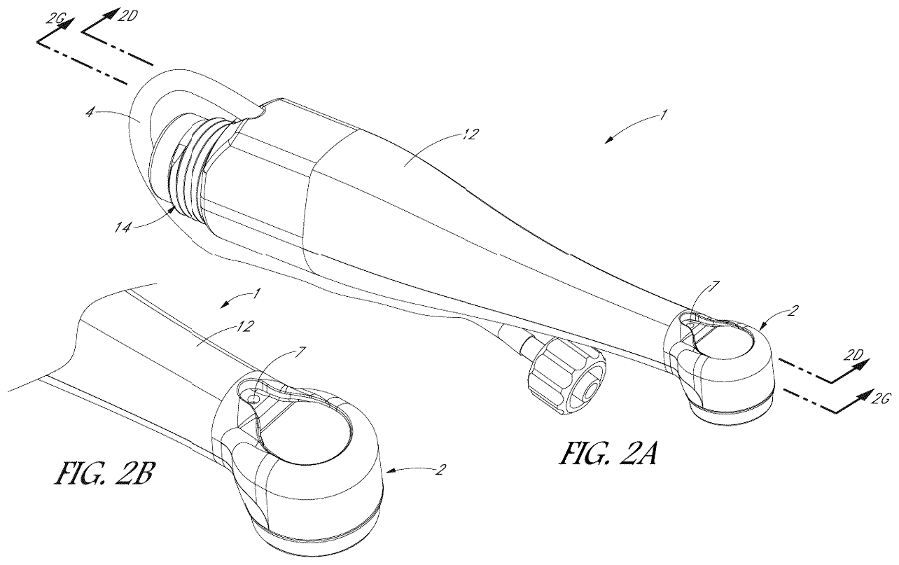

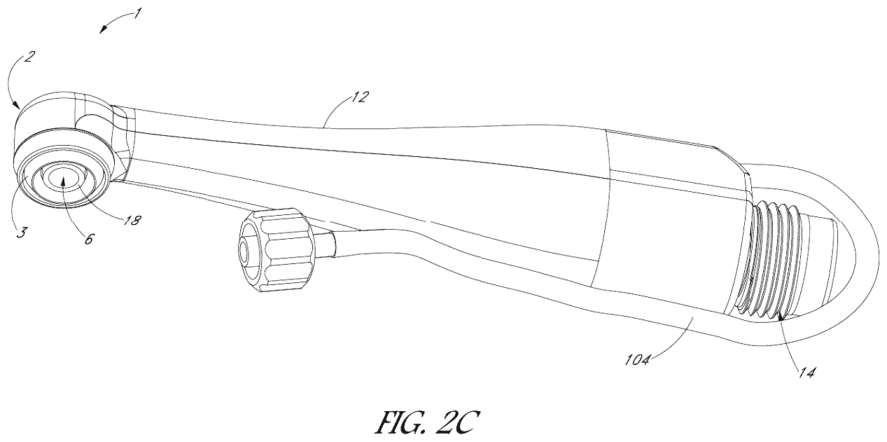

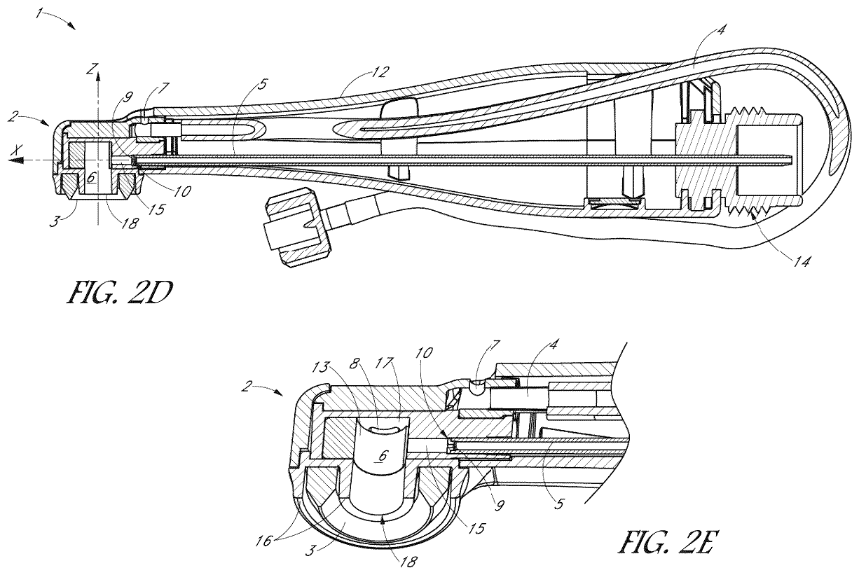

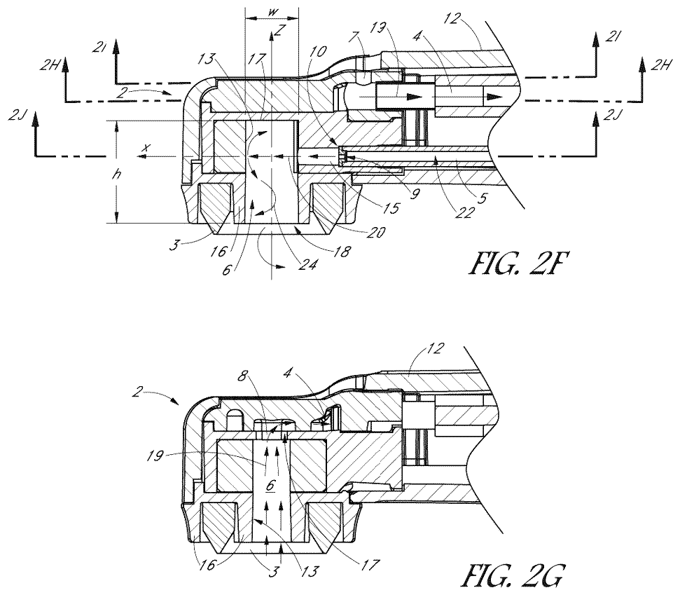

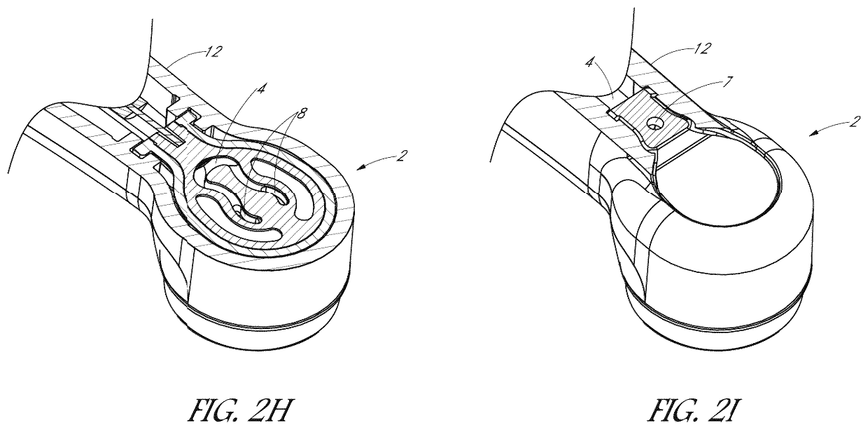

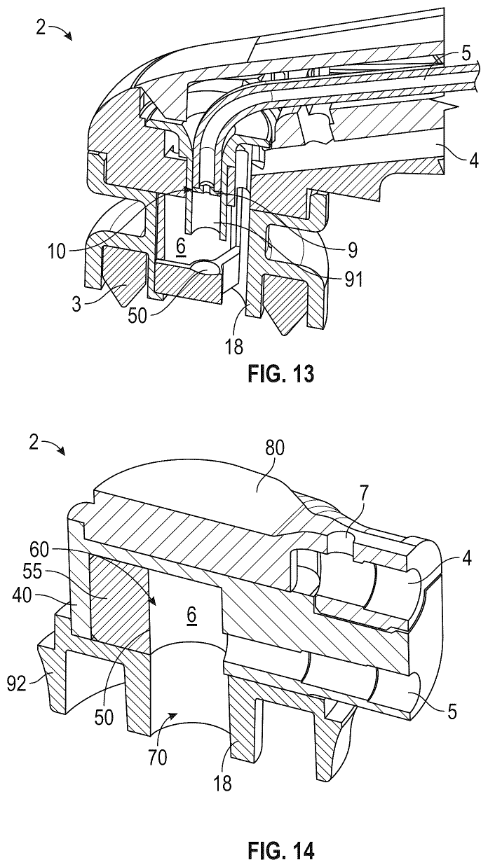

The pressure wave generators 10 disclosed herein can generate pressure waves having a broadband acoustic spectrum with multiple frequencies. The pressure wave generator 10 can generate a broadband power spectrum of acoustic power with significant power extending from about 1 Hz to about 1000 kHz, including, e.g., significant power in a range of about 1 kHz to about 1000 kHz (e.g., the bandwidth can be about 1000 kHz). The bandwidth of the acoustic energy spectrum may, in some cases, be measured in terms of the 3-decibel (3-dB) bandwidth (e.g., the full-width at half-maximum or FWHM of the acoustic power spectrum). In various examples, a broadband acoustic power spectrum can include significant power in a bandwidth in a range from about 1 Hz to about 500 kHz, in a range from about 1 kHz to about 500 kHz, in a range from about 10 kHz to about 100 kHz, or some other range of frequencies. In some implementations, a broadband spectrum can include acoustic power above about 1 MHz. Beneficially, a broadband spectrum of acoustic power can produce a relatively broad range of bubble sizes in the cavitation cloud and on the surfaces on the tooth, and the implosion of these bubbles may be more effective at disrupting tissue than bubbles having a narrow size range. Relatively broadband acoustic power may also allow acoustic energy to work on a range of length scales, e.g., from the cellular scale up to the tissue scale. Accordingly, pressure wave generators that produce a broadband acoustic power spectrum (e.g., some embodiments of a liquid jet) can be more effective at tooth cleaning for some treatments than pressure wave generators that produce a narrowband acoustic power spectrum. Additional examples of pressure wave generators that produce broadband acoustic power are described in FIGS. 2A-2B-2 and the associated disclosure of U.S. Pat. No. 9,675,426, and in FIGS. 13A-14 and the associated disclosure of U.S. Pat. No. 10,098,717, the entire contents of each of which are hereby incorporated by reference herein in their entirety and for all purposes.

The dental treatments disclosed herein can be used with any suitable type of treatment fluid, e.g., cleaning fluids. In filling procedures, the treatment fluid can comprise a flowable filling material that can be hardened to fill the treatment region. The treatment fluids disclosed herein can be any suitable fluid, including, e.g., water, saline, etc. In some embodiments, the treatment fluid can be degassed, which may improve cavitation and/or reduce the presence of gas bubbles in some treatments. In some embodiments, the dissolved gas content can be less than about 1% by volume. Various chemicals can be added to treatment solution, including, e.g., tissue dissolving agents (e.g., NaOCl), disinfectants (e.g., chlorhexidine), anesthesia, fluoride therapy agents, EDTA, citric acid, and any other suitable chemicals. For example, any other antibacterial, decalcifying, disinfecting, mineralizing, or whitening solutions may be used as well. Various solutions may be used in combination at the same time or sequentially at suitable concentrations. In some embodiments, chemicals and the concentrations of the chemicals can be varied throughout the procedure by the clinician and/or by the system to improve patient outcomes. The pressure waves 23 and fluid motion 24 generated by the pressure wave generator 10 can beneficially improve the efficacy of cleaning by inducing low-frequency bulk fluid motion and/or higher-frequency acoustic waves that can remove undesirable materials throughout the treatment region.

In some systems and methods, the treatment fluids used with the system 100 can comprise degassed fluids having a dissolved gas content that is reduced when compared to the normal gas content of the fluid. The use of degassed treatment fluids can beneficially improve cleaning efficacy, since the presence of bubbles in the fluid may impede the propagation of acoustic energy and reduce the effectiveness of cleaning. In some embodiments, the degassed fluid has a dissolved gas content that is reduced to approximately 10%-40% of its normal amount as delivered from a source of fluid (e.g., before degassing). In other embodiments, the dissolved gas content of the degassed fluid can be reduced to approximately 5%-50% or 1%-70% of the normal gas content of the fluid. In some treatments, the dissolved gas content can be less than about 70%, less than about 50%, less than about 40%, less than about 30%, less than about 20%, less than about 10%, less than about 5%, or less than about 1% of the normal gas amount. In some embodiments, the degassed fluids may be exposed to a specific type of gas, such as ozone, and carry some of the gas (e.g., ozone) with them into the treatment region, for example, in the form of gas bubbles. At the treatment region, the gas bubbles expose the treatment region to the gas (e.g., ozone) for further disinfection of the region. Additional details regarding the use of degassed treatment liquids may be found in U.S. Pat. No. 9,675,426, which is incorporated by reference herein in its entirety and for all purposes.

### Examples of Treatment Instruments

Various embodiments disclosed herein relate to a dental treatment instrument 1 configured to clean and/or fill a treatment region of the tooth 110. The treatment instruments disclosed herein demonstrate improved efficacy at cleaning the tooth 110, including root canal spaces and associated tubules and carious regions on an exterior surface of the tooth 110.

FIGS. 2A-2K illustrate an example of such a treatment instrument 1. In particular, FIG. 2A is a schematic perspective view of a treatment instrument 1 according to one embodiment. FIG. 2B is a magnified schematic perspective view of a fluid platform 2 disposed at a distal end portion of a handpiece 12 of the treatment instrument 1 of FIG. 2A. FIG. 2C is a schematic bottom perspective view of the treatment instrument 1 of FIG. 2A. FIG. 2D is a schematic side sectional view of the treatment instrument 1 of FIG. 2A, taken along section 2D-2D of FIG. 2A. FIG. 2E is a magnified bottom perspective sectional view of the fluid platform 2. FIG. 2F is a magnified view of the fluid platform 2 shown in the section of FIG. 2D. FIG. 2G is a schematic side sectional view of the fluid platform 2 taken along section 2G-2G of FIG. 2A. FIG. 2H is a top perspective sectional view of the fluid platform 2 taken along section 2H-2H of FIG. 2F. FIG. 2I is a top perspective sectional view of the fluid platform 2 taken along section 2I-2I of FIG. 2F. FIG. 2J is a top plan view of the fluid platform 2 taken along section 2J-2J of FIG. 2F. FIG. 2K is a top plan view of the fluid platform 2 taken along section 2I-2I of FIG. 2F.

The treatment instrument 1 of FIGS. 2A-2K includes a handpiece 12 sized and shaped to be gripped by the clinician. A fluid platform 2 can be coupled to a distal portion of the handpiece 12. As explained herein, in some embodiments, the fluid platform 2 can form part of a removable tip device 11 (see below) that can be removably connected to the handpiece 12. In other embodiments, the fluid platform 2 can be non-removably attached to the handpiece 12 or can be integrally formed with the handpiece 12. In still other embodiments, the fluid platform 2 may not couple to a handpiece and may instead serve as a treatment cap that is adhered (or otherwise coupled or positioned) to the tooth without using a handpiece. As shown in FIG. 2A, an interface member 14 can be provided at a proximal end portion of the handpiece 12, which can removably couple to the one or more conduits 104 to provide fluid communication between the console 102 and the treatment instrument 1.

As shown in FIGS. 2A-2B, and as explained herein, a vent 7 can be provided through a portion of the handpiece 12 to provide fluid communication between an outlet line 4 (which can comprise one of the at least one conduits 104 described above) and ambient air. As explained herein, the vent 7 can serve to regulate the pressure in the fluid platform 2 and can improve the safety and efficacy of the treatment instrument 1. As shown in FIG. 2C, an access port 18 can be provided at a distal portion of the fluid platform 2 to provide fluid communication between a chamber 6 defined by the fluid platform 2 and the treatment region of the tooth 110. For example, as explained above with respect to FIG. 1A, in root canal cleaning procedures, a sealing cap 3 at the distal portion of the fluid platform 2 can be positioned against the tooth 110 over the access opening 118 to provide fluid communication between the chamber 6 and the interior of the tooth 110 (e.g., the pulp cavity 111 and root canal(s) 113). In other embodiments, as explained above with respect to FIG. 1B, the sealing cap 3 can be positioned against the tooth 110 over the carious region at an exterior surface 119 of the tooth 110 to provide fluid communication between the chamber 6 and the carious region to be treated. The pressure waves 23 and fluid motion 24 can propagate throughout the treatment region to clean the treatment region.

Turning to FIGS. 2D-2G, the fluid platform 2 can have one or a plurality of walls that define the chamber 6. For example, as shown in FIGS. 2E-2G the fluid platform 2 can comprise at least one wall including a curved sidewall 13 and an upper wall 17 disposed at an upper end of the chamber 6 opposite the access port 18. In the illustrated embodiment, the curved sidewall 13 can define a generally cylindrical chamber 6 with a generally circular cross-section, and can extend from the upper wall 17 at an angle. In other embodiments, however, the curved sidewall 13 can be elliptical or can have other curved or angular surfaces. The sidewall 13 can extend non-parallel to (e.g., substantially transverse to) the upper wall 17. The sidewall 13 can extend from the upper wall 17 at any suitable non-zero angle, for example, by about 90° in some embodiments. In other embodiments, the sidewall 13 can extend from the upper wall 17 by an angle greater than or less than 90°. In other embodiments, the sidewall 13 can extend from the upper wall 17 by different angular amounts along a perimeter of the sidewall 13 such that the shape of the chamber 6 may be irregular or asymmetric. In the illustrated embodiment, the interior angle between the upper wall 17 and sidewall 13 can comprise an angle or corner. In other embodiments, however, the interior interface between the upper wall 17 and sidewall 13 can comprise a curved or smooth surface without corners. For example, in some embodiments, the one or more walls can comprise a curved profile, such as a quasi-spherical profile.

The sealing cap 3 can be coupled or formed with the fluid platform 2. As shown, for example, a flange 16 can comprise a U-shaped support with opposing sides, and the sealing cap 3 can be disposed within the flange 16. The flange 16 can serve to mechanically connect the sealing cap 3 to the distal portion of the handpiece 12. The access port 18 can be provided at the distal end portion of the chamber 6 which places the chamber 6 in fluid communication with a treatment region of the tooth 110 when the chamber 6 is coupled to the tooth (e.g., pressed against the tooth, adhered to the tooth, or otherwise coupled to the tooth). For example, the sealing cap 3 can be pressed against the tooth by the clinician to substantially seal the treatment region of the tooth.

The chamber 6 can be shaped to have any suitable profile. In various embodiments, and as shown, the chamber 6 can have a curved sidewall 13, but in other embodiments, the chamber 6 can have a plurality of angled sidewalls 13 that may form angled interior corners. The sectional plan view (e.g., bottom sectional view) of the chamber 6 can accordingly be rounded, e.g., generally circular as shown in, e.g., FIGS. 2C and 2J. In some embodiments, the sectional plan view (e.g., bottom sectional view) of the chamber 6 can be elliptical, polygonal, or can have an irregular boundary.

The chamber 6 can have a central axis Z. For example, as shown in FIG. 2D, the central axis Z can extend substantially transversely through a center (e.g., a geometric center) of the access port 18 (e.g., through a distal-most plane of the chamber 6 defined at least in part by the access port 18). In various embodiments, and as shown in FIG. 2D, for a chamber 6 with a circular (or approximately circular) cross-section (as viewed from a bottom plan view) the central axis Z can pass substantially transversely through the approximate center of the access port 18 that at least partially defines a distal portion of the chamber 6 and/or the upper wall 17 that at least partially defines the top of the chamber 6. For example, the central axis Z can pass substantially transversely through the geometric center of the upper wall 17 and/or the access port 18 at an angle in a range of 85° to 95°, at an angle in a range of 89° to 91°, or at an angle in a range of 89.5° to 90.5°.

As explained above, although the illustrated chamber 6 has a generally or approximately circular cross-section, the chamber 6 may have other suitable shapes as viewed in various bottom-up cross-sections. In such embodiments, a plurality of planes (e.g., two, three, or more planes) parallel to the plane of the opening of the access port 18 of the chamber 6 (which may be at a distal-most plane of the chamber 6) can be delimited or bounded by the sidewall 13 of the chamber. The central axis Z can pass through the approximate geometric center of each of the bounded planes parallel to the access port 18. For example, the chamber 6 may have a sidewall 13 that is angled non-transversely relative to the upper wall 17, and/or may have a sidewall 13 with a profile that varies along a height h of the chamber 6. The central axis Z can pass through the geometric center of each of the plurality of parallel bounded planes.

A pressure wave generator 10 (which can serve as a fluid motion generator) can be arranged to generate pressure waves and rotational fluid motion in the chamber 6. The pressure wave generator 10 can be disposed outside the tooth during a treatment procedure. The pressure wave generator 10 can comprise a liquid supply port that can deliver a liquid stream (such as a liquid jet) across the chamber 6 (e.g., completely across the chamber 6 to impinge upon a portion of the sidewall 13 opposite the pressure wave generator 10 or supply port) to generate pressure waves and fluid motion. For example, the pressure wave generator 10 can comprise a liquid jet device that includes an orifice or nozzle 9. Pressurized liquid 22 can be transferred to the nozzle 9 along an inlet line 5. The inlet line 5 can be connected to a fluid source in the console 102, for example, by way of the one or more conduits 104. The nozzle 9 can have a diameter selected to form a high velocity, coherent, collimated liquid jet. The nozzle 9 can be positioned at a distal end of the inlet line 5. In various embodiments disclosed herein, the nozzle 9 can have an opening with a diameter in a range of 59 microns to 69 microns, in a range of 60 microns to 64 microns, or in a range of 61 microns to 63 microns. For example, in one embodiment, the nozzle 9 can have an opening with a diameter of approximately 62 microns, which has been found to generate liquid jets that are particularly effective at cleaning teeth. Although the illustrated embodiments are configured to form a liquid jet (e.g., a coherent, collimated jet), in other embodiments, the liquid stream may not comprise a jet but instead a liquid stream in which the momentum of the stream is generally parallel to the stream axis.

As shown in FIGS. 2D and 2F, the nozzle 9 can be configured to direct a liquid stream comprising a liquid jet 20 laterally through a laterally central region of the chamber 6 along a jet axis X (also referred to as a stream axis) non-parallel to (e.g., substantially perpendicular to) the central axis Z. In some embodiments, the jet axis X can intersect the central axis Z. In various embodiments, the liquid stream (e.g., the jet 20) can intersect the central axis Z. In other embodiments, the jet axis X can be slightly offset from the central axis Z. The liquid jet 20 can generate fluid motion 24 (e.g., vortices) that can propagate throughout the treatment region (e.g., throughout a root canal, throughout a carious region on an external surface of the tooth, etc.) to interact with and remove unhealthy material. In some embodiments, the pressure wave generator 10 can generate broadband pressure waves through the fluid in the chamber 6 to clean the treatment region. Additional details regarding jets, such as liquid jet 20, that may be formed by the nozzle 9, are described in U.S. Pat. Nos. 8,753,121, 9,492,244, and 9,675,426, the entire contents of each of which are hereby incorporated by reference herein in their entirety and for all purposes.

As shown in FIGS. 2F and 2J, the nozzle 9 can form the coherent, collimated liquid jet 20, which can pass along a guide channel 15 disposed between the nozzle 9 and the chamber 6. The guide channel 15 may provide improved manufacturability and can serve as a guide for the liquid jet 20 to the chamber 6. During operation, the chamber 6 can fill with the treatment liquid supplied by the liquid jet 20 (and/or additional inlets to the chamber 6). The jet 20 can enter the chamber 6 from the guide channel 15 and can interact with the liquid retained in the chamber 6. The interaction between the liquid jet 20 and the liquid in the chamber 6 can create the fluid motion 24 and/or pressure waves 23 (e.g., shown in FIGS. 1A and 1B), which can propagate throughout the treatment region. The liquid jet 20 can impact the sidewall 13 of the chamber 6 at a location opposite the nozzle 9 along the jet axis X. The sidewall 13 of the chamber 6 can serve as an impingement surface such that, when the jet 20 impinges on or impacts the sidewall 13, the curved or angled surface of the sidewall 13 creates fluid motion along the sidewall 13, the upper wall 17, and/or within the fluid retained in the chamber 6. Moreover, the movement of the jet 20 and/or the liquid stream diverted by the sidewall 13 can induce fluid motion 24 in the chamber 6 and through the treatment region.

Without being limited by theory, for example, directing the jet 20 across the chamber 6 (e.g., completely across the chamber 6) along the jet axis X at a central location within the chamber 6 can induce fluid motion 24 comprising vortices that rotate about an axis non-parallel to (e.g., perpendicular to) the central axis Z of the chamber 6. The vortices can propagate through the treatment region and can provide bulk fluid motion that flushes undesirable material (e.g., decayed organic matter) out of the treatment region. The combination of the vortex fluid motion 24 and the generated pressure waves 23 can effectively remove undesirable materials of all shapes and sizes from large and small spaces, cracks, and crevices of the treatment region. The fluid motion 24 may be turbulent in nature and may rotate about multiple axes, which can increase the chaotic nature of the flow and improve treatment efficacy.

As shown in FIGS. 2G, 2H, and 2K, the treatment instrument 1 can also include an evacuation or outlet line 4 to convey waste or effluent liquids 19 to a waste reservoir, which may be located in the system console 102. A suction port 8 or fluid outlet can be exposed to the chamber 6 along a wall of the chamber 6 offset from the central axis Z. For example, as shown in FIG. 2G, the suction port 8 can be disposed along the upper wall 17 of the chamber 6 opposite the access port 18. A vacuum pump (not shown) can apply vacuum forces along the outlet line 4 to draw waste or effluent liquids 19 out of the chamber 6 through the suction port 8, along the outlet line 4, and to the waste reservoir. In some embodiments, only one suction port 8 can be provided. However, as shown in the embodiment of FIGS. 2H and 2K, the instrument 1 can include a plurality (e.g., two) of suction ports positioned laterally opposite one another. In some embodiments, more than two suction ports can be provided. The suction ports 8 can be disposed laterally opposite one another, e.g., symmetrically relative to, the central axis Z. As shown, the suction ports 8 can be disposed through the upper wall 17 at or near the sidewall 13, e.g., closer to the sidewall 13 than to the central axis Z of the chamber 6. In the illustrated embodiment, the suction ports 8 can abut or be defined at least in part by the sidewall 13. In other embodiments, the suction ports 8 can be laterally inset from the sidewall 13. In still other embodiments, the suction ports 8 can be disposed on the sidewall 13 of the chamber 6.

Accordingly, in various embodiments, the chamber 6 can have a maximum lateral dimension in a first plane extending substantially transverse to (e.g., at an angle in a range of 85° to 95°, at an angle in a range of 89° to 91°, or at an angle in a range of 89.5° to 90.5° relative to) the central axis Z. The first plane can be delimited by a wall of the chamber along a boundary of the wall. A projection of the suction port 8 onto the first plane can be closer to the boundary than to the central axis Z of the chamber 6. For example, in the illustrated embodiment, the chamber 6 can comprise an approximately circular bottom cross-section, and the first plane substantially transverse to the central axis Z can be delimited along the sidewall 13 by an approximately circular boundary. A projection of the suction port 8 onto that first plane can be closer to the approximately circular boundary than to the central axis Z.

As shown, the suction ports 8 can comprise elongated and curved (e.g. kidney-shaped) openings. The curvature of the suction ports 8 may generally conform to the curvature of the sidewall 13 of the chamber 6 in some embodiments. In other embodiments, the suction ports 8 may not be curved but may be polygonal (e.g., rectangular). Beneficially, the use of an elongate suction port 8, in which a length of the opening is larger than a width, can prevent large particles from clogging the suction port 8 and/or outlet line 4. In some embodiments, the suction port 8 can comprise an opening flush with the upper wall 17. In other embodiments, the suction port 8 can protrude partially into the chamber 6.

In some embodiments, pressure wave generator 10 and the suction port(s) 8 can be shaped and positioned relative to the chamber 6 such that, during operation of the treatment instrument 1 in a treatment procedure, pressure at a treatment region of the tooth (e.g., within the root canals of the tooth as measured in the apex) can be maintained within a range of 50 mmHg to −500 mmHg. Maintaining the pressure at the treatment region within desired ranges can reduce the risk of pain to the patient, prevent extrusion of liquids apically out of the apical opening 115, and/or improve cleaning efficacy. For example, the pressure wave generator 10 and the suction port(s) 8 can be shaped and positioned relative to the chamber 6 such that, during operation of the treatment instrument 1 in a treatment procedure, apical pressure at or near the apex 114 and apical opening 115 are maintained at less than 50 mmHg, at less than 5 mmHg, at less than −5 mmHg, e.g., within a range of −5 mmHg to −200 mmHg, within a range of −5 mmHg to −55 mmHg, or within a range of −10 mmHg to −50 mmHg. Maintaining the apical pressure within these ranges can reduce the risk of pain to the patient, prevent extrusion of liquids apically out of the apical opening 115, and/or improve cleaning efficacy.

In some embodiments, to regulate apical pressure, the suction ports 8 can be circumferentially offset from the nozzle 9. For example, in the illustrated embodiment, the suction ports 8 can be circumferentially offset from the nozzle 9 by about 90°.

Further, the chamber 6 can have a width w (e.g., a diameter or other major lateral dimension of the chamber 6) and a height h extending from the upper wall 17 to the access port 18. The width w and height h can be selected to provide effective cleaning outcomes while maintaining apical pressure in desired ranges. In various embodiments, for example, the width w of the chamber 6 can be in a range of 2 mm to 4 mm, in a range of 2.5 mm to 3.5 mm, or in a range of 2.75 mm to 3.25 mm (e.g., about 3 mm). A height h of the chamber 6 can be in a range of about 1 mm to 30 mm, in a range of about 2 mm to 10 mm, or in a range of about 3 mm to 5 mm.

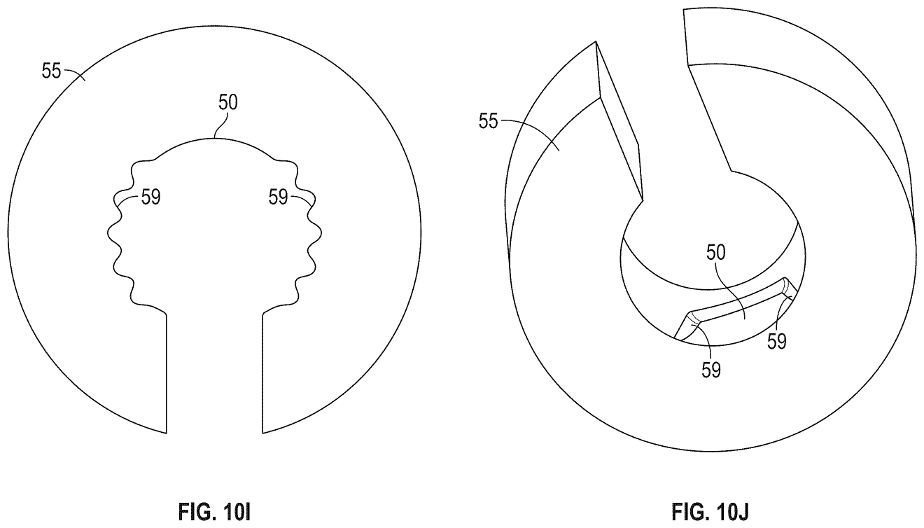

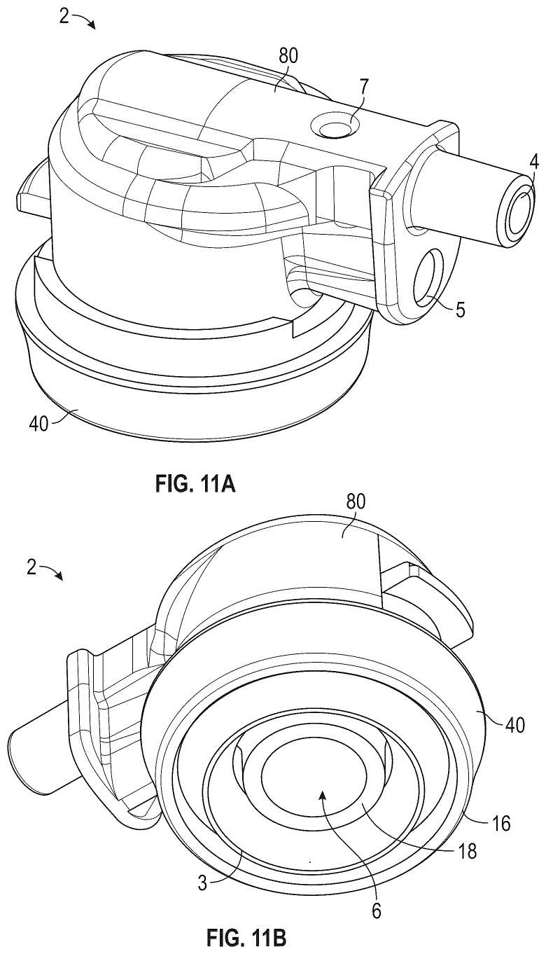

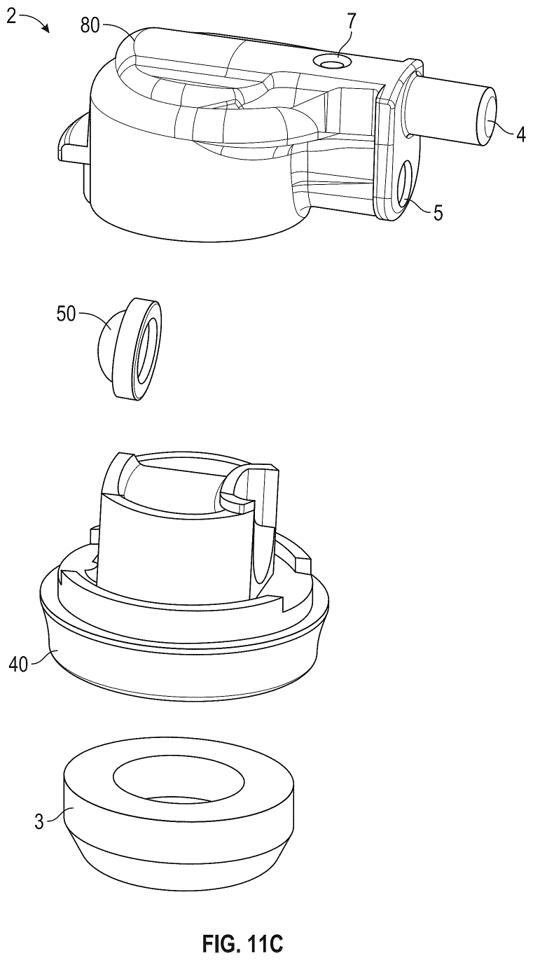

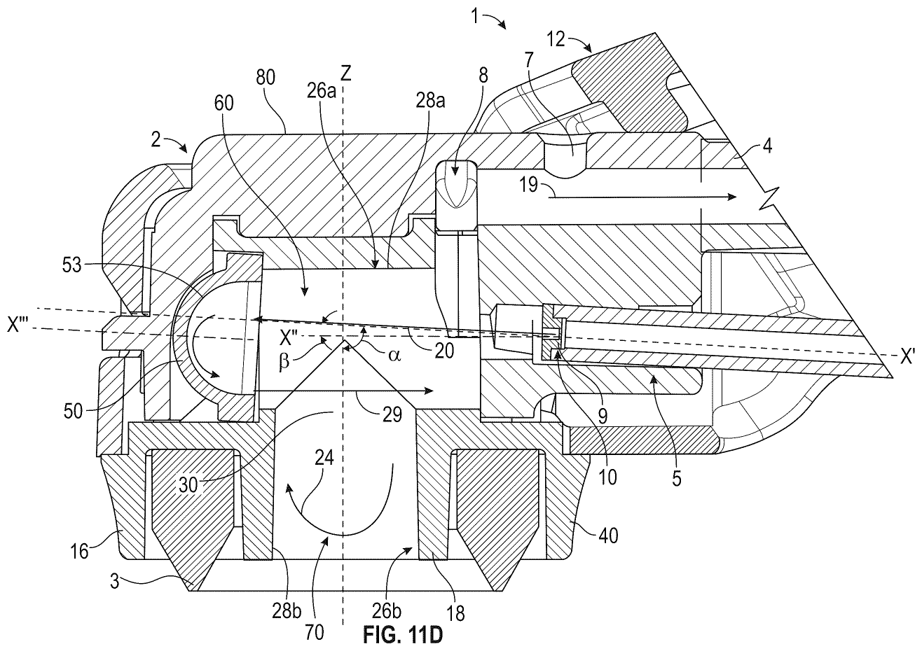

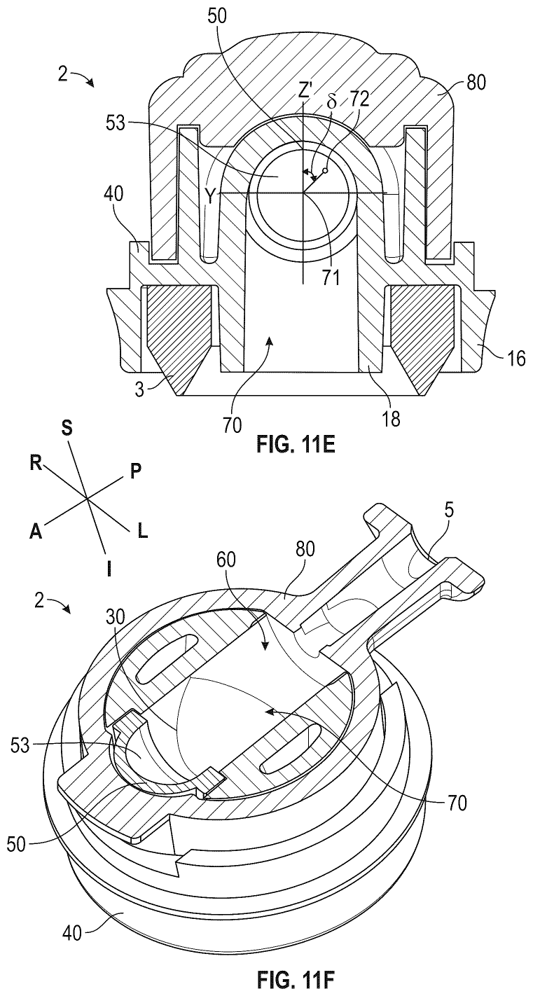

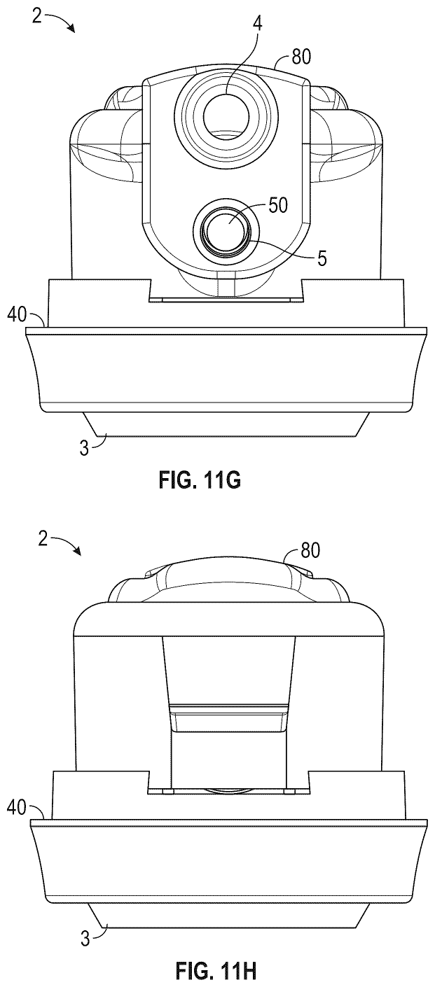

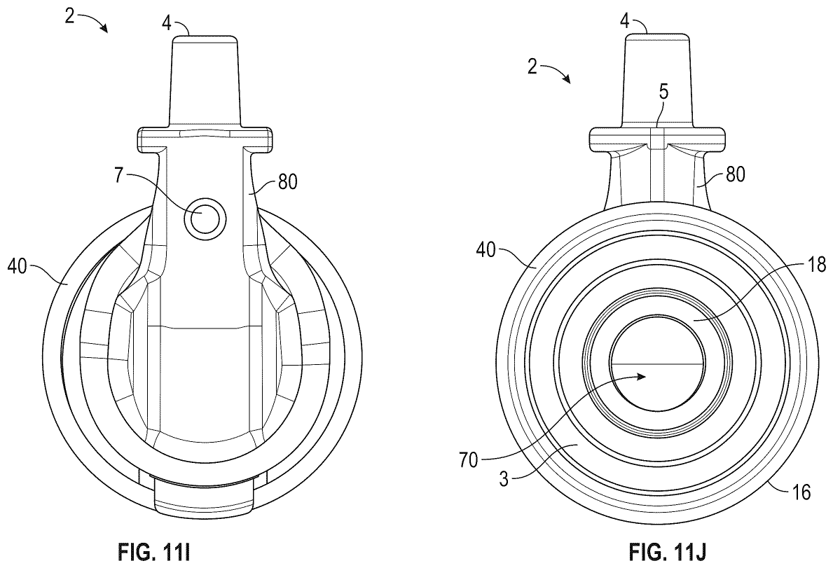

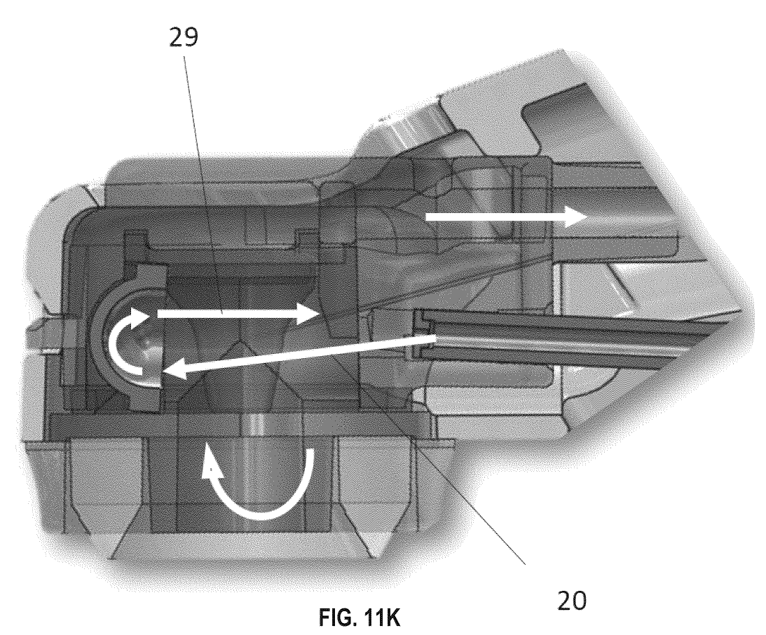

The pressure wave generator 10 (e.g., the nozzle 9) can be positioned relative to the chamber 6 at a location that generates sufficient fluid motion 24 to treat the tooth. As shown, the pressure wave generator 10 (including, e.g., the nozzle 9) can be disposed outside the chamber 6 as shown (for example, recessed from the chamber 6). In some embodiments, the pressure wave generator 10 can be exposed to (or flush with) the chamber 6 but may not extend into the chamber 6. In still other embodiments, at least a portion of the pressure wave generator 10 may extend into the chamber 6. The pressure wave generator 10 (for example, including the nozzle 9) can be positioned below or distal the suction ports 8. Moreover, in the illustrated embodiment, the jet 20 can be directed substantially perpendicular to the central axis Z (such that an angle between the jet axis X and the central axis Z is approximately 90°). In other embodiments, as described, for example, with respect to FIGS. 11A-11J, the jet can be directed at a non-perpendicular angle to the central axis Z. The jet 20 can pass proximate the central axis Z of the chamber, e.g., pass through a laterally central region of the chamber 6. For example, in some embodiments, the jet axis X or the liquid jet 20 can intersect the central axis Z of the chamber. In some embodiments, the jet 20 may pass through a laterally central region of the chamber 6 but may be slightly offset from the central axis Z. For example, the central axis Z can lie in a second plane that is substantially transverse to the jet axis X (e.g., the second plane can be angled relative to the jet axis X in a range of 85° to 95°, in a range of 89° to 91°, or in a range of 89.5° to 90.5°). The stream or jet axis X can intersect the second substantially transverse plane at a location closer to the central axis Z than to the sidewall 13.

Accordingly, as explained above, the chamber 6 can have a maximum lateral dimension in a first plane extending substantially transverse to the central axis Z, and the central axis Z can lie in the second plane extending substantially transverse to the stream or jet axis X. The first plane can be delimited by a wall (for example, the sidewall 13) of the chamber 6 along a boundary of the wall. As explained above, the suction port 8 can be closer to the boundary (e.g., the sidewall 13 in some embodiments) than to the central axis Z. The suction port 8 may also be closer to the boundary than to the location at which the stream or jet axis X intersects the second plane. Further, the location at which the stream or jet axis X intersects the second plane can be closer to the central axis Z than to the suction port 8 (or to a projection of the suction port 8 onto that second plane). Although the wall illustrated herein can comprise an upper wall and sidewall extending therefrom, in other embodiments, the wall can comprise a single curved wall, or can have any other suitable shape.

As explained above, the vent 7 can be provided through the platform 2 and can be exposed to ambient air. The vent 7 can be in fluid communication with the evacuation line 4 that is fluidly connected to the suction port 8. The vent 7 can be disposed along the evacuation or outlet line 4 at a location downstream of the suction port 8. The vent 7 can beneficially prevent or reduce over-pressurization in the chamber 6 and treatment region. For example, ambient air from the outside environs can be entrained with the effluent liquid 19 removed along the outlet line 4. The vent 7 can regulate pressure within the treatment region by allowing the application of a static negative pressure. For example, a size of the vent 7 can be selected to provide a desired amount of static negative pressure at the treatment region. The vent 7 can be positioned at a location along the outlet line 4 so as to prevent ambient air from entering the chamber 6 and/or the treatment region of the tooth 110. Additional details regarding vented fluid platforms can be found throughout U.S. Pat. No. 9,675,426, the entire contents of which are incorporated by reference herein in their entirety and for all purposes.

Beneficially, the embodiment of FIGS. 2A-2K and like embodiments can create sufficient fluid motion and pressure waves to provide a thorough cleaning of the entire treatment region. Components such as the pressure wave generator 10, the chamber 6, the suction port 10, the vent 7, etc. can be arranged as shown and described in the illustrated embodiment, so as to provide effective treatment (e.g., effective cleaning or filling), improved pressure regulation (e.g., maintain pressures at the treatment region within suitable ranges), and improved patient outcomes as compared with other devices.

The embodiments of the treatment instrument 1 disclosed herein can be used in combination with the features shown and described throughout U.S. Pat. No. 10,363,120, the entire contents of which are incorporated by reference herein in its entirety and for all purposes.

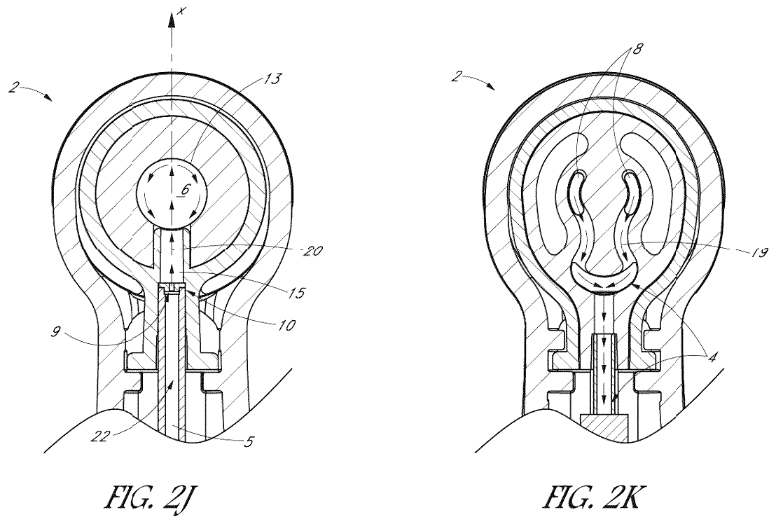

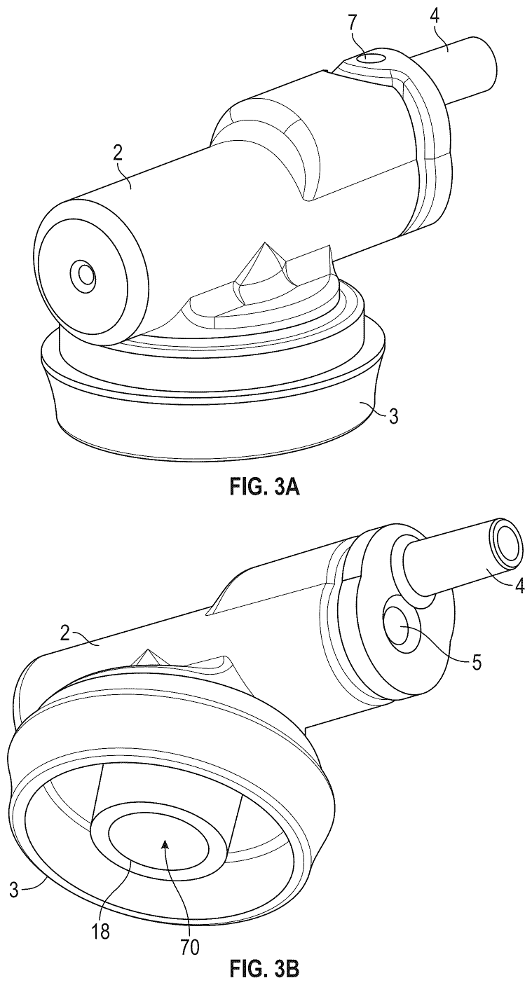

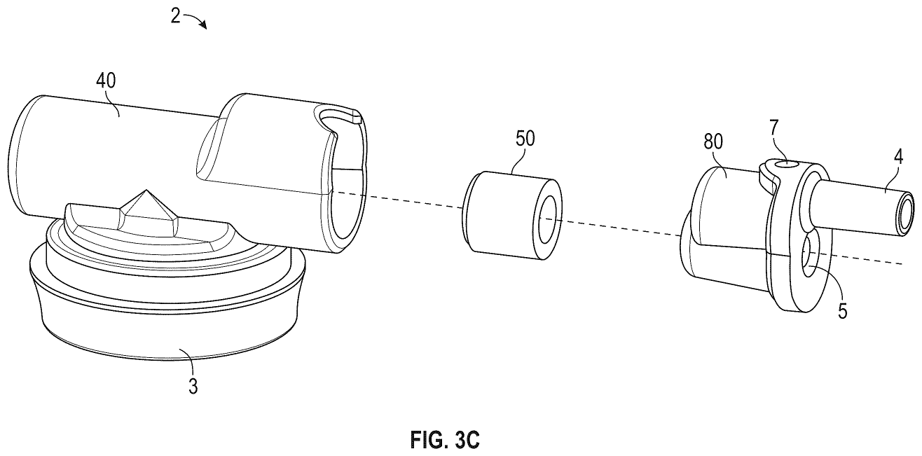

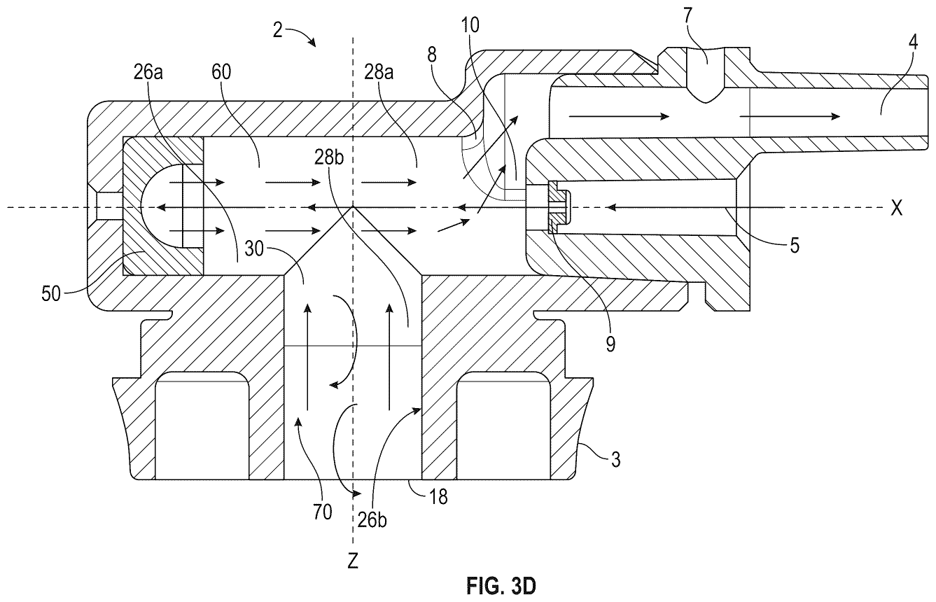

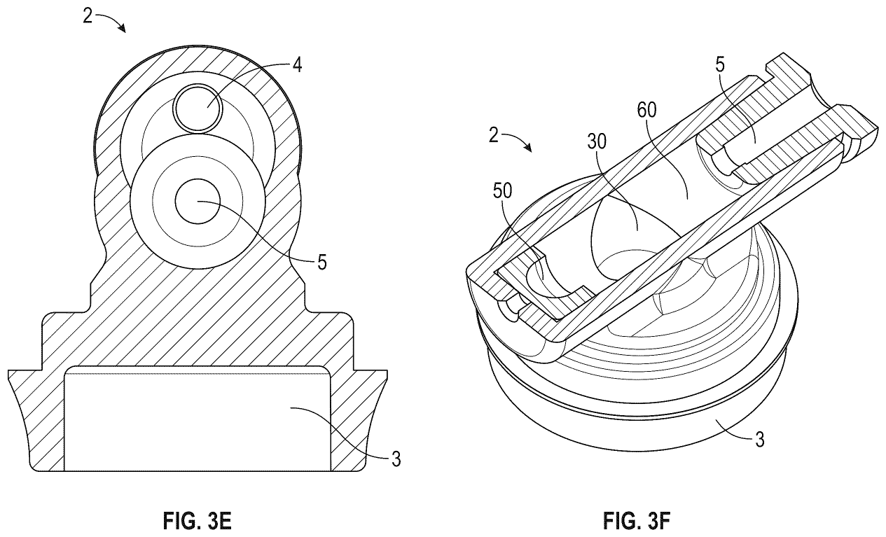

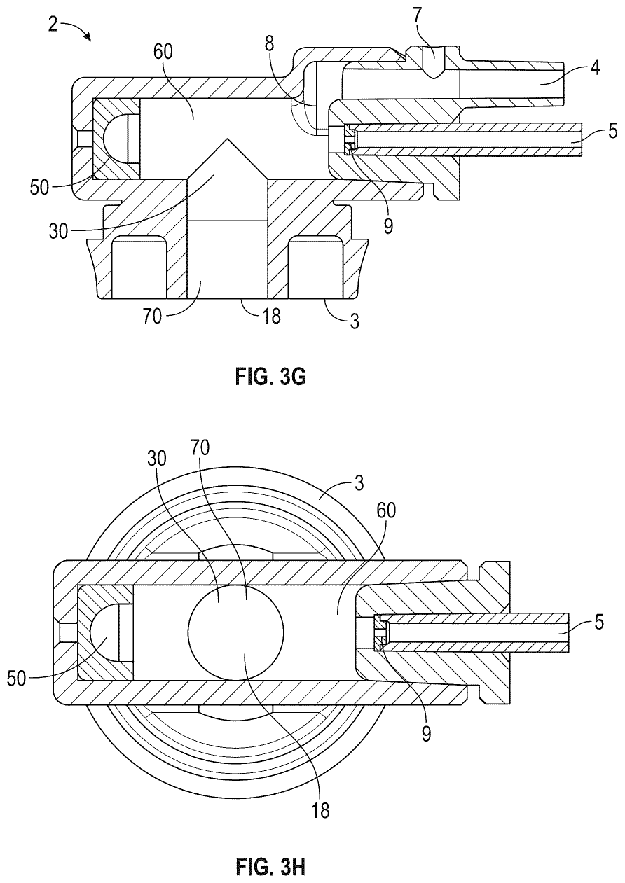

FIGS. 3A-3H illustrate another embodiment of a fluid platform 2 of a treatment instrument 1. The fluid platform 2 can be coupled to a distal portion of a handpiece 12 of the treatment instrument 1. In some embodiments, the fluid platform 2 can form part of a removable tip device 11 that can be removably connected to the handpiece 12. In other embodiments, the fluid platform 2 can be non-removably attached to the handpiece 12 or can be integrally formed with the handpiece 12. In still other embodiments, the fluid platform 2 may not couple to the handpiece 12 and may instead serve as a treatment cap that is adhered (or otherwise coupled or positioned) to the tooth without using a handpiece.

As shown in FIGS. 3A and 3D, a vent 7 can be provided through a portion of the fluid platform 2 to provide fluid communication between an evacuation line or outlet line 4 and ambient air. The vent 7 can serve to regulate pressure in the fluid platform 2 and can improve the safety and efficacy of the treatment instrument.

As shown in FIGS. 3B and 3D, an access port or opening 18 can be provided at a distal portion of the fluid platform 2 to provide fluid communication between a chamber 70 defined by the fluid platform 2 and the treatment region of the tooth 110. For example, in root canal cleaning procedures, a sealing cap 3 at the distal portion of the fluid platform 2 can be positioned against the tooth over an endodontic access opening 118 to provide fluid communication between the distal chamber 70 and the interior of the tooth (e.g., the pulp cavity and root canal(s)). In other embodiments, the sealing cap 3 can be positioned against the tooth 110 over the carious region at an exterior surface 119 of the tooth 110 to provide fluid communication between the distal chamber 70 and the carious region to be treated. In some alternative embodiments, a curable material can be provided on a sealing surface of the fluid platform 2. The curable material can be applied to the tooth and can cure to create a custom platform and seal that can be removable and reusable. In some embodiments, a conforming material can be provided on the sealing surface of the tooth. The conforming material may cure or harden to maintain the shape of the occlusal surface.

As described in further detail herein, pressure waves 23 and fluid motion 24 generated within the fluid platform 2 can propagate throughout the treatment region to clean and/or fill the treatment region.

The fluid platform 2 can include a proximal chamber 60. In some embodiments, the proximal chamber 60 and distal chamber 70 can together form a chamber 6 of the fluid platform 2. A transition opening 30 provided at a junction between the proximal chamber 60 and the distal chamber 70 can provide fluid communication between the proximal chamber 60 and the distal chamber 70. As shown, the access opening 18 can be disposed distal the transition opening 30, and the transition opening 30 can be disposed distal the nozzle 9.

A pressure wave generator 10 (which can serve as a fluid motion generator) can be arranged to generate pressure waves and/or rotational fluid motion in the proximal chamber 60 to cause pressure waves and/or rotational fluid motion to propagate to the treatment region (through the transition opening 30, through the distal chamber 70, and through the access opening 18). The pressure wave generator 10 can be disposed outside the tooth during a treatment procedure. The pressure wave generator 10 can comprise a liquid supply port that can deliver a liquid stream (such as a liquid jet) across the proximal chamber 60 to impinge upon an impingement surface (e.g., completely across the proximal chamber 60 to impinge upon an impingement surface opposite the pressure wave generator 10 or supply port) to generate pressure waves and fluid motion. For example, the pressure wave generator 10 can comprise a liquid jet device that includes an orifice or nozzle 9. Pressurized liquid can be transferred to the nozzle 9 along a pressurized fluid supply line or inlet line 5. The inlet line 5 can be connected to a fluid source in a console, for example, by way of one or more conduits 104. The nozzle 9 can have a diameter selected to form a high velocity, coherent, collimated liquid jet. The nozzle 9 can be positioned at a distal end of the inlet line 5. In various embodiments disclosed herein, the nozzle 9 can have an opening with a diameter in a range of 55 microns to 75 microns, in a range of 59 microns to 69 microns, in a range of 60 microns to 64 microns, or in a range of 61 microns to 63 microns. For example, in one embodiment, the nozzle 9 can have an opening with a diameter of approximately 62 microns, which has been found to generate liquid jets that are particularly effective at cleaning teeth. Although the illustrated embodiments are configured to form a liquid jet (e.g., a coherent, collimated jet), in other embodiments, the liquid stream may not comprise a jet but instead a liquid stream in which the momentum of the stream is generally parallel to the stream axis.

The nozzle 9 can be configured to direct a liquid stream comprising a liquid jet laterally through a laterally central region of the proximal chamber 60 along a jet axis X (also referred to as a stream axis) non-parallel to (e.g., substantially perpendicular to) a central axis Z extending through the distal chamber (e.g., passing through the approximate geometric center of the access port 18 and/or the transition opening 30). In some embodiments, the jet axis X can intersect the central axis Z. In various embodiments, the liquid stream (e.g., the jet) can intersect the central axis Z. In other embodiments, the jet axis X can be slightly offset from the central axis Z. In some embodiments, the liquid jet can generate fluid motion 24 (e.g., vortices, toroidal flow, turbulent flow) that can propagate throughout the treatment region (e.g., throughout a root canal, throughout a carious region on an external surface of the tooth, etc.) to interact with and remove unhealthy material. In some embodiments, the pressure wave generator 10 can generate broadband pressure waves through the fluid in the proximal chamber 60 and distal chamber 70 to clean the treatment region.

The nozzle 9 can form the coherent, collimated liquid jet 20. During operation, the proximal chamber 60 and distal chamber 70 can fill with the treatment liquid supplied by the liquid jet 20 (and/or additional inlets to the proximal chamber 60). The jet can enter the proximal chamber 60 and can interact with the liquid retained in the proximal chamber 60. In some embodiments, the interaction between the liquid jet 20 and the liquid in the proximal chamber 60 can create the pressure waves, which can propagate throughout the treatment region.

The fluid platform 2 can include an impingement member 50, which can be positioned such that the liquid jet 20 (e.g., located opposite the nozzle 9 along the jet axis X) impacts the impingement member 50 during operation of the pressure wave generator 10. The impingement member 50 can be sized, shaped (e.g., having one or more curved and/or angled surfaces), and/or otherwise configured such that, when the jet impinges on or impacts the impingement member 50, the movement of the jet is diverted or redirected back over the transition opening 30. For example, in some embodiments the impingement member 50 can be generally concave. In some embodiments, the impingement member 50 can be a curved surface in the shape of a hemispherical recess.

In some embodiments, fluid motion 24 may be affected by a location on the impingement member 50 at which the jet contacts the impingement member 50 and/or an angle at which the jet contacts the impingement member 50. In some embodiments, the impingement member 50 and/or nozzle 9 can be positioned so that the jet axis X is aligned with a center point of the impingement member 50 as shown in FIG. 3D. In other embodiments, as described in further detail with respect to FIGS. 11A-11J, the jet axis X can be offset from a center point of the impingement member 50 (e.g., superior or inferior to the center point of the impingement member 50). For example, in some embodiments, the jet axis X may be aligned with a superior section of the impingement member 50, so that the fluid from the fluid jet is biased to flow downward around the curved and/or angled surfaces of the impingement member 50 to cause more of the redirected fluid to flow below the center of the impingement member 50 and closer to the transition opening 30. In some embodiments, as explained above, the jet axis X can be disposed substantially perpendicular to the central axis Z. In other embodiments, the jet axis X can be angled relative to the central axis Z at an angle in a range of 45° to 135°, in a range of 60° to 120°, or in a range of 75° to 105°. In some embodiments, it may be desirable that a maximum amount of the redirected flow flows over the transition opening 30.

In some embodiments, the redirected fluid or jet can induce fluid motion 24 within the distal chamber 70 when flowing over the transition opening 30 after impingement on the impingement member 50. In some embodiments, the fluid motion induced in the distal chamber 70 when the redirected fluid or jet flows over the transition opening 30 can include turbulent flow including vortices, cyclonic flow, and/or toroidal flow. In some embodiments, the fluid motion 24 induced in the distal chamber 70 when the redirected fluid or jet flows over the transition opening 30 can be different at different times (e.g., toroidal flow at a first time and cyclonic flow at a second time), such that the flow profile in the distal chamber 70 can vary during the treatment procedure and/or be chaotic. In some embodiments, when the jet impinges on or impacts the impingement member 50, fluid motion 24 is created along the impingement member 50 (e.g., along the one or more curved or angled surfaces), along the interior surfaces of the proximal chamber 60, and/or within the fluid retained in the proximal chamber 60. Moreover, the movement of the jet and/or the liquid stream diverted by the impingement member 50 can induce fluid motion 24 in the proximal chamber 60. In some embodiments, an interaction of the fluid of the jet flowing towards the impingement member 50 and the fluid of the jet after redirection by the impingement member 50 can induce fluid motion 24, for example, small vortices, turbulent flow, and/or chaotic flow. In some embodiments, some of the fluid motion 24 within the proximal chamber 60 can propagate into the distal chamber 70 to cause turbulence within the distal chamber 70, for example, by inducing shear stresses in the fluid in the distal chamber 70.

The combination of the different types of fluid motion 24 that can be generated by propagation and redirection of the jet within the proximal chamber 60 can result in fluid motion 24 within the proximal chamber 60 and/or the distal chamber 70 that can be turbulent in nature and may rotate about multiple axes, which can increase the chaotic or turbulent nature of the flow and improve treatment efficacy. In some embodiments, the fluid motion 24 can propagate through the treatment region and can provide bulk fluid motion that flushes undesirable material (e.g., decayed organic matter) out of the treatment region. The combination of the fluid motion 24 and broadband generated pressure waves 23 can effectively remove undesirable materials of all shapes and sizes from large and small spaces, cracks, and crevices of the treatment region. In some embodiments, the fluid flow 24 can have sufficient momentum and structure to reach large and small spaces, cracks, and crevices of the treatment region. The fluid motion 24, which may be described as turbulent or unsteady, can include small eddies and may be non-repeating. Examples of fluid motion 24 that can occur within the fluid platform 2 are illustrated by arrows in FIG. 3D.

The combination of different types of fluid motion 24 can create unsteady flow such that, over the course of a treatment procedure, the fluid flow does not reach steady state. Some treatment instruments may induce fluid motion 24 in the treatment region that reaches a steady state after a time period. Steady flow can reduce treatment efficacy, for example, because the flow vectors of the treatment fluid do not change sufficiently so as to reach small untreated spaces that may be located along non-linear tubules or other spaces or cracks. Beneficially, the arrangement of the pressure wave generator 10, impingement member 50, the proximal chamber 60, and the distal chamber 70 can cooperate to generate non-steady flow during operation in a treatment procedure. Non-steady flow can create changing flow direction and/or changing flow vectors that increase the probability that, over the course of the treatment, the treatment fluid will reach remote regions that would otherwise be difficult or impossible to reach with steady state operational devices.

As shown in the embodiment of FIGS. 3A-3H, in certain embodiments, the impingement member 50 may be a separate piece that can be positioned within the proximal chamber 60. Alternatively, the impingement member 50 may be a curved or angled sidewall of the proximal chamber 60 (e.g., the impingement member 50 may be integrally or monolithically formed with the wall of the proximal chamber 60). For example, in some embodiments, the impingement member may be a sidewall 13 as described with respect to FIGS. 2A-K.

The fluid platform 2 can also include an evacuation or outlet line 4 to convey waste or effluent liquids to a waste reservoir, which may be located, for example, in a system console 102. A suction port 8 or fluid outlet can be exposed to the proximal chamber 60 along a wall of the proximal chamber 60 offset from the central axis Z. For example, as shown in FIG. 3D, the suction port 8 can be disposed along an upper wall of the proximal chamber 60 opposite the transition opening 30. A vacuum pump (not shown) can apply vacuum forces along the outlet line 4 to draw waste or effluent liquids 19 out of the proximal chamber 60 through the suction port 8, along the outlet line 4, and to the waste reservoir. In some embodiments, only one suction port 8 can be provided. In other embodiments, the fluid platform 2 can include a plurality (e.g., two) suction ports positioned laterally opposite one another. In some embodiments, more than two suction ports can be provided. In some embodiments, the drawing of fluid out of the proximal chamber 60 by the suction port 8 can affect the fluid motion 24 in the proximal chamber 60. For example, the action of the suction port 8 can withdraw at least some fluid from the liquid jet 20 that has passed back over the transition opening 30 after impingement on the impingement member 50. In some embodiments, this action of the suction port may prevent or reduce stagnation within the fluid in the proximal chamber 60 and/or may contribute to turbulent or chaotic fluid motion as described herein.

As shown in FIG. 3C, in some embodiments, the outlet line 4 and pressurized fluid inlet line 5 can be part of a separate manifold 80 that can couple to a main body 40 to form the fluid platform 2. The impingement member 50 may be pressed into the main body 40 or overmolded. The impingement member 50 may be metallic, ceramic, or formed of any other suitable material for receiving and redirecting the fluid jet.

FIGS. 3G and 3H depict example dimensions for the embodiment shown in FIGS. 3A-3F. As shown, the proximal chamber 60 and the distal chamber 70 can each be generally cylindrical in shape. A longitudinal axis of the cylindrical proximal chamber 60 (which in the illustrated embodiment may be coextensive or parallel with the jet axis X) can extend perpendicularly to a longitudinal axis of the cylindrical distal chamber 70 (which in the illustrated embodiment may be coextensive or parallel with the central axis Z). As shown in FIGS. 3A-3H, the proximal chamber 60 and distal chamber 70 have different geometries and/or volumes. In the illustrated embodiment, the impingement member 50 is disposed longitudinally beyond the transition opening 30 along the jet axis X such that the transition opening 30 is longitudinally between the impingement member 50 and the nozzle 9 along the jet axis X. In some embodiments, a jet length (i.e., a distance between the nozzle and an impingement point) can be between 1 mm and 20 mm, between 3 mm and 10 mm or any other suitable length. In some embodiments, a diameter of the proximal chamber 60 can be between 0.1 mm and 20 mm, between 1 mm and 10 mm, or any other suitable diameter. In some embodiments, a diameter of the distal chamber 70 can be between 0.5 mm and 10 mm, between 2 mm and 5 mm, or any other suitable diameter. In some embodiments, a height of the distal chamber 70 can be between 0 mm and 20 mm, between 0 mm and 6 mm or any other suitable height.

The proximal chamber 60 can accordingly have a first interior surface geometry 26a bounded by at least a wall 28a extending along upper, lower, and side surface(s) of the proximal chamber 60 and the impingement member 50. The distal chamber 70 can have a second interior surface geometry 26b bounded by at least a wall 28b extending along side surface(s) of the distal chamber 70. The first and second interior surface geometries 26a, 26b can be different as shown. For example, the first interior surface geometry 26a can comprise a curved surface (e.g., an approximately cylindrical surface) extending along the jet axis X from the nozzle 9 (or a location distal the nozzle 9) to the impingement surface of the impingement member 50. By contrast, the second interior surface geometry 26b can comprise a curved surface (e.g., an approximately cylindrical surface) extending distally along the central axis Z. The transition opening 30 can comprise a discontinuity that provides a non-uniform or abrupt flow transition between the proximal and distal chambers 60, 70. The discontinuity provided by the transition opening 30 and the differing interior surface geometries 26a, 26b can beneficially create unsteady flow of treatment fluid during operation of the treatment instrument in a treatment procedure. Non-uniform transitions can include asymmetric structures or irregularities in a transition region. The transition region can include the transition opening 30 and portions of the proximal chamber 60 and distal chamber 70 adjacent the transition opening 30. The asymmetric structures or irregularities may include one or more offsets, steps, recesses, or any other suitable structures.

In some embodiments, a ratio of a volume of the proximal chamber 60 to a volume of the distal chamber 70 is between 7:4 and 15:2. In some embodiments, a ratio of a volume of the proximal chamber 60 to a circumference of the transition opening 30 is between 1:150 and 1:20. In some embodiments, a ratio of a jet distance to a volume of the proximal chamber 60 is between 10:1 and 50:1. In some embodiments, a ratio of a jet distance to a jet height is between 2:1 and 13:2.

In some embodiments, the fluid platform 2 may include one or more additional fluid inlets, for example, for providing a filling material or filling material component. Additional fluid inlets may be positioned, for example, below the inlet 5 or below the impingement member 50. Additional details regarding embodiments with additional fluid inlets can be found throughout U.S. patent application Ser. No. 16/894,667, the entire contents of which are incorporated by reference herein in its entirety and for all purposes.

In the embodiment of FIGS. 3A-3H, the jet impinges on the impingement member 50, and the redirected flow can contribute to the fluid motion 24 in the distal chamber 70 and the treatment region. In other embodiments, there may be no impingement member 50 and instead the liquid jet may pass uninterrupted into a channel or tube at a location opposite the nozzle 9. In such embodiments, the liquid from the liquid jet may be conveyed away from the fluid platform 2 to a waste container or may be recirculated in a closed circuit to be reused. In such embodiments, therefore, the jet may not be redirected back proximally over the transition opening 30. The fluid motion in the distal chamber 70 and the treatment region can be induced by, e.g., at least the liquid jet being directed over the transition opening 30 and the distal chamber 70.

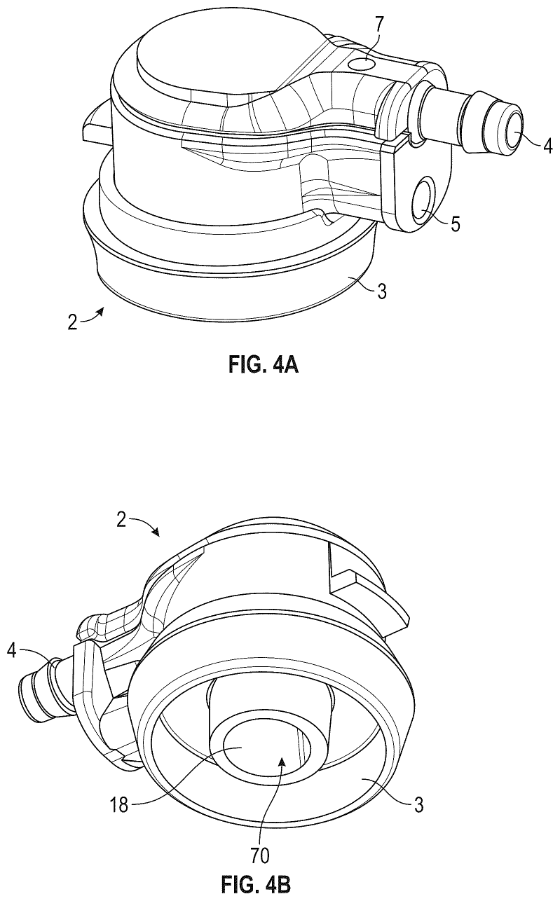

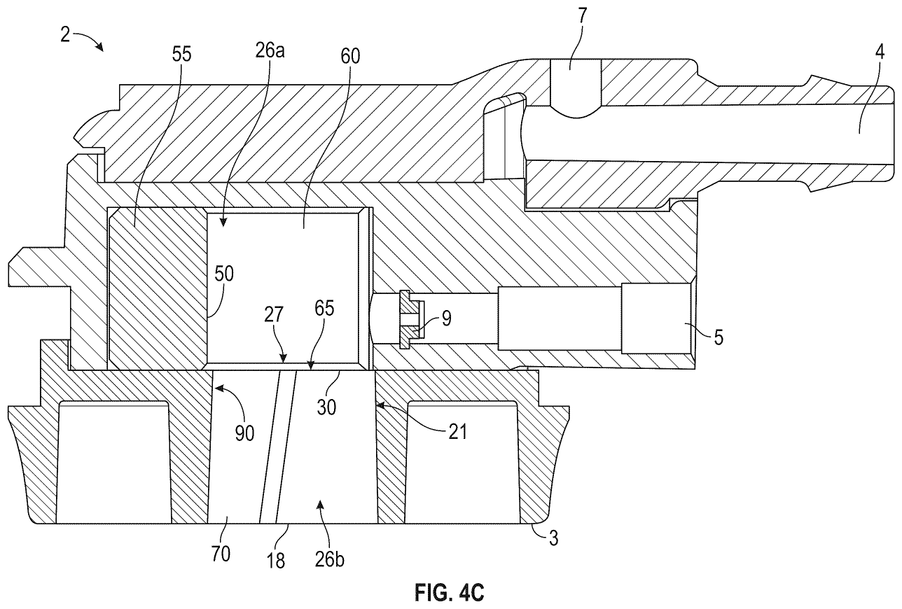

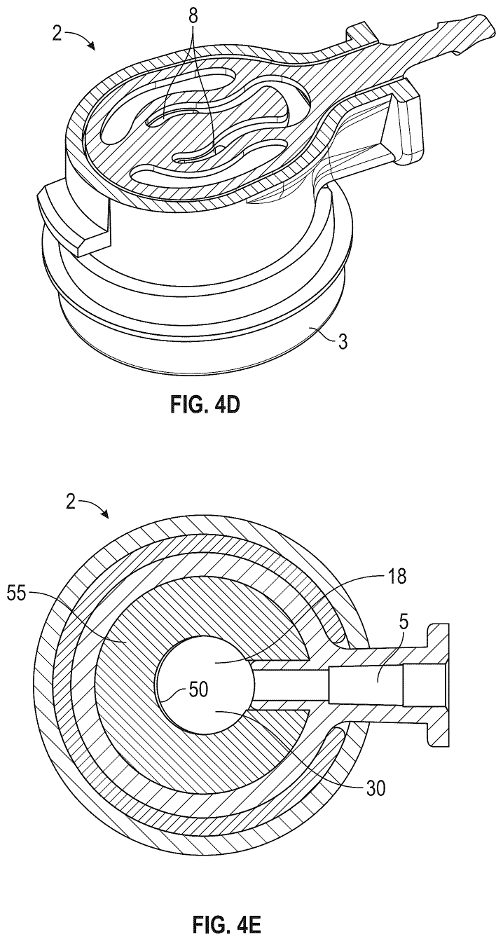

FIGS. 4A-4E depict another embodiment of a fluid platform 2. Unless otherwise noted, components of FIGS. 4A-4E may be generally similar to or the same as like-numbered components of FIGS. 3A-3H. In the embodiment of FIGS. 4A-4E, the impingement member 50 comprises a portion of inner wall of an impingement ring 55 positioned within the proximal chamber 60. The impingement ring 55 can be positioned within a housing of the fluid platform 2 and at least partially form the boundaries of the proximal chamber 60. The impingement ring 55 can extend around an interior section of the fluid platform 2 proximal to the distal chamber 70 and can have an opening configured to align with the fluid inlet line 5 and outlet line 4.

The impingement ring 55 can be seated on a surface 65 above the distal chamber 70. The surface 65 can define the transition opening 30. The impingement ring 55 can be positioned (e.g., seated on the surface 65) so as to create a non-uniform transition between the proximal chamber 60 and the distal chamber 70. For example, as shown in FIG. 4C, at least a portion of the impingement ring SS can be recessed relative to the transition opening 30 (e.g., by 0.005 in) to form a recess 90 and/or at least a portion of the impingement ring 55 can extend over the transition opening 30 (e.g., by 0.005 in) to form a ledge 21. In some embodiments, at least a portion 27 of the impingement ring 55 can also align with the transition opening 30. Without being limited by theory, it is believed that such a non-uniform transition or discontinuity can contribute to turbulent or chaotic fluid motion in the distal chamber 70 in an unsteady manner. Further, as explained herein, the non-uniform transition and different interior surface geometries 26a, 26b can enable operation in a non-steady state manner.

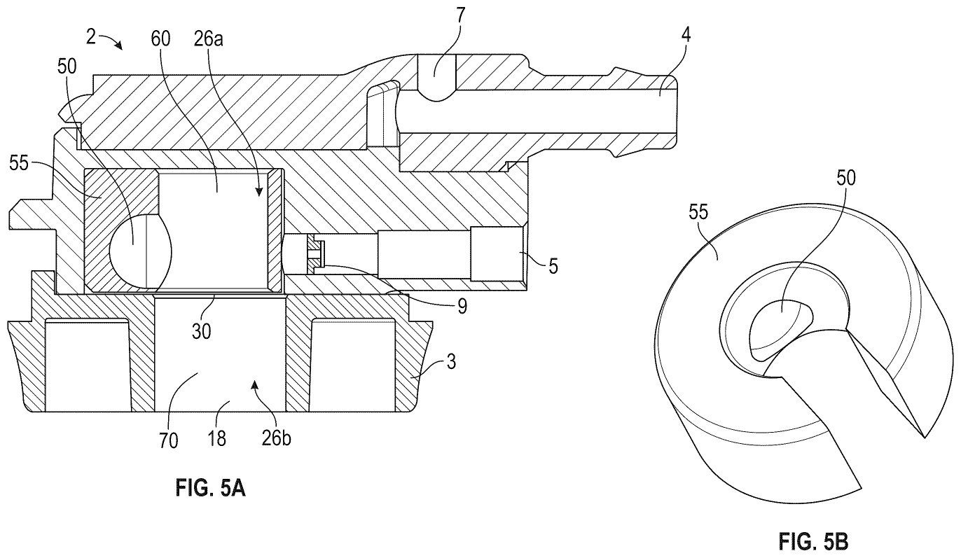

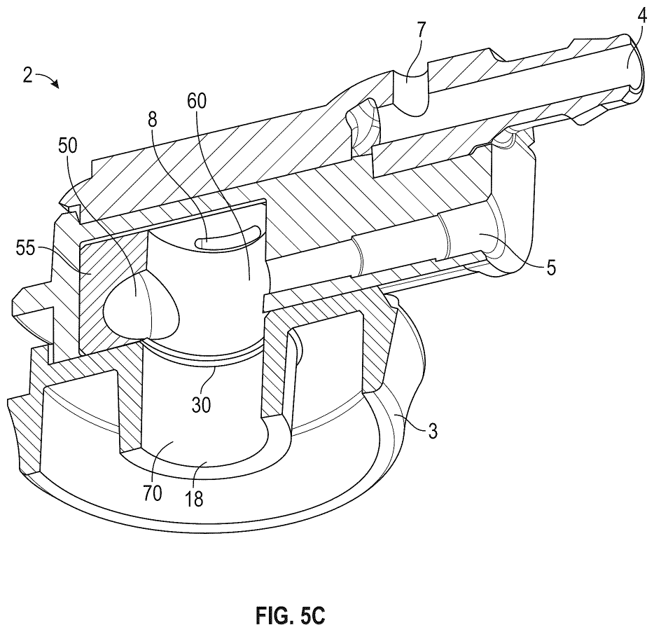

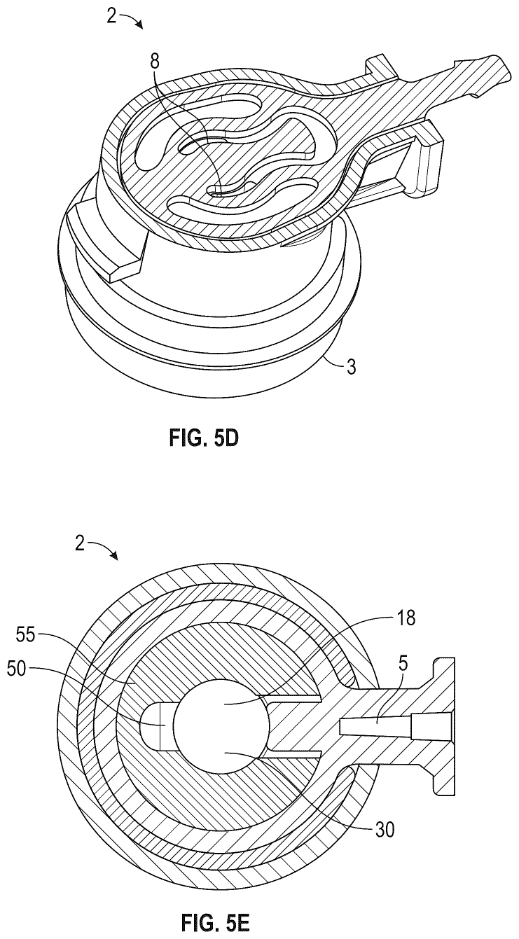

FIGS. 5A-5E depict an alternative embodiment of a fluid platform 2. In the embodiment of FIGS. 5A-E, the impingement member 50 can be in the form of a divot within the impingement ring 55. The divot 50 can be machined into the wall of the impingement ring 55. In some embodiments, divot 50 can have generally the same shape as the impingement member 50 of FIGS. 3A-3H.

In some embodiments, the impingement ring 55 of FIGS. 5A-5E can be positioned to create a non-uniform transition between the proximal chamber 60 and the distal chamber 70, for example, as described with respect to the embodiment of FIGS. 4A-4E. In other embodiments, the inner circumference of the distal end of the impingement ring 55 can align with the transition opening 30. Further, as explained above, the interior surface geometries 26a, 26b of the proximal and distal chambers 60, 70 may differ. The non-uniform transition and/or differing surface geometries 26a, 26b can beneficially generate unsteady flow of treatment fluid during a treatment procedure, as explained above.

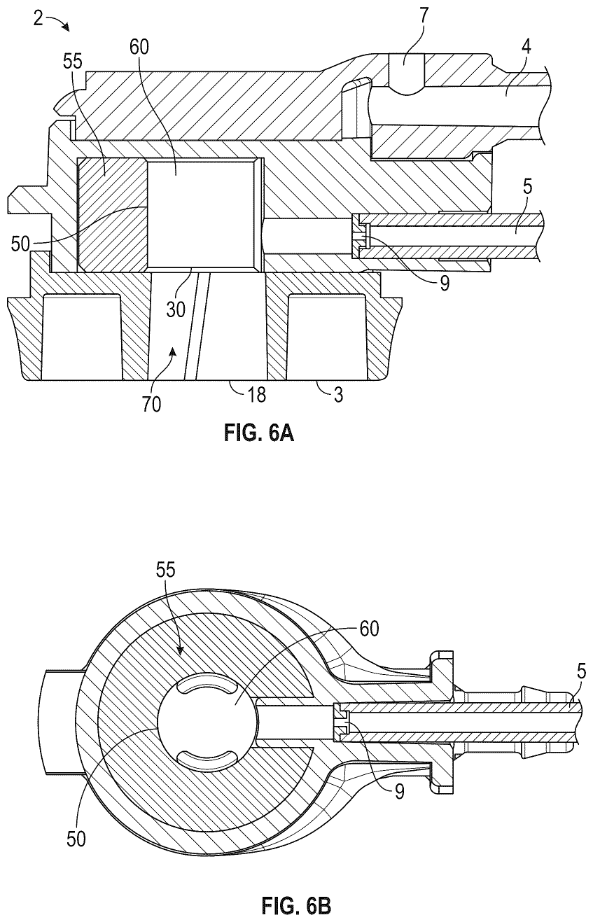

FIGS. 6A and 6B show example dimensions of an embodiment of a fluid platform 2 that may be generally the same or similar to the dimensions of the embodiments shown in FIGS. 4A-4E and 5A-5E. As shown, the proximal chamber 60 and the distal chamber 70 can each be generally cylindrical in shape. A longitudinal axis of the cylindrical proximal chamber 60 can extend generally in parallel to a longitudinal axis of the cylindrical distal chamber 70 or may be the same axis. In some embodiments, a jet length (i.e., a distance between the nozzle and an impingement point) can be between 1 mm and 20 mm, between 3 mm and 10 mm or any other suitable length. In some embodiments, a diameter of the proximal chamber 60 can be between 0.1 mm and 20 mm, between 1 mm and 10 mm, or any other suitable diameter. In some embodiments, a diameter of the distal chamber 70 can be between 0.5 mm and 10 mm, between 2 mm and 5 mm, or any other suitable diameter. In some embodiments, a height of the distal chamber 70 can be between 0 mm and 20 mm, between 0 mm and 6 mm or any other suitable height.

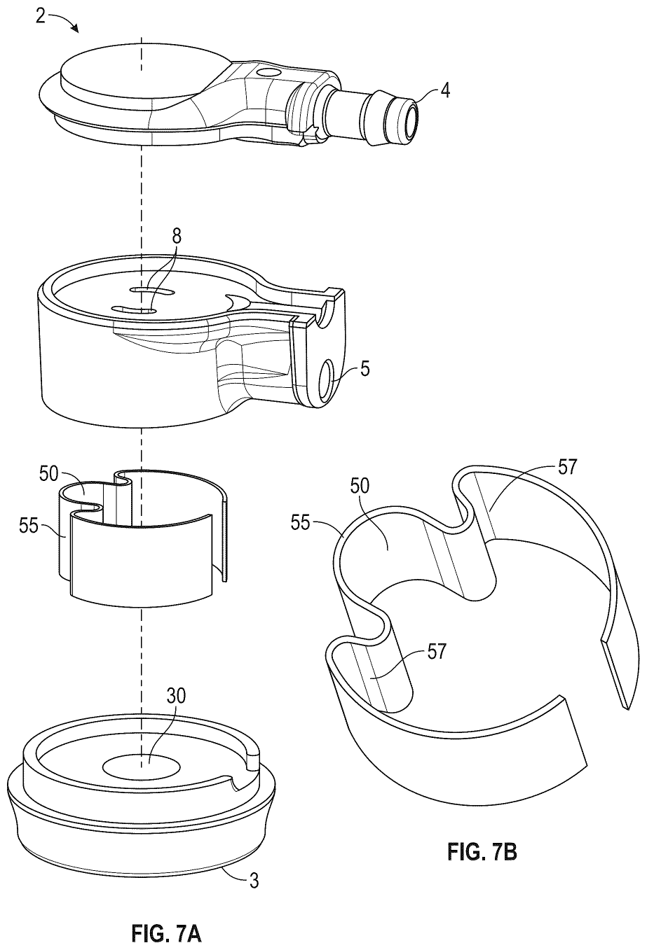

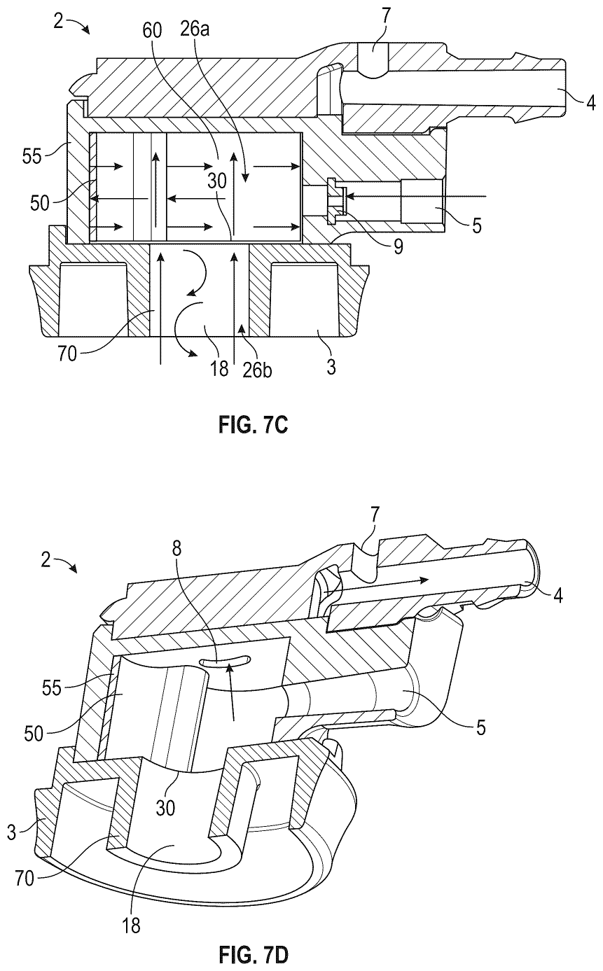

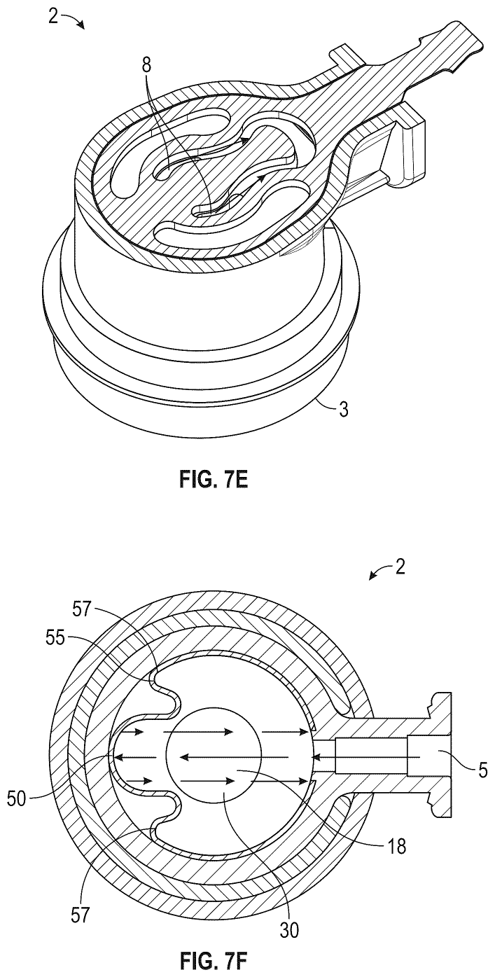

FIGS. 7A-7E depict another embodiment of a fluid platform 2. In the embodiment of FIGS. 7A-7E, the impingement ring 55 has a non-circular cross-section. The impingement member 50 is in the form of a curved impingement surface having sidewall sections that extend back towards the inlet line 5 so as to redirect fluid from the jet flowing along the sidewall sections towards the transition opening 30. The shape and size of sidewall sections of the impingement member 50 can increase the amount of fluid redirected over the transition opening 30 after impingement in comparison to impingement rings 55 having circular cross-sections (e.g., by directing fluid flowing along the sidewalls from the impingement surface towards the transition opening 30 instead of around the circumference inner surface of a circular impingement ring).

FIGS. 7C-7E include arrows showing examples of fluid motion within the proximal and distal chambers 60 and 70. The arrows in FIGS. 7D and 7E show the flow of fluid through the suction ports 8 and the outlet line 4.

As shown in FIGS. 7A-7E, the impingement ring 55 can include two additional recessed regions 57 formed by the curvature of the sidewall of the impingement ring adjacent the impingement member 50. In some embodiments, the additional recessed regions may provide additional vortices or turbulent fluid motion when interacting with other fluid motion in the proximal chamber 60. In some embodiments, the sections of the sidewall of the impingement ring 55 separating the impingement member 50 and the recessed regions 57 can act as flow disruptors. In some embodiments, the shape of the impingement ring 55 of FIGS. 7A-7E can promote sound propagation. As explained above, the impingement ring 55 of FIGS. 7A-7E can be positioned to create a non-uniform transition between the proximal chamber 60 and the distal chamber 70. Further, as explained above, the interior surface geometries 26a, 26b of the proximal and distal chambers 60, 70 may differ. The non-uniform transition and/or differing surface geometries 26a, 26b can beneficially generate unsteady flow of treatment fluid during a treatment procedure, as explained above.

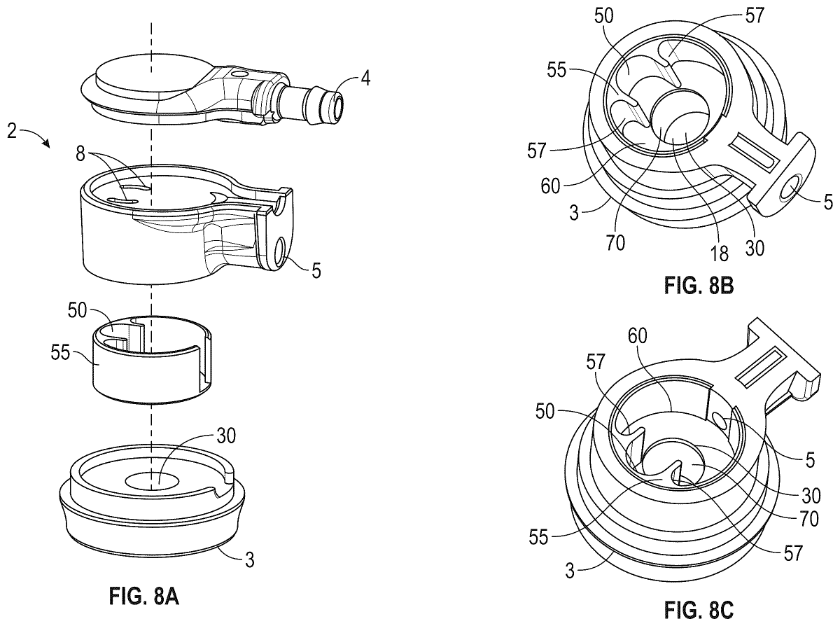

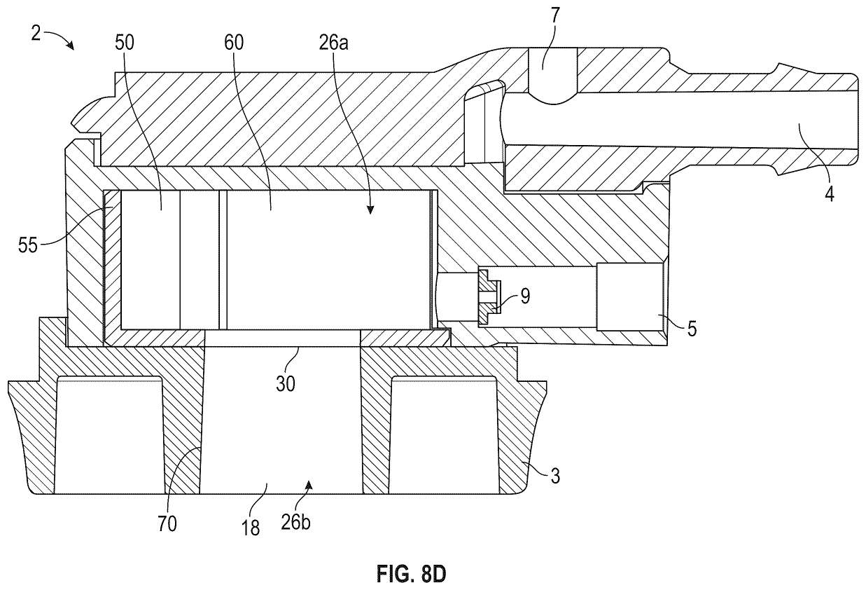

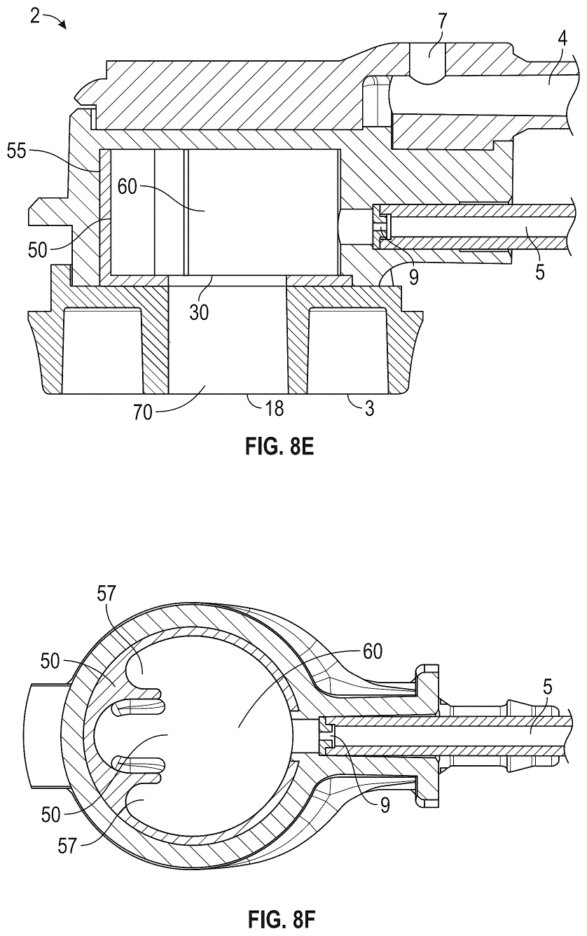

FIGS. 8A-8F depict an alternative embodiment of a fluid platform 2. Similar to the embodiment of FIGS. 7A-7E, in the embodiment of FIGS. 8A-8F, the inner walls of the impingement ring 55 can have a non-circular cross-section. The impingement member 50 is in the form of a curved impingement surface having sidewall sections that extend back towards the inlet line 5 so as to redirect fluid from the jet flowing along the sidewall sections towards the transition opening 30. The shape and size of sidewall sections of the impingement member 50 can increase the amount of fluid redirected over the transition opening 30 after impingement in comparison to impingement rings 55 having circular cross-sections (e.g., by directing fluid flowing along the sidewalls from the impingement surface towards the transition opening 30 instead of around the circumferential inner surface of a circular impingement ring).

As shown in FIGS. 8A-8F, the impingement ring 55 can include two additional recessed regions 57 formed by the curvature of the sidewall of the impingement ring adjacent the impingement member 50. In some embodiments, the additional recessed regions 57 may provide additional vortices or turbulent fluid motion when interacting with other fluid motion 24 in the proximal chamber 60. In some embodiments, the sections of the sidewall of the impingement ring 55 separating the impingement member 50 and the recessed regions 57 can act as flow disruptors. In some embodiments, the shape of the impingement ring 55 of FIGS. 8A-8F can promote sound propagation. As explained above, the impingement ring 55 of FIGS. 8A-8F can be positioned to create a non-uniform transition between the proximal chamber 60 and the distal chamber 70. Further, as explained above, the interior surface geometries 26a, 26b of the proximal and distal chambers 60, 70 may differ. The non-uniform transition and/or differing surface geometries 26a, 26b can beneficially generate unsteady flow of treatment fluid during a treatment procedure, as explained above.

FIGS. 8E and 8F show example dimensions the fluid platform 2 as shown in FIGS. 8A-8D. In some embodiments, a longitudinal axis of the proximal chamber 60 can be generally in parallel to a longitudinal axis of the distal or may be the same axis. The dimensions of the embodiment shown in FIGS. 7A-7E may be generally the same or similar. In some embodiments, a jet length (i.e., a distance between the nozzle and an impingement point) can be between 1 mm and 20 mm, between 3 mm and 10 mm or any other suitable length. In some embodiments, a diameter of the proximal chamber 60 can be between 0.1 mm and 20 mm, between 1 mm and 10 mm, or any other suitable diameter. In some embodiments, a diameter of the distal chamber 70 can be between 0.5 mm and 10 mm, between 2 mm and 5 mm, or any other suitable diameter. In some embodiments, a height of the distal chamber 70 can be between 0 mm and 20 mm, between 0 mm and 6 mm or any other suitable height.

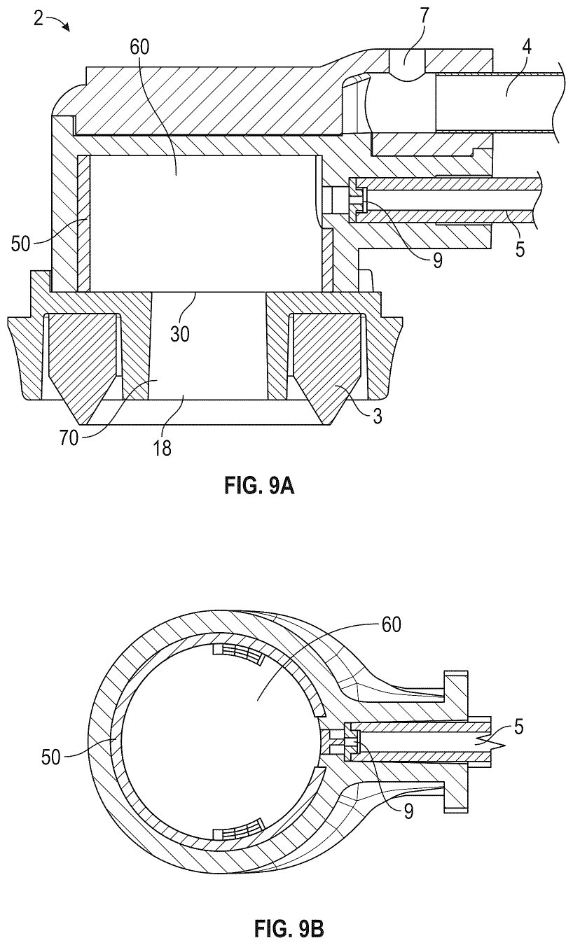

FIGS. 9A and 9B depicts an alternative embodiment of a fluid platform 2. In the embodiment of FIGS. 9A-9B, the impingement member 50 is a portion of a generally cylindrical inner wall of the proximal chamber 60. The inner wall of the proximal chamber 60 can be formed by an impingement ring 55. In some embodiments, the inner wall of the proximal chamber 60 can be formed by the fluid platform 2. FIGS. 9A and 9B show example dimensions of the fluid platform 2. As shown, the proximal chamber 60 and the distal chamber 70 can each be generally cylindrical in shape. In some embodiments, a jet length (i.e., a distance between the nozzle and an impingement point) can be between 1 mm and 20 mm, between 3 mm and 10 mm or any other suitable length. In some embodiments, a diameter of the proximal chamber 60 can be between 0.1 mm and 20 mm, between 1 mm and 10 mm, or any other suitable diameter. In some embodiments, a diameter of the distal chamber 70 can be between 0.5 mm and 10 mm, between 2 mm and 5 mm, or any other suitable diameter. In some embodiments, a height of the distal chamber 70 can be between 0 mm and 20 mm, between 0 mm and 6 mm or any other suitable height.

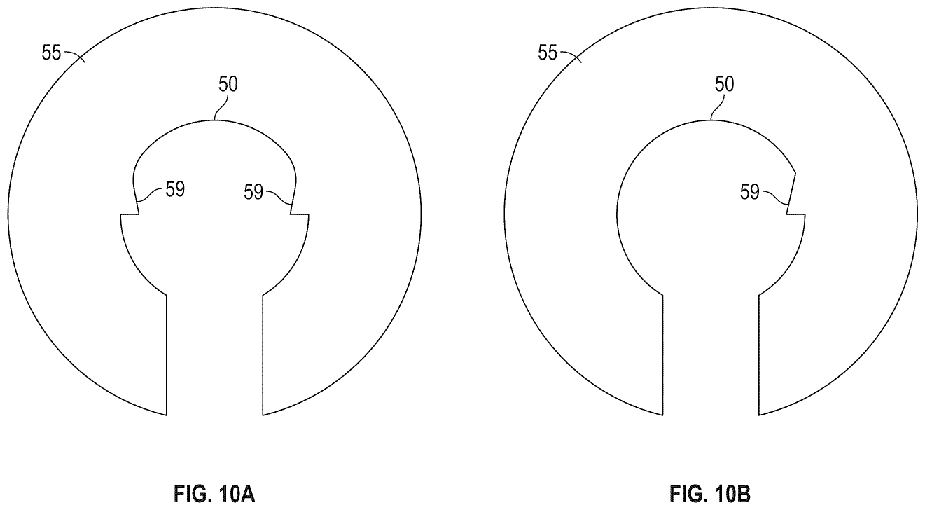

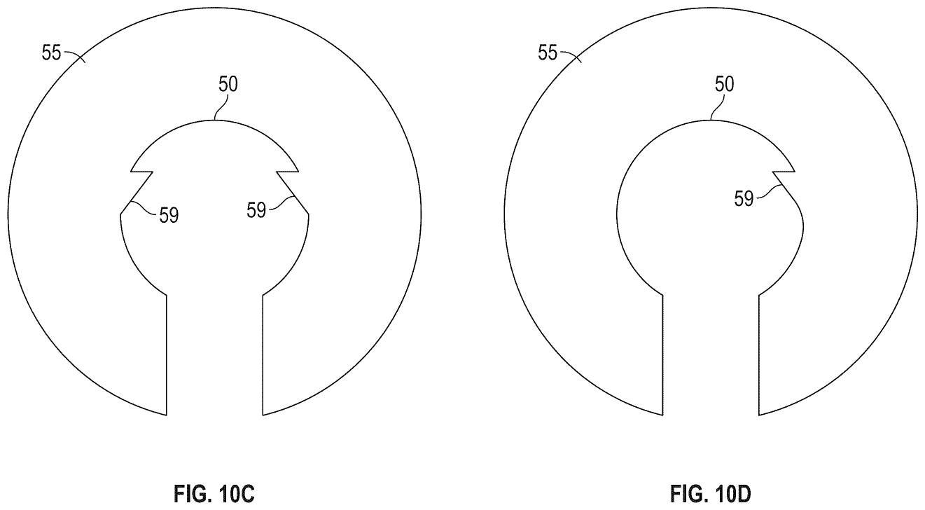

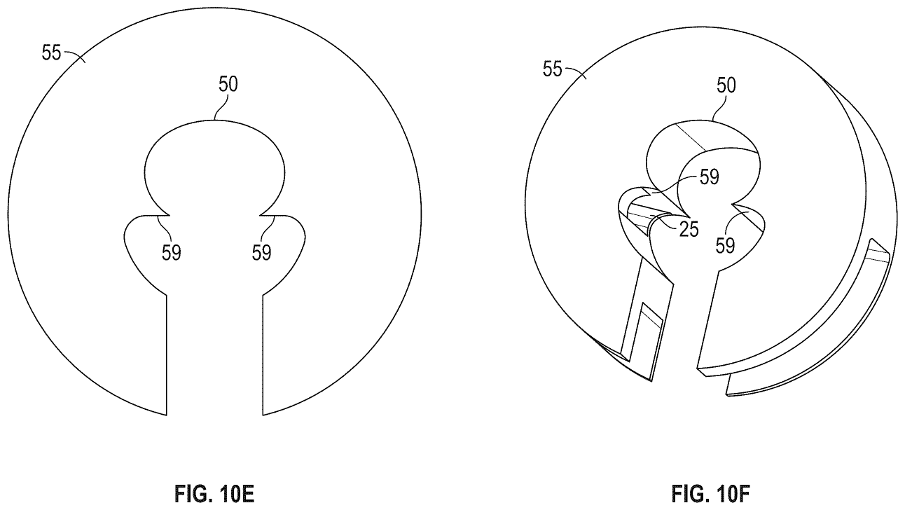

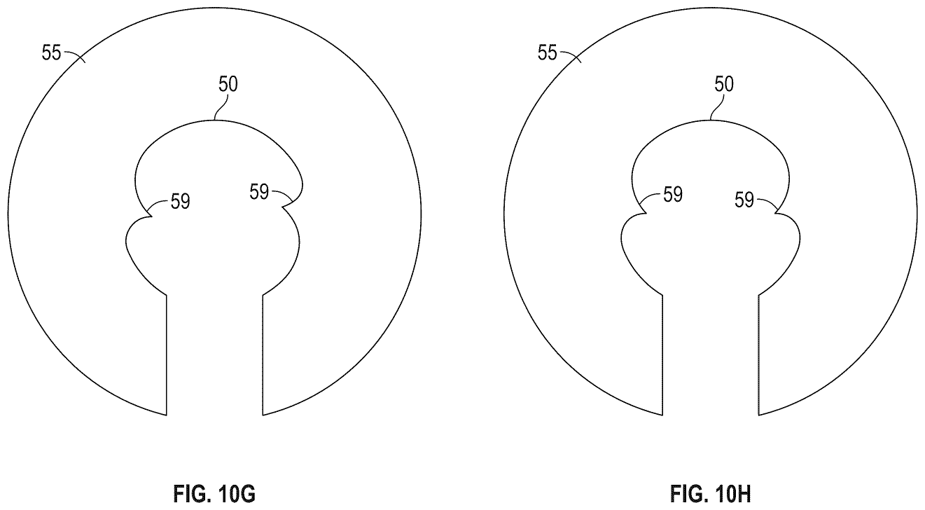

Additional examples of impingement rings 55 are shown in FIGS. 10A-10J. In some embodiments, the impingement rings can include one or more flow disruptors 59 that can disrupt fluid flow along the inner surface of the impingement ring 55 to generate fluid motion 24. The flow disruptors 59 may be in the form of pointed or curved protrusions extending inwardly from the inner surface of the impingement ring 55, for example, as shown in FIGS. 10A-10H. In some embodiments, the flow disruptors may be in the form of recesses formed in the inner surface of the impingement ring 55, for example, as shown in FIGS. 101 and 10J. In some embodiments, such as for example, in FIGS. 10A, 10C, 10F, and 10H-10J, the flow disruptors 59 may be symmetrical about a plane extending through the center of the impingement surface. In other embodiments, such as for example, in FIGS. 10B, 10D, and 10G, the disruptors 59 may be asymmetrical. The embodiment shown in FIG. 10I may cause spray in a plurality of different directions. In embodiments in which the impingement member 50 is a portion of an inner wall of the proximal chamber 60, flow disruptors 59 may extend from the inner wall of the proximal chamber 60.

As shown in FIG. 10F, in some embodiments, the impingement ring 55 may include a port 25 (such as a side port) which can be used to introduce additional fluids into proximal chamber 60, such as, for example, a filling material or a component of a filling material.

In some embodiments, the impingement ring may include an at least partially hollow interior that can form a guide path for the fluid jet instead of an impingement surface. The fluid jet can flow through the interior of the impingement ring 55 to another location within the proximal chamber 60 instead of impinging on the impingement surface.

In the embodiments shown in FIGS. 3A-10H, the impingement member 50 is positioned on an opposite side of the proximal chamber 60 from the fluid inlet 5 beyond the transition opening 30. In some embodiments, an impingement member 50 may be positioned over the transition opening 30. In some embodiments, an impingement member 50 may split the jet to cause the jet to flow in multiple directions above the transition opening 30.

FIGS. 11A-11J depict another embodiment of a fluid platform 2. The fluid platform 2 can be coupled to a distal portion of a handpiece 12 of a treatment instrument 1. In some embodiments, the fluid platform 2 can form part of a removable tip device that can be removably connected to the handpiece 12. In other embodiments, the fluid platform 2 can be non-removably attached to the handpiece 12 or can be integrally formed with a handpiece 12. In still other embodiments, the fluid platform 2 may not couple to a handpiece 12 and may instead serve as a treatment cap that is adhered (or otherwise coupled or positioned) to the tooth without using a handpiece. Unless otherwise noted, components of FIGS. 11A-11J may be generally similar to or the same as like-numbered components of FIGS. 2D-2K, FIGS. 3A-3H, FIGS. 4A-4E, FIGS. 5A-5E, FIGS. 6A-6B, FIGS. 7A-7F, FIGS. 8A-8F, and FIGS. 9A-9B.

As shown in FIG. 11A, a vent 7 can be provided through a portion of the fluid platform 2 to provide fluid communication between the evacuation line or outlet line 4 and ambient air. The vent 7 can serve to regulate pressure in the fluid platform 2 and can improve the safety and efficacy of the treatment instrument.

In some embodiments, the access port or opening 18 can be provided at a distal portion of the fluid platform 2 to provide fluid communication between a distal chamber 70 of the fluid platform 2 and the treatment region of the tooth 110. For example, in root canal cleaning procedures, a sealing cap 3 at the distal portion of the fluid platform 2 can be positioned against the tooth over an endodontic access opening to provide fluid communication between the distal chamber 70 and the interior of the tooth (e.g., the pulp cavity and root canal(s)). In other embodiments, the sealing cap 3 can be positioned against the tooth 110 over the carious region at an exterior surface of the tooth 110 to provide fluid communication between the distal chamber 70 and the carious region to be treated. In some alternative embodiments, a curable material can be provided on a sealing surface of the fluid platform 2. The curable material can be applied to the tooth and can cure to create a custom platform and seal. In some embodiments, the custom platform can be removable and reusable. In some embodiments, a conforming material can be provided on the sealing surface of the tooth. The conforming material may cure or harden to maintain the shape of the occlusal surface.

As described in further detail herein, pressure waves 23 and fluid motion 24 generated with in the fluid platform 2 can propagate throughout the treatment region to clean and/or fill the treatment region.

The fluid platform 2 may include a proximal chamber 60. In some embodiments, the proximal chamber 60 and distal chamber 70 can together form a chamber 6 of the fluid platform 2. A transition opening 30 provided at a junction between the proximal chamber 60 and the distal chamber 70 can provide fluid communication between the proximal chamber 60 and the distal chamber 70. As shown, the access opening 18 can be disposed distal the transition opening 30, and the transition opening 30 can be disposed distal the nozzle 9.

A pressure wave generator 10 (which can serve as a fluid motion generator) can be arranged to generate pressure waves and/or rotational fluid motion in the proximal chamber 60 to cause pressure waves and/or rotational fluid motion to propagate to the treatment region (through the transition opening 30, through the distal chamber 70, and through the access opening 18). The pressure wave generator 10 can be disposed outside the tooth during a treatment procedure. The pressure wave generator 10 can comprise a liquid supply port that can deliver a liquid stream (such as a liquid jet) across the proximal chamber 60 to impinge upon an impingement surface 53 (e.g., completely across the proximal chamber 60 to impinge upon an impingement surface 53 opposite the pressure wave generator 10 or supply port) to generate pressure waves and fluid motion. For example, the pressure wave generator 10 can comprise a liquid jet device that includes an orifice or nozzle 9. Pressurized liquid can be transferred to the nozzle 9 along a pressurized fluid supply line or inlet line 5. The inlet line 5 can be connected to a fluid source in a console, for example, by way of one or more conduits 104. The nozzle 9 can have a diameter selected to form a high velocity, coherent, collimated liquid jet. The nozzle 9 can be positioned at a distal end of the inlet line 5. In various embodiments disclosed herein, the nozzle 9 can have an opening with a diameter in a range of 55 microns to 75 microns, in a range of 54 microns to 64 microns, in a range of 57 microns to 61 microns, in a range of 58 microns to 60 microns, in a range of 59 microns to 69 microns, in a range of 60 microns to 64 microns, in a range of 61 microns to 63 microns, in a range of 63 microns to 73 microns, in a range of 66 microns and 70 microns, or in a range of 67 microns to 69 microns. For example, in one embodiment, the nozzle 9 can have an opening with a diameter of approximately 62 microns, which has been found to generate liquid jets that are particularly effective at cleaning teeth. In some embodiments, the nozzle can have an opening with a diameter of approximately 59 microns, which has been found to generate liquid jets that are particularly effective at cleaning teeth (e.g., premolar teeth). In some embodiments, the nozzle can have an opening with a diameter of approximately 68 microns, which has been found to generate liquid jets that are particularly effective at cleaning teeth (e.g., molar teeth and/or premolar teeth). Although the illustrated embodiments are configured to form a liquid jet (e.g., a coherent, collimated jet), in other embodiments, the liquid stream may not comprise a jet but instead a liquid stream in which the momentum of the stream is generally parallel to the stream axis.

FIG. 11F includes three-dimensional coordinate axes indicating superior (S), inferior (I), anterior (A), posterior (P), left (L), and right (R) directions. The superior direction corresponds to the proximal direction as described herein. The inferior direction corresponds to the distal direction as described herein. The super-inferior axis may be referred to as a vertical axis. As shown in FIG. 11F, the right direction R is generally pointing into the page and the left direction L is generally pointing out of the page. These directions are provided for reference only to provide examples of relative positions of components and directions of fluid motion within the fluid platform 2 and may not reflect the particular anatomical positions of components or directions of fluid motion when the fluid platform is in use.

The nozzle 9 can be configured to direct a liquid stream comprising a liquid jet 20 generally laterally (e.g., generally in the anterior direction) through a laterally central region of the proximal chamber 60 along a jet axis X′ (also referred to as a stream axis) non-parallel to (e.g., substantially perpendicular to or at an angle α to) a central axis Z extending through the distal chamber (e.g., passing through the approximate geometric center of the access port 18 and/or the transition opening 30). The central axis Z can be generally parallel with the superior-inferior axis as shown in FIG. 11F.

The nozzle 9 can be positioned at different locations vertically (along the superior-inferior axis) within the proximal chamber 60 and/or at different locations horizontally (along the left-right axis) within the proximal chamber 60. The jet axis X′ can include components in the anterior direction and, in some embodiments, in one or more of a superior/inferior direction or a left/right direction.

In some embodiments, the jet axis X′ can be positioned at an angle R relative to an axis X″ perpendicular to the central axis Z (e.g., the jet axis X′ can be directed both anteriorly and superiorly or inferiorly). In some embodiments, the axis X″ can be generally parallel to the anterior-posterior axis as shown in FIG. 11F. In some embodiments, the jet axis X′ can intersect the central axis Z. In various embodiments, the liquid stream (e.g., the liquid jet 20) can intersect the central axis Z. In other embodiments, the jet axis X′ and thus the liquid jet 20 can be offset from the central axis Z. For example, the jet axis X′ can be directed both anteriorly and horizontally left or right or the nozzle 9 can be positioned horizontally within the proximal chamber 60 such that a jet 20 directed solely in the anterior direction is offset to the left or right of the central axis Z (for example, to direct the jet 20 at a contact point 72 as described below).

In some embodiments, the liquid jet can generate fluid motion 24 (e.g., vortices, toroidal flow, turbulent flow) that can propagate throughout the treatment region (e.g., throughout a root canal, throughout a carious region on an external surface of the tooth, etc.) to interact with and remove unhealthy material. The fluid motion generator 10 can also act as a pressure wave generator to generate broadband pressure waves through the fluid in the proximal chamber 60 and distal chamber 70 to clean the treatment region.

The nozzle 9 can form the coherent, collimated liquid jet 20. During operation, the proximal chamber 60 and distal chamber 70 can fill with the treatment liquid supplied by the liquid jet 20 (and/or additional inlets to the proximal chamber 60). The jet can enter the proximal chamber 60 and can interact with the liquid retained in the proximal chamber 60. In some embodiments, the interaction between the liquid jet 20 and the liquid in the proximal chamber 60 can create the pressure waves, which can propagate throughout the treatment region.

The fluid platform 2 can include an impingement member 50, which can be positioned such that the liquid jet 20 (e.g., located opposite the nozzle 9 along the jet axis X′) impacts the impingement member 50 during operation of the pressure wave generator 10 (e.g., impacts an impingement surface 53 of the impingement member 50). The impingement member 50 can be sized, shaped (e.g., having one or more curved and/or angled surfaces, such as impingement surface 53), and/or otherwise configured such that, when the jet impinges on or impacts the impingement member 50, the movement of the jet is diverted or redirected back over the transition opening 30. For example, in some embodiments the impingement member 50 and/or impingement surface 53 can be generally concave. In some embodiments, the impingement surface 53 can be a curved surface in the shape of a hemispherical recess. Furthermore, in some embodiments, the fluid jet 20 may redirect off the impingement member 50 (e.g., redirect off the impingement surface 53) tangential to the hemispherical recess of the impingement member 50.

In some embodiments, the impingement member 50 may be disposed within the fluid platform 2 in a relatively vertical position, that is, with its posterior facing edge aligned substantially parallel with the central axis Z. In some embodiments, in the vertical position, a central axis X′″ of the impingement surface 53 may be generally perpendicular to the central axis Z. The central axis X′″ may also be a central axis of the impingement member 50. In some embodiments, as shown in FIG. 11D, the impingement member 50 may be disposed within the fluid platform 2 at an angle (e.g., at a downward angle towards transition opening 30), such that its posterior facing edge is not parallel with central axis Z and such that the central axis X′″ is non-parallel and non-perpendicular to the central axis Z (e.g., the axis X′ can have components in an inferior direction or a superior direction). As shown in FIG. 11D, the posterior facing edge of impingement member 50 may be substantially perpendicular to the jet axis X′, which itself is at a non-parallel angle α relative to central axis Z. In some embodiments, the impingement member 50 may be disposed within the fluid platform 2 at an angle such that the central axis X′″ of the impingement surface 53 is offset horizontally (e.g., to the left or to the right) from and does not intersect the central axis Z. For example, the posterior facing edge of the impingement member 50 can be non-parallel to a normal vector of a plane formed by axis Z and axis X′ when the two axes intersect.

In some embodiments, the form of the redirected fluid from the liquid jet 20 after impingement on the impingement member may be affected by a location on the impingement surface 53 at which the jet 20 contacts the impingement surface 53 and/or an angle at which the jet 20 contacts the impingement surface 53. For example, in some embodiments, the liquid jet 20 may be redirected as a spray. In other embodiments, for example, as shown in FIG. 11D, the liquid jet 20 may be redirected as a stream 29 in the form of a second liquid jet. In some embodiments, the liquid jet 20 may be redirected partially as a spray and partially as a redirected stream 29 in the form of a liquid jet. As used herein, “in the form of a liquid jet” means that the redirected fluid has characteristics of a liquid jet. For example, the redirected stream 29 may have characteristics similar to those of a stream formed from a small opening, such as a nozzle. The redirected stream 29 in the form of the liquid jet may maintain jet like qualities of flow after redirection from the impingement member 50. In some embodiments, the redirected jet-like stream 29 can have a generally circular cross-sectional profile. In some embodiments, the liquid jet 20 may be redirected as a sheet of liquid (e.g., planar flow).

In some embodiments, the impingement member 50 and/or nozzle 9 can be positioned so that the jet axis X′ is aligned with a center point of the impingement member 50 (such as shown in FIG. 3D) (e.g., a center point of the impingement surface 53), which may result in a redirection of the liquid jet 20 as a spray or mostly as a spray, in some embodiments. In other embodiments, the jet axis X′ can contact the impingement member 50 at a contact point offset from a center point of the impingement member 50 (e.g., superior to, inferior to, horizontally left of, horizontally right of, or any combination of these to the center point of the impingement member 50) and/or offset from a center point of the impingement surface 53 (e.g., superior to, inferior to, horizontally left of, horizontally right of, or any combination of these to the center point of the impingement surface 53). The contact point of the jet axis X′ with the impingement member 50 and/or impingement surface 53 can be affected by the horizontal (left or right) position of the nozzle 9, the vertical (inferior or superior) position of the nozzle 9, any horizontal (left or right) angular components of the jet axis X′, and any vertical (inferior or superior) angular components of the jet axis X′. Contact of the jet axis X′ offset from the center point of the impingement member 50 or impingement surface 53 may contribute to the formation of the stream 29 in the form of a liquid jet.

FIG. 11E depicts an axis Z′ and an axis Y extending through a center point 71 of the impingement surface 53. In some embodiments, the axis Z′ can be parallel or substantially parallel to the axis Z and/or the superior-inferior axis as shown in FIG. 11F. The axis Y can be perpendicular to the axis Z and may be parallel to the horizontal left-right axis as shown in FIG. 11F. In some embodiments, the axis Y can separate the impingement surface 53 into an upper vertical section and a lower vertical section. FIG. 11F shows an example of a contact point 72 of the liquid jet 20 with the impingement member 50 that may be beneficial for forming the stream 29 in the form of a liquid jet. In some embodiments, a radial offset of the contact point 72 from the center 71 of the impingement surface 53 can increase the amount of time a fluid contacts the surface of the impingement surface 53, which can create a vacuum to reduce apical pressure and to evacuate diseased material from the treatment region. In some embodiments, a radial offset of the contact point 72 from the center point 71 may also provide increased chaotic fluid motion, for example, by formation of the stream 29 in the form of a liquid jet. While some reduction in apical pressure may be desirable, in some embodiments, it is desirable to avoid applying excessive negative pressures to the tooth, which can cause pain to the patient. In some embodiments, a contact point 72 can be selected to produce a stream 29 in the form of a liquid jet and/or to provide a reduction in apical pressure without applying a negative apical pressure.

In some embodiments, the contact point 72 may be positioned at a radius between 0 inches and 0.063 inches from the center point 71. In some embodiments, the contact point 72 may be positioned at a radius of 0.010 inches to 0.05 inches from the center point 71. In some embodiments, the impingement surface 53 is hemispherical in shape. In some embodiments, a diameter of the inner edge of the hemispherical impingement surface 53 is 0.125 in. In some embodiments, the contact point 72 may be positioned at a distance from the center point 71 of between 1% and 49% of the diameter of the hemisphere, between 5% and 45% of the diameter of the hemisphere, between 8% and 40% of the diameter of the hemisphere, between 10% and 30% of the diameter of the hemisphere, between 15% and 25% of the diameter of the hemisphere, between 1% and 20% of the diameter of the hemisphere, between 5% and 25% of the diameter of the hemisphere, between 20% and 40% of the diameter of the hemisphere, between 25% and 45% of the diameter of the hemisphere, or any other suitable range. In some embodiments, it may be beneficial if the contact point 72 is offset from the center point 71 along the Y axis (e.g., horizontally offset to the left or right). In some embodiments, a vertical offset of the contact point without a horizontal offset may assist in producing a rotational flow about an axis parallel to the Y axis (e.g., vortex flow). In some embodiments, a horizontal offset without a vertical offset may assist in producing rotational flow about an axis parallel to the Z′ axis (e.g., swirling flow). In some embodiments, a contact point 72 offset both vertically and horizontally from the center point 71 can assist in producing rotational fluid motion about an axis having both vertical and horizontal components, which may, for example, provide characteristics of both vortex and swirling flows. In some embodiments, an axis of rotation of the rotational flow can be orthogonal to a plane created by the jet 20 and the return stream 29 in the form of a liquid jet. In some embodiments, an angle δ between the Z′ axis and a radial line extending from the center point 71 through the contact point 72 can be between −45° and 45°, between −30° and 30°, or between −15° and 15°.

In some embodiments, when contact point 72 is offset from the center point 71, the stream 29 in the form of a liquid jet will be redirected from the impingement member 50 at a position on the impingement surface 53 opposite the contact point 72. In some embodiments, the contact point 72 can be positioned superior to a vertical center of the impingement surface 53 (e.g., superior to the Y axis), and the stream 29 in the form of a liquid jet can be redirected from the impingement surface 53 inferior to the vertical center of the impingement surface (e.g., inferior to the Y axis), for example, as shown in FIG. 11D. In some embodiments, the contact point 72 can be positioned inferior to the vertical center of the impingement surface 53 (e.g., inferior to the Y axis), and the stream 29 in the form of a liquid jet can be redirected from the impingement surface 53 superior to the vertical center of the impingement surface (e.g., superior to the Y axis), for example, as shown in FIG. 11K. In some embodiments, the contact point 72 can be positioned lateral to a horizontal center of the impingement surface 53 (e.g. lateral to the Z′ axis) in a first lateral direction (for example, to the right of the horizontal center), and the s stream 29 in the form of a liquid jet can be redirected from the impingement surface 53 lateral to the horizontal center of the impingement surface in a second lateral direction (for example, to the left of the horizontal center). In some embodiments, the second liquid jet can be redirected from an opposite vertical and horizontal position of the impingement surface relative to the contact point 72. For example, with references to the axes Z′ and Y of FIG. 11E, a contact point 72 in an upper right quadrant may result in the stream 29 in the form of a liquid jet being redirected from the impingement surface 53 from the lower left quadrant.

In some embodiments, after impingement, the fluid from the jet 20 can spread out along the concave impingement surface 53 of the impingement member 50, and the impingement surface 53 can be shaped and/or angled such that the fluid recombines to emerge as the stream 29 in the form of a liquid jet. In some embodiments, the fluid can recombine to from the stream 29 in the form of a liquid jet on an opposite side of the impingement surface 53 from the contact point 72 of the jet 20. In some embodiments, fluid from the jet 20 can spread out into a plurality of fluid components along the impingement surface 53, and the fluid components can converge to recombine upon or after redirection from the impingement surface 53 as a stream 29 in the form of a liquid jet. In some embodiments, after converging to recombine as stream 29, the fluid components can diverge. For example, in some embodiments, the plurality of fluid components can be redirected to cross over one, and, upon intersecting one another, may temporarily form a second liquid jet.

For example, as shown in FIG. 11D, in some embodiments, the jet axis X′ may be aligned with a superior section of the impingement surface 53, so that the fluid from the fluid jet is biased to flow downward around the curved and/or angled sections of the impingement surface 53 to cause more of the redirected fluid (e.g., the stream 29 in the form of a liquid jet) to flow below the center of the impingement surface 53 and closer to the transition opening 30. In some embodiments, as explained above, the jet axis X′ can be disposed substantially perpendicular to the central axis Z (parallel to the axis X″). In some embodiments, the jet axis X′ can be angled relative to the central axis Z at an angle α in a range between 80° and 90°, in a range between 84° and 90°, or in a range between 86° and 90°. In some embodiments, the jet axis X′ can be angled relative to the central axis Z with an angle α of 80°, 81°, 82°, 83° 84° 85° 86°, 87° 88° or 89°. In some embodiments, the jet axis X′ can be angled relative to the axis X″ at an angle β in a range between 0° and 10°, in a range between 0° and 6°, or in a range between 0° and 4°. In some embodiments, the jet axis X′ can be angled relative to the axis X″ with an angle θ of 1°, 2°, 3°, 4°, 5°, 6°, 7°, 8° 9°, or 10°. Similarly, in some embodiments, the jet axis X′ can be offset horizontally (along the left-right axis) relative to the axis X″ by an angle equivalent to the angle β. In some embodiments, the jet axis X′ can be offset inferiorly relative to the axis X′, for example, as shown in FIG. 11K. In some embodiments, the jet axis X′ can be offset inferiorly from the axis X″ by the angle α.

In some embodiments, and as shown in FIG. 11D, both the jet axis X′ and the central axis X′″(and/or proximal facing edge of impingement member 50) may be positioned at an angle relative to the central axis Z and/or X″ axis. For example, in some embodiments, both jet axis X′ and the X′″ axis of impingement surface 53 may be positioned at an angle α relative to central axis Z or an angle β relative to the axis X″. In other embodiments, the X′″ axis may be offset at a different angle. In some embodiments, the axis X′″ may be offset inferiorly from the axis X″ by an angle between 0° and 10°, between 0° and 6°, or between 0° and 3°. In some embodiments, the axis X′″ may be offset inferiorly from the axis X″ by an angle of 1°, 2°, 3°, 4°, 5°, 6°, 7°, 8°, 9°, or 10°. The downward angle of the impingement surface 53 can cause a redirected fluid(e.g., the stream 29 in the form of a liquid jet) to return at the same angle relative to the axis X″. For example, if the axis X′″ is angled inferiorly form the axis X″ at an angle of 3°, a redirected fluid or jet (e.g., the s stream 29 in the form of a liquid jet) will return at an angle of 3° inferior to the axis X″ (e.g., downward towards transition opening 30). In some embodiments, it may be desirable that a maximum amount of the redirected flow (e.g., the stream 29 in the form of a liquid jet) flows over the transition opening 30. Redirection of the stream 29 in the form of a liquid jet downwards towards the transition opening may create increased fluid motion and/or more chaotic fluid motion.

In some embodiments, with the impingement member 50 having an impingement surface 53 in the form of a hemispherical recess as shown in FIG. 11D, when a fluid jet 20 impinges upon the impingement surface 53 at a contact point 72 offset from the center point 71 of its hemispherical recess, the fluid jet 20 may return from a side of the impingement surface 53 opposite the side it impinges upon. In some embodiments, passing of the fluid jet 20 and its redirected fluid or jet from the impingement member 50 may create a relative shear between the fluid jets. In some embodiments, nozzle 9 may be configured to direct the fluid jet 20 at impingement surface 53 horizontally offset from its center and cause a redirected fluid jet to return towards proximal chamber 60 also horizontally offset from its center. In some embodiments, it may be beneficial that the fluid jet 20 impinges upon the impingement surface 53 at a contact point superior to the Y axis to redirect the stream 29 in the form of a liquid jet downwards towards the transition opening. In some embodiments, at least a portion of the stream 29 in the form of a liquid jet may contact an inner wall of the distal chamber 70.

While the impingement member 50 is shown in the form of a hemisphere in FIGS. 11A-11J, in some embodiments, other shapes having a concave impingement surface 53 may be used to form a stream 29 in the form of a liquid jet after impingement of the jet 20 as described herein.

In some embodiments, the redirected fluid (e.g., the stream 29 in the form of a liquid jet) can induce fluid motion 24 within the distal chamber 70 when flowing over the transition opening 30 after impingement on the impingement member 50. In some embodiments, the fluid motion induced in the distal chamber 70 when the redirected fluid (e.g., stream 29 in the form of a liquid jet) flows over the transition opening 30 can include turbulent flow including vortices, cyclonic flow, and/or toroidal flow. In some embodiments, the fluid motion 24 induced in the distal chamber 70 when the redirected fluid or jet (e.g., stream 29 in the form of a liquid jet) flows over the transition opening 30 can be different at different times (e.g., toroidal flow at a first time and cyclonic flow at a second time), such that the flow profile in the distal chamber 70 can vary during the treatment procedure and/or be chaotic. In some embodiments, when the jet 20 impinges on or impacts the impingement member 50, fluid motion 24 is created along the impingement member 50 (e.g., along the one or more curved or angled surfaces, such as the impingement surface 53), along the interior surfaces of the proximal chamber 60, and/or within the fluid retained in the proximal chamber 60. Moreover, the movement of the jet 20 and/or the liquid stream diverted by the impingement member 50 can induce fluid motion 24 in the proximal chamber 60. In some embodiments, an interaction of the fluid of the jet 20 flowing towards the impingement member 50 and the fluid of the jet after redirection by the impingement member 50 (e.g., stream 29 in the form of a liquid jet) can induce fluid motion 24, for example, small vortices, turbulent flow, and/or chaotic flow. In some embodiments, some of the fluid motion 24 within the proximal chamber 60 can propagate into the distal chamber 70 to cause turbulence within the distal chamber 70, for example, by inducing shear stresses in the fluid in the distal chamber 70.

The combination of the different types of fluid motion 24 that can be generated by propagation and redirection of the jet 20 within the proximal chamber 60 can result in fluid motion 24 within the proximal chamber 60 and/or the distal chamber 70 that can be turbulent in nature and may rotate about multiple axes, which can increase the chaotic or turbulent nature of the flow and improve treatment efficacy. In some embodiments, the fluid motion 24 can propagate through the treatment region and can provide bulk fluid motion that flushes undesirable material (e.g., decayed organic matter) out of the treatment region. The combination of the fluid motion 24 and broadband generated pressure waves 23 can effectively remove undesirable materials of all shapes and sizes from large and small spaces, cracks, and crevices of the treatment region. In some embodiments, the fluid flow 24 can have sufficient momentum and structure to reach large and small spaces, cracks, and crevices of the treatment region. The fluid motion 24, which may be described as turbulent or unsteady, can include small eddies and may be non-repeating. Examples of fluid motion 24 that can occur within the fluid platform 2 are illustrated by arrows in FIG. 11D.

The combination of different types of fluid motion 24 can create unsteady flow such that, over the course of a treatment procedure, the fluid flow does not reach steady state. Some treatment instruments may induce fluid motion 24 in the treatment region that reaches a steady state after a time period. Steady flow can reduce treatment efficacy, for example, because the flow vectors of the treatment fluid do not change sufficiently so as to reach small untreated spaces that may be located along non-linear tubules or other spaces or cracks. Beneficially, the arrangement of the pressure wave/fluid motion generator 10, impingement member 50, the proximal chamber 60, and the distal chamber 70 can cooperate to generate non-steady flow during operation in a treatment procedure. Non-steady flow can create changing flow direction and/or changing flow vectors that increase the probability that, over the course of the treatment, the treatment fluid will reach remote regions that would otherwise be difficult or impossible to reach with steady state operational devices.

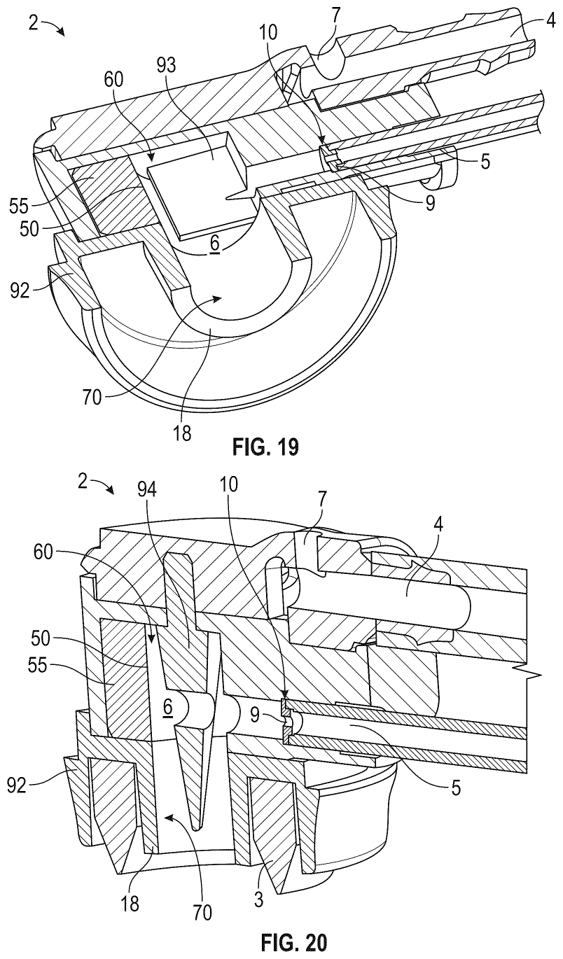

In some embodiments, the fluid platform 2 may include one or more vibrating or oscillatory members that can be shaped, sized, positioned, and/or otherwise configured to amplify an amplitude of one or more frequencies of pressure waves within the chamber. Further details regarding vibrating or oscillatory members are discussed with respect to FIGS. 18 and 19, which depict an example of a vibrating or oscillatory member in the form of a clapper 93.

As shown in the embodiment of the fluid platform 2 of FIG. 11A through FIGS. 11C-11F, in certain embodiments, the impingement member 50 may be a separate piece that can be positioned within the proximal chamber 60. Alternatively, the impingement member 50 may be a curved or angled sidewall of the proximal chamber 60 (e.g., the impingement member 50 may be integrally or monolithically formed with the wall of the proximal chamber 60).

The fluid platform 2 can also include an evacuation or outlet line 4 to convey waste or effluent liquids to a waste reservoir, which may be located, for example, in a system console 102. A suction port 8 or fluid outlet can be exposed to the proximal chamber 60 along a wall of the proximal chamber 60 offset from the central axis Z. For example, as shown in FIG. 11D, the suction port 8 can be disposed along an upper wall of the proximal chamber 60 opposite the transition opening 30. A vacuum pump (not shown) can apply vacuum forces along the outlet line 4 to draw waste or effluent liquids 19 out of the proximal chamber 60 through the suction port 8, along the outlet line 4, and to the waste reservoir. In some embodiments, only one suction port 8 can be provided. In other embodiments, the fluid platform 2 can include a plurality (e.g., two) suction ports positioned laterally opposite one another. In some embodiments, more than two suction ports can be provided. In some embodiments, the drawing of fluid out of the proximal chamber 60 by the suction port 8 can affect the fluid motion 24 in the proximal chamber 60. For example, the action of the suction port 8 can withdraw at least some fluid from the liquid jet 20 that has passed back over the transition opening 30 after impingement on the impingement member 50. In some embodiments, this action of the suction port may prevent or reduce stagnation within the fluid in the proximal chamber 60 and/or may contribute to turbulent or chaotic fluid motion as described herein.

As shown in FIGS. 11C-11D, in some embodiments, the outlet line 4 and pressurized fluid inlet line 5 can be part of a separate manifold 80 that can couple to main body 40 to form the fluid platform 2. The vent 7 may also be positioned in the manifold 80. The main body 40 and manifold 80 may together form chamber 6. In some embodiments, the main body 40 and manifold 80 may together form proximal chamber 60 of chamber 6, and the main body 40 alone may form transition opening 30 and distal chamber 70. The main body 40 may include access port 18, flange 16, and sealing cap 3 as described herein.

The impingement member 50 may be captured between the manifold 80 and the main body 40. For example, the impingement member may include an outer flange for securing within fluid platform 2. The main body 40 may be coupled to manifold 80 by being press fit into manifold 80. In some embodiments, the main body 40 and manifold 80 may form a cavity for holding impingement member 50 in place. Further, in some embodiments, impingement member 50 may be held in place at its posterior end (facing proximal chamber 60) by the structure of main body 40 and at its anterior end (facing away from proximal chamber 60) by the structure of manifold 80. The impingement member 50 may be metallic, ceramic, or formed of any other suitable material for receiving and redirecting the fluid jet 20.

Further as shown in FIG. 11D, the proximal chamber 60 and the distal chamber 70 can each be generally cylindrical in shape. A longitudinal axis of the cylindrical proximal chamber 60 (which in the illustrated embodiment may be coextensive or parallel to the anterior-posterior axis and/or at an angle relative to the jet axis X′) can extend perpendicularly to a longitudinal axis of the cylindrical distal chamber 70 (which in the illustrated embodiment may be coextensive or parallel with the central axis Z). As shown in FIG. 11D, the proximal chamber 60 and distal chamber 70 may have different geometries and/or volumes. In the illustrated embodiment, the impingement member 50 is disposed longitudinally beyond the transition opening 30 along the jet axis X′ such that the transition opening 30 is longitudinally between the impingement member 50 and the nozzle 9 along the jet axis X′. In some embodiments, a jet length (i.e., a distance between the nozzle and an impingement point) can be between 1 mm and 20 mm, between 3 mm and 10 mm or any other suitable length. In some embodiments, a diameter of the proximal chamber 60 can be between 0.1 mm and 20 mm, between 1 mm and 10 mm, or any other suitable diameter. In some embodiments, a diameter of the distal chamber 70 can be between 0.5 mm and 10 mm, between 2 mm and 5 mm, or any other suitable diameter. In some embodiments, a height of the distal chamber 70 can be between 0 mm and 20 mm, between 0 mm and 6 mm or any other suitable height.

As shown in FIG. 11D, the proximal chamber 60 can accordingly have a first interior surface geometry 26a bounded by at least a wall 28a extending along upper, lower, and side surface(s) of the proximal chamber 60 and the impingement member 50. The distal chamber 70 can have a second interior surface geometry 26b bounded by at least a wall 28b extending along side surface(s) of the distal chamber 70. The first and second interior surface geometries 26a, 26b can be different as shown. For example, the first interior surface geometry 26a can comprise a curved surface (e.g., an approximately cylindrical surface) extending at an angle relative to or substantially parallel to the jet axis X′ from the nozzle 9 (or a location distal the nozzle 9) to the impingement surface 53 of the impingement member 50. By contrast, the second interior surface geometry 26b can comprise a curved surface (e.g., an approximately cylindrical surface) extending distally along the central axis Z. The transition opening 30 can comprise a discontinuity that provides a non-uniform or abrupt flow transition between the proximal and distal chambers 60, 70. The discontinuity provided by the transition opening 30 and the differing interior surface geometries 26a, 26b can beneficially create unsteady flow of treatment fluid during operation of the treatment instrument in a treatment procedure. Non-uniform transitions can include asymmetric structures or irregularities in a transition region. The transition region can include the transition opening 30 and portions of the proximal chamber 60 and distal chamber 70 adjacent the transition opening 30. The asymmetric structures or irregularities may include one or more offsets, steps, recesses, or any other suitable structures.

In some embodiments, a ratio of a volume of the proximal chamber 60 to a volume of the distal chamber 70 is between 7:4 and 15:2. In some embodiments, a ratio of a volume of the proximal chamber 60 to a circumference of the transition opening 30 is between 1:150 and 1:20. In some embodiments, a ratio of a jet distance to a volume of the proximal chamber 60 is between 10:1 and 50:1. In some embodiments, a ratio of a jet distance to a jet height is between 2:1 and 13:2.

In some embodiments, the fluid platform 2 may include one or more additional fluid inlets, for example, for providing a filling material or filling material component. Additional fluid inlets may be positioned, for example, below the inlet 5 or below the impingement member 50. Additional details regarding embodiments with additional fluid inlets can be found throughout U.S. patent application Ser. No. 16/894,667, the entire contents of which are incorporated by reference herein in its entirety and for all purposes.

Additional details regarding fluid platforms can be found throughout U.S. patent application Ser. No. 16/879,093, the entire contents of which are incorporated by reference herein in its entirety and for all purposes.

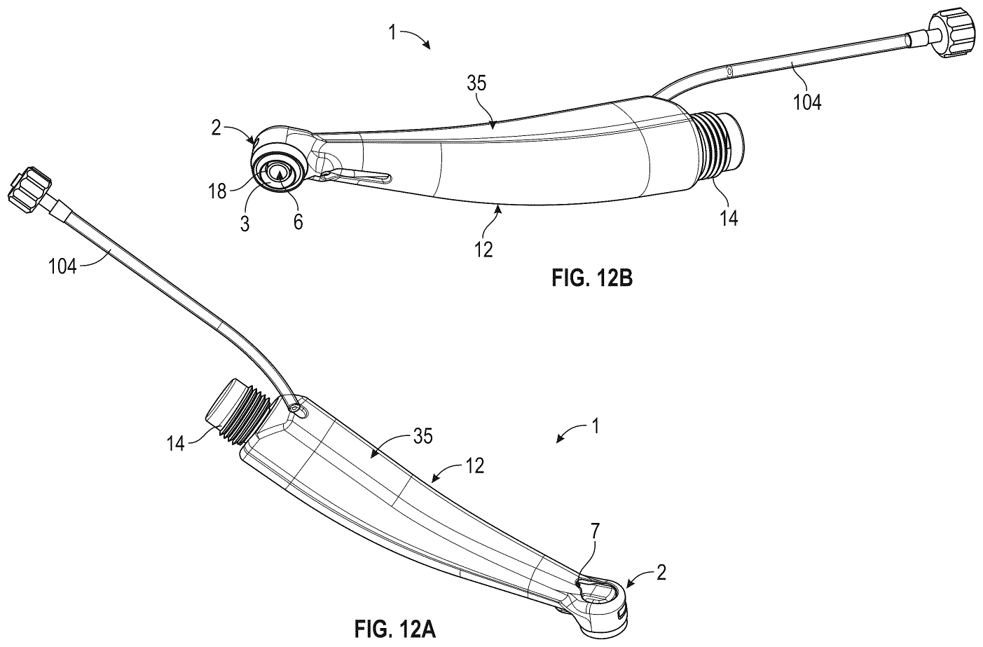

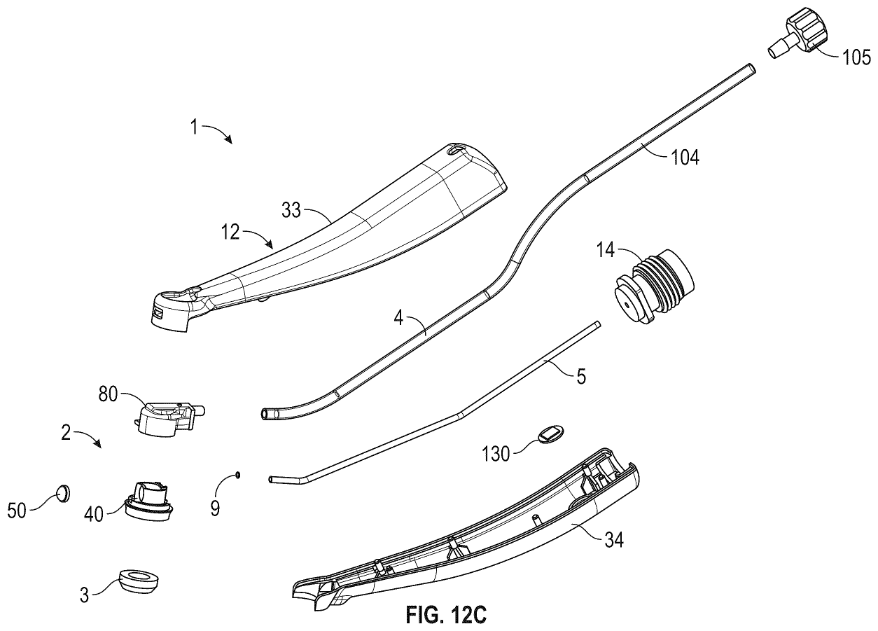

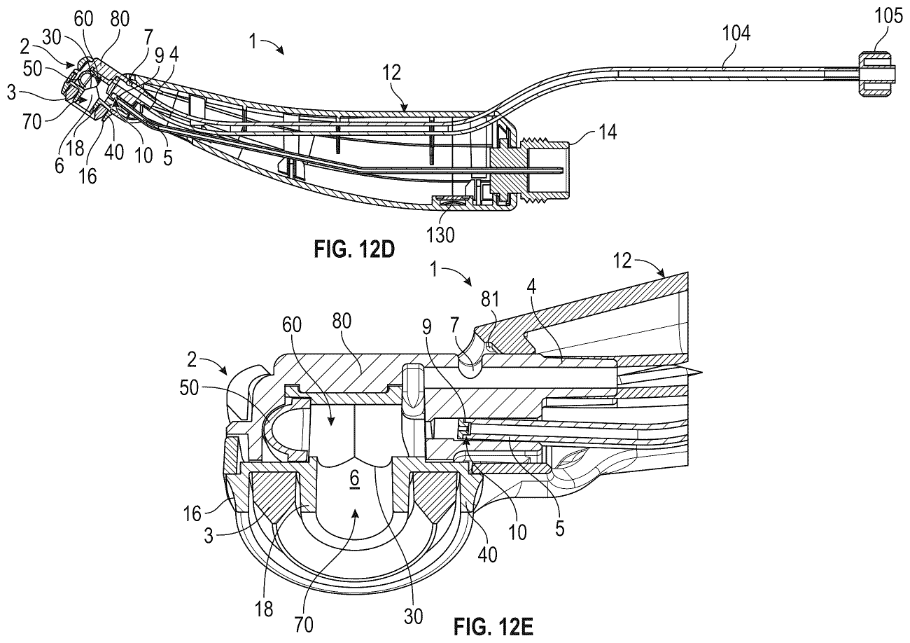

FIGS. 12A-12E illustrate another embodiment of a treatment instrument 1. In particular, FIG. 12A is a top perspective view of a treatment instrument 1 according to one embodiment. FIG. 12B is a bottom perspective view of the treatment instrument 1 of FIG. 12A. FIG. 12C is a top perspective exploded view of the treatment instrument of FIG. 12A. FIG. 12D is a side sectional view of the treatment instrument of FIG. 12A. FIG. 12E is a magnified bottom perspective sectional view of the fluid platform of the treatment instrument of FIG. 12A.

The treatment instrument 1 of FIGS. 12A-12E may include a handpiece 12 sized and shaped to be gripped by the clinician. The treatment instrument 1 can further include a fluid platform 2. As shown in FIGS. 12A-12E, the fluid platform 2 may be the embodiment of the fluid platform 2 depicted in FIGS. 11A-J. The fluid platform 2 can be coupled to a distal portion of the handpiece 12. As explained herein, in some embodiments, the fluid platform 2 can be removably connected to the handpiece 12. In other embodiments, the fluid platform 2 can be non-removably attached to the handpiece 12 or can be integrally formed with the handpiece 12. In still other embodiments, the fluid platform 2 may not couple to a handpiece and may instead serve as a treatment cap that is adhered (or otherwise coupled or positioned) to the tooth without using a handpiece. As shown in FIGS. 12A-12B, an interface member 14 can be provided at a proximal end portion of the handpiece 12, which can removably couple to one or more conduits to provide fluid communication between a console 102 as described herein and the treatment instrument 1.

As shown in FIGS. 12A, and as described herein, a vent 7 can be provided through a portion of the handpiece 12 to provide fluid communication between an outlet line 4 (which can comprise one of the at least one conduits 104 described herein and/or a portion of the fluid platform 2) and ambient air. As explained herein, the vent 7 can serve to regulate the pressure in the fluid platform 2 and can improve the safety and efficacy of the treatment instrument 1. As shown in FIG. 12B, an access port 18 can be provided at a distal portion of the fluid platform 2 to provide fluid communication between a chamber 6 defined by the fluid platform 2 and the treatment region of the tooth 110. For example, as explained above with respect to FIG. 1A, in root canal cleaning procedures, a sealing cap 3 at the distal portion of the fluid platform 2 can be positioned against the tooth 110 over the access opening 118 to provide fluid communication between the chamber 6 and the interior of the tooth 110 (e.g., the pulp cavity 111 and root canal(s) 113). In other embodiments, as explained above with respect to FIG. 1B, the sealing cap 3 can be positioned against the tooth 110 over the carious region at an exterior surface 119 of the tooth 110 to provide fluid communication between the chamber 6 and the carious region to be treated.

As shown in FIG. 12C, the handpiece 12 may include a top shell 33 and a bottom shell 34. The top shell 33 and bottom shell 34 can be coupled together to form a handpiece body 35. In some embodiments, the top shell 33 and bottom shell 34 can be removably coupled to one another. In other embodiments, the top shell 33 and bottom shell 34 can be non-removably attached to one another or integrally formed with one another. The handpiece body 35 can house an inlet line 5 and an outlet line 4 of the treatment instrument 1, a communications chip 130, and the fluid platform 2. In some embodiments, at least a portion of the inlet line 5 and/or at least a portion of the outlet line 4 can be formed in the fluid platform 2. In some embodiments, the communications chip can be configured to be programmed with information about the particular handpiece 12 to which the communications chip is coupled. The communications chip 130 can be configured to communicate with a wireless reader. The communications chip 130 may be an RFID chip. Additional examples of communications chips and wireless readers are described through U.S. Pat. No. 9,504,536, the entire contents of which are incorporated by reference herein in their entirety and for all purposes. The handpiece 12 may also include a connector 105 that fluidly connects the outlet line 4 with a console. An interface member 14 can be provided at a proximal end portion of the handpiece 12, which can removably couple to the one or more conduits 104 to provide fluid communication between the console 102 and the treatment instrument 1.

As shown, the fluid platform 2 may include a manifold 80, a main body 40, a nozzle 9, an impingement member 50, and a sealing cap 3.

FIGS. 12D-12E show how components of treatment instrument 1 and fluid platform 2 may connect and integrate with one another according to some embodiments. The inlet line 5 may be disposed at a proximal end of the manifold 80 of the fluid platform 2 and may include a nozzle 9 to form the pressure wave generator 10 (which is also referred to as a fluid motion generator herein). The pressure wave generator 10 may be in fluid communication with the chamber 6 of fluid platform 2. The chamber 6 of fluid platform 2 may include a proximal chamber 60 and a distal chamber 70 fluidly connected to one another through a transition opening 30. The impingement member 50 may be disposed within the proximal chamber 60 opposite (e.g., distal to) pressure wave generator 10. The outlet line 4 may be fluidly connected to the chamber 6 of main body 40 through the manifold 80 and may fluidly connect to the vent 7. The distal chamber 70 may fluidly connect to a treatment region of a tooth 110 via an access port 18 of main body 40. The sealing cap 3, which may be coupled to the main body 40 by a flange 16 or connected to or formed with the fluid platform 2 as otherwise described herein, may be disposed around the access port 18 and substantially fluidly seal the chamber 6 with the treatment region of tooth 110, for example, when the clinician presses the sealing cap 3 against the tooth 110 over the treatment region. In some embodiments, the handle 12 can include a recess 81 positioned above the vent 7. The recess 81 can be positioned, shaped, and or otherwise configured to prevent blockage or occlusion of the vent 7. For example, in some embodiments, the recess 81 can allow the vent 7 to communicate with an interior of the handle 12 if the portion of the portion of the handle 12 above the vent 7 is covered, for example, by a finger over the distal end of the handle 12. In some embodiments, the recess 81 can provide a vent pathway between the vent 7 and the interior of the handle 12.

The dental treatments disclosed herein can be used with any suitable type of treatment fluid, e.g., cleaning fluids. In filling procedures, the treatment fluid can comprise a flowable filling material that can be hardened to fill the treatment region. The treatment fluids disclosed herein can be any suitable fluid, including, e.g., water, saline, etc. In some embodiments, the treatment fluid can be degassed, which may improve cavitation and/or reduce the presence of gas bubbles in some treatments. In some embodiments, the dissolved gas content can be less than about 1% by volume. Various chemicals can be added to treatment solution, including, e.g., tissue dissolving agents (e.g., NaOCl), disinfectants (e.g., chlorhexidine), anesthesia, fluoride therapy agents, EDTA, citric acid, and any other suitable chemicals. For example, any other antibacterial, decalcifying, disinfecting, mineralizing, or whitening solutions may be used as well. Various solutions may be used in combination at the same time or sequentially at suitable concentrations. In some embodiments, chemicals and the concentrations of the chemicals can be varied throughout the procedure by the clinician and/or by the system to improve patient outcomes.

In some systems and methods, the treatment fluids used can comprise degassed fluids having a dissolved gas content that is reduced when compared to the normal gas content of the fluid. The use of degassed treatment fluids can beneficially improve cleaning efficacy, since the presence of bubbles in the fluid may impede the propagation of acoustic energy and reduce the effectiveness of cleaning. In some embodiments, the degassed fluid has a dissolved gas content that is reduced to approximately 10%-40% of its normal amount as delivered from a source of fluid (e.g., before degassing). In other embodiments, the dissolved gas content of the degassed fluid can be reduced to approximately 5%-50% or 1%-70% of the normal gas content of the fluid. In some treatments, the dissolved gas content can be less than about 70%, less than about 50%, less than about 40%, less than about 30%, less than about 20%, less than about 10%, less than about 5%, or less than about 1% of the normal gas amount. In some embodiments, the degassed fluids may be exposed to a specific type of gas, such as ozone, and carry some of the gas (e.g., ozone) with them into the treatment region, for example, in the form of gas bubbles. At the treatment region, the gas bubbles expose the treatment region to the gas (e.g., ozone) for further disinfection of the region.

### Additional Examples of Fluid Platforms and Components

Additional examples of fluid platforms, components, and features thereof, aspects of which may be used with, combined with, and/or substituted with the various aspects of embodiments of the treatment instruments 1 and fluid platforms 2 described herein, are described with respect to FIGS. 13-38. Unless otherwise noted, components of FIGS. 13-38 may be generally similar to or the same as like-numbered components of FIGS. 2D-2K, FIGS. 3A-3H, FIGS. 4A-4E, FIGS. 5A-5E, FIGS. 6A-6B, FIGS. 7A-7F, FIGS. 8A-8F, FIGS. 9A-9B, FIGS. 10A-10J, and FIGS. 11A-11J.

FIG. 13 shows a top perspective sectional view of a fluid platform 2 according to some embodiments. As shown, in some embodiments a fluid platform 2 may include a guide tube 91 coupled to and fluidly connected to an inlet line 5 that opens into a chamber 6 of the fluid platform 2. The guide tube 91 may include a nozzle 9 for generating a liquid jet 20. The guide tube 91 may extend inferiorly (distally) into the chamber 6 from a superior end of the chamber 6. For example, a central axis of the guide tube 91 may be substantially aligned with a superior-inferior axis (as shown, for example, in FIG. 11F). In other embodiments, the guide tube 91 may extend into the chamber 6 in other directions. In some embodiments, the fluid platform 2 can include an impingement member 50. The impingement member 50 may be positioned opposite the guide tube 91 within the chamber 6 of the fluid platform 2 as shown in FIG. 13. The impingement member 50 may be positioned so as to be impinged upon by the fluid jet 20 from the nozzle 9. In some embodiments, the impingement member 50 may be removably couplable to (e.g., attachable to and/or detachable from) the fluid platform 2. In some embodiments, the impingement member 50 may be removably couplable to (e.g., attachable to and/or detachable from) the guide tube 91. In some embodiments, the impingement member 50 may include radially outward extending supports that secure the impingement member 50 within the chamber 6. In some embodiments, the radially outward extending supports of the impingement member 50 may also allow for the flow of fluid between adjacent supports. In some embodiments, the fluid platform 2 can be formed in three pieces including a first housing member housing the guide tube 91, the inlet line 5, and the outlet line 4, a second piece forming the chamber 6, and the impingement member 50.

FIG. 14 is a top perspective sectional view of a fluid platform 2 according to some embodiments. The fluid platform 2 may comprise a manifold 80, a main body 40, and a bottom cap 92. In some embodiments, the fluid platform 2 includes an impingement ring 55 with an impingement member 50 adjacent a proximal chamber 60 formed by the manifold 80 and main body 40. The bottom cap 92 may form a distal chamber 70 in fluid communication with proximal chamber 60. In some embodiments, an inner diameter of the distal chamber 70 substantially matches an inner diameter of the impingement ring 55 and proximal chamber 60. In some embodiments, an inner diameter of the impingement ring can be between 2 mm to 5 mm, between 3 mm to 4 mm, 3.5 mm or about 3.5 mm. In some embodiments, an inner diameter of the distal chamber 70 can be between 2 mm to 5 mm, between 3 mm to 4 mm, 3.5 mm or about 3.5 mm. In some embodiments, an inner diameter of the proximal chamber 60 can be between 2 mm to 5 mm, between 3 mm to 4 mm, 3.5 mm or about 3.5 mm.

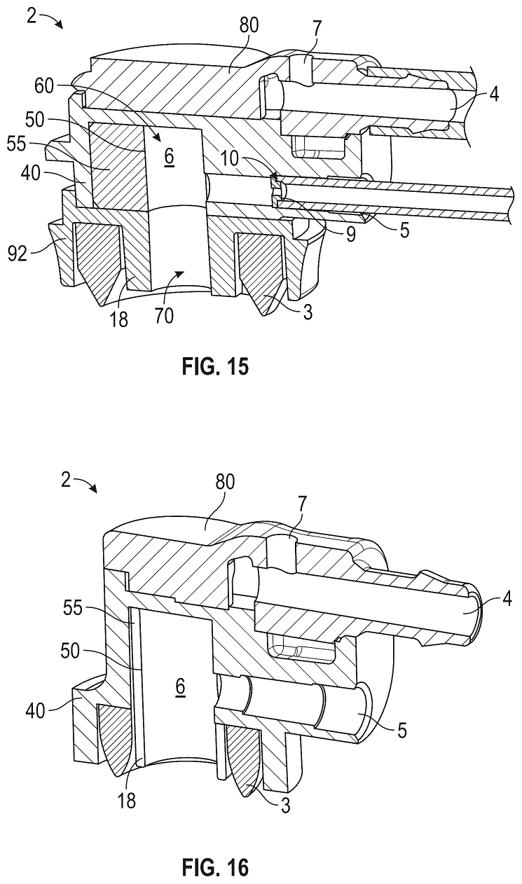

FIG. 15 is a top perspective sectional view of a fluid platform 2 according to some embodiments. Similar to the embodiment of FIG. 14, the fluid platform 2 may comprise a manifold 80, a main body 40, and a bottom cap 92. In some embodiments, the fluid platform 2 includes an impingement ring 55 with an impingement member 50 adjacent a proximal chamber 60 formed by the manifold 80 and main body 40. The bottom cap 92 may form a distal chamber 70 in fluid communication with proximal chamber 60. In some embodiments, an inner diameter of the distal chamber 70 substantially matches an inner diameter of the impingement ring 55 and proximal chamber 60. In some embodiments, an inner diameter of the impingement ring is between 1.5 mm to 4.5 mm, between 2.5 mm to 3.5 mm, 3.0 mm or about 3.0 mm. In some embodiments, an inner diameter of the distal chamber 70 is between 1.5 mm to 4.5 mm, between 2.5 mm to 3.5 mm, 3.0 mm or about 3.0 mm. In some embodiments, an inner diameter of the proximal chamber 60 is between 1.5 mm to 4.5 mm, between 2.5 mm to 3.5 mm, 3.0 mm or about 3.0 mm.

FIG. 16 is a top perspective sectional view of a fluid platform 2 according to some embodiments. As shown, in some embodiments an impingement ring 55 of the fluid platform 2 may comprise a thin wall and provide a continual surface from a superior to an inferior side of a chamber 6 within the fluid platform 2. In some embodiments, the inferior end of the impingement ring 55 forms the access port 18. In some embodiments, a sealing cap 3 may be disposed between an outer surface of the impingement ring 55 and a portion of a main body 40. In some embodiments, the configuration of the embodiment shown in FIG. 16 may have smaller dimensions and features than other embodiments described herein. In some embodiments, the dimensions of the fluid platform 2 of FIG. 16 may be beneficial to form a seal with an anterior tooth and/or relatively smaller teeth.

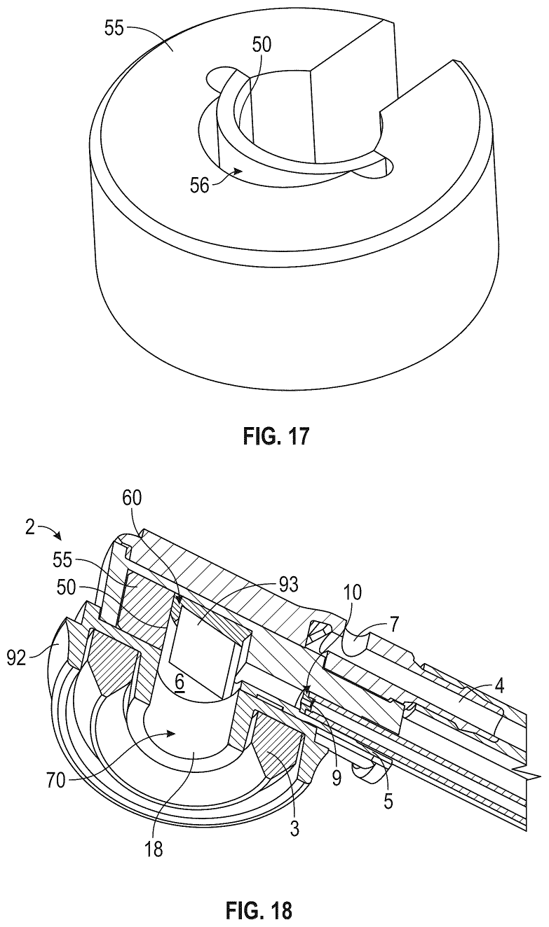

FIG. 17 is a perspective view of an impingement ring 55 according to some embodiments. As shown, in some embodiments the impingement ring 55 may comprise an impingement member 50. The impingement member 50 may extend across a central region of the impingement ring. In some embodiments, when inside a fluid platform 2, the impingement member 50 may be disposed over (e.g., superior to or proximal to) an access port 18 of the fluid platform 2. In some embodiments, the impingement member 50 can be spaced apart from an inner wall section of the impingement ring to allow for suction/evacuation of fluid in a region or port 56 of the impingement ring. For example, the suction port 56 may be in fluid communication with one or more suction ports 8 disposed anterior to the impingement element 50 (e.g., between the impingement member 50 and an anterior inner wall section of the impingement ring) when the impingement ring 55 is positioned within a fluid platform 2. In some embodiments, the impingement ring 55 may include one or more suction ports 56 disposed anterior to the impingement element 50. In some embodiments, the impingement member 50 of the impingement ring 55 may be disposed approximately over a superior-inferior central axis Z of a fluid platform 2 as described herein (e.g., approximately over a central superior-inferior central axis of an access port 18) with a suction port 56 disposed anterior to the impingement element 50 and fluidly connected to the access port 18.

FIG. 18 is a bottom perspective sectional view of a fluid platform 2 according to some embodiments. In some embodiments the fluid platform 2 may comprise a divider or clapper 93 disposed within a proximal chamber 60 of a chamber 6 of the fluid platform 2. The clapper 93 may be a substantially planar element that may be attached to an inner wall (e.g., a posterior inner wall) of the proximal chamber 60. In some embodiments, when attached to a posterior inner wall, the clapper 93 can extend in a substantially anterior direction. The clapper 93 may extend completely across the proximal chamber 60, or, in some embodiments, may extend only partially across the proximal chamber 60. In some embodiments, the clapper 93 may be substantially aligned with a plane formed by a superior-inferior axis and an anterior-posterior axis of the fluid platform 2 as described herein. In some embodiments, the clapper 93 may be positioned at least partially within a path of a liquid jet from the nozzle 9. In other embodiments, the clapper 93 can be offset from the path of the liquid jet. In some embodiments, the clapper 93 may be rigid. In other embodiments, the clapper 93 may be semi-rigid and can move in response to contact of a fluid jet 20 with the divider or in response to fluid motion in the proximal chamber 60. In some embodiments, the clapper 93 may provide for modified fluid motion inside the proximal chamber 60 of the fluid platform 2 (e.g., in response to contact of the fluid jet 20 with the divider or contact of fluid redirected from the impingement member 50 or otherwise moving within the chamber 60 with the divider). In some embodiments, the clapper 93 may comprise sheet metal (e.g., 0.001″ sheet metal). In some embodiments, the nozzle 9 of the fluid platform 2 of FIG. 18 may have a 68 μm opening.

In some embodiments, the clapper 93 may be a vibrating or oscillatory member. The clapper 93 can be configured to oscillate to amplify at least one frequency of pressure waves within the chamber 6. For example, in certain embodiment, the pressure waves may include a range of frequencies that are effective for cleaning a treatment region of the tooth (e.g., a root canal). The clapper 93 can be configured to (e.g., shaped, dimensioned, positioned, etc.) to amplify an amplitude of at least one frequency in the range of frequencies effective for cleaning a treatment region. For example, in some embodiments, the clapper 93 can be configured to oscillate at a natural frequency that corresponds to at least one frequency effective for cleaning a treatment region of the tooth. Amplification of an amplitude of an effective frequency may increase the effectiveness of pressure waves produced by the fluid platform. In some embodiments, the clapper 93 can be configured to oscillate in response to fluid motion in the chamber 6 (e.g., fluid motion created by a liquid jet 20 and/or fluid redirected from an impingement member, for example, in the form of a second liquid jet).

While a single clapper is shown in FIG. 18, some embodiments, may include a plurality of vibrating or oscillatory members. In some embodiments, different oscillatory members can be configured to amplify different frequencies in a range of frequencies of the pressure waves that are effective for cleaning a treatment region. For example, the fluid platform 2 can include a plurality of oscillatory members having different natural frequencies. The natural frequencies of the oscillatory members can be tuned by modifying the shape, size, and/or material of the oscillatory members to have desired frequency characteristics.

In some embodiments, the fluid platform 2 can include a plurality of vibrating or oscillatory members having different shapes and/or sizes, which may provide different natural frequencies and/or amounts of amplification. In some embodiments, an oscillatory member may cantilevered, tubular, elongate, or any other suitable shape.

In some embodiments, a plurality of oscillatory members may be positioned at different locations exposed to the chamber 6. Different locations may affect the amount of amplification provided by the oscillatory members. In some embodiments, an oscillatory member may positioned at the transition opening between the proximal chamber 60 and distal chamber 70 (e.g., extending from a posterior side of the transition opening). In other embodiments, an oscillatory member can extend from a posterior wall of the proximal chamber 60, an anterior wall of the proximal chamber 60, a side wall of the proximal chamber 60, a superior wall of the proximal chamber 60, and/or inferior wall of the proximal chamber 60, within the distal chamber 70, or at any other suitable location.[0249] FIG. 19 is a bottom perspective sectional view of a fluid platform 2 according to some embodiments. Similar to the embodiment of FIG. 18, in some embodiments the fluid platform 2 may comprise a divider or clapper 93 disposed within a proximal chamber 60 of a chamber 6 of the fluid platform 2. The clapper 93 may be a substantially planar element that may be attached to an inner wall (e.g., a posterior inner wall) of the proximal chamber 60. In some embodiments, when attached to a posterior inner wall, the clapper 93 can extend in a substantially anterior direction. The clapper 93 may extend completely across the proximal chamber 60, or, in some embodiments, may extend only partially across the proximal chamber 60. In some embodiments, the clapper 93 may be substantially aligned with a plane formed by a superior-inferior axis and an anterior-posterior axis of the fluid platform 2 as described herein. In some embodiments, the clapper 93 may be positioned at least partially within a path of a liquid jet from the nozzle 9. In other embodiments, the clapper 93 can be offset from the path of the liquid jet. In some embodiments, the clapper 93 may be rigid. In other embodiments, the clapper 93 may be semi-rigid and can move in response to contact of a fluid jet 20 with the divider or in response to fluid motion in the proximal chamber 60. In some embodiments, the clapper 93 may provide for modified fluid motion inside the proximal chamber 60 of the fluid platform 2 (e.g., in response to contact of the fluid jet 20 with the divider or contact of fluid redirected from the impingement member 50 or otherwise moving within the chamber 60 with the divider). In some embodiments, the clapper 93 may be formed and/or molded with the fluid platform 2. In some embodiments, the nozzle 9 of the fluid platform 2 of FIG. 19 may have a 68 μm opening. As described with respect to FIG. 18, in some embodiments, the clapper 93 can be an oscillatory member.

FIG. 20 is a top perspective sectional view of a fluid platform 2 according to some embodiments. In some embodiments, the fluid platform 2 may comprise a fluidic modifier 94. The fluidic modifier 94 may be positioned within a center or central region of a chamber 6 of the fluid platform 2. In some embodiments, the fluidic modifier 94 may extend inferiorly within the central region (e.g., from an upper inner wall of the chamber 6, such as from a superior inner surface of the chamber 6). The fluidic modifier 94 may comprise a generally cone-like shape or taper extending (e.g., inferiorly) within the chamber 6 of the fluidic platform 2. In other embodiments, the fluidic modifier 94 may be generally cylindrical in shape. In some embodiments, the fluidic modifier 94 may extend inferiorly across at least a portion of a proximal chamber 60 of the fluid platform 2. In some embodiments the fluidic modifier 94 may extend inferiorly across the proximal chamber 60 and additionally across at least a portion of a distal chamber 70 of the fluid platform 2 as shown. The fluidic modifier 94 may attach to or be formed with the fluid platform 2. As shown in FIG. 20, in some embodiments, the fluidic modifier 94 may comprise a through hole or cross hole positioned to allow a liquid jet 20 from the nozzle 9 to substantially pass through the through hole. For example, the through-hole may be substantially aligned with a central axis of a nozzle 9 of the fluidic platform 2 or substantially aligned with a jet axis of a jet produced by the nozzle 9. In some embodiments of a fluid platform 2 comprising a fluidic modifier 94, a liquid jet 20 may leave the nozzle 9, pass through the through hole of the fluidic modifier 94, and impinge upon an impingement member 50. In some embodiments, the fluidic modifier 94 may modify the fluid motion of the fluid platform 2.

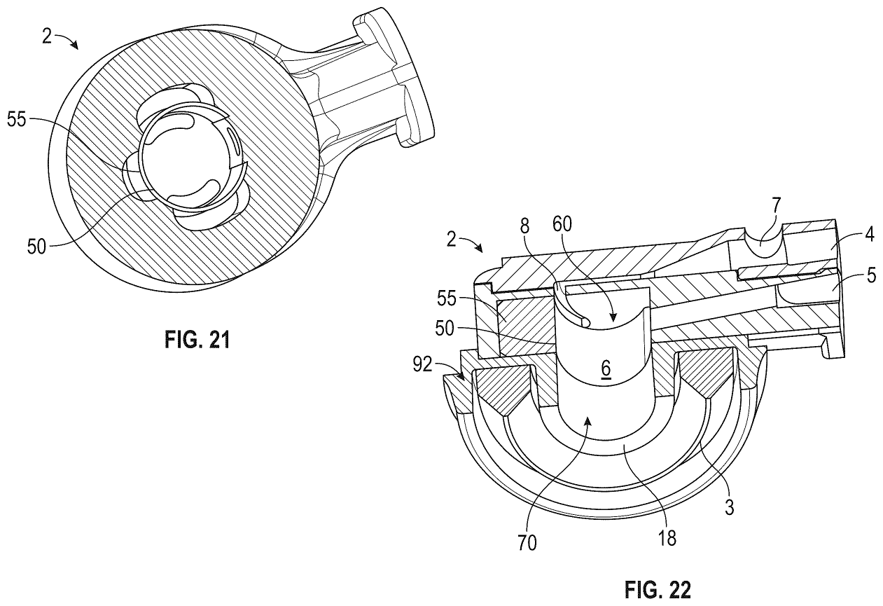

FIG. 21 is a bottom perspective view of an impingement ring 55 in a fluid platform according to some embodiments. As shown, in some embodiments an impingement ring 55 with an impingement element 50 may comprise a thin walled and flexible structure supported at multiple (e.g., 3, 4, or more) points of contact within the fluid platform 2. In this configuration, the impingement ring 55 may act like a drum to amplify pressure waves and/or sonics within the fluid platform 2. Further, the impingement ring 55 may vibrate to amplify and/or transmit pressure waves and/or sound waves to the treatment region of a tooth 110.

FIG. 22 is a bottom perspective sectional view of a fluid platform 2 according to some embodiments. As shown in FIG. 22, in some embodiments the fluid platform 2 may comprise a suction port 8 and at an anterior end of a proximal chamber 60 of the fluid platform 2 (e.g., at an upper inner wall of the proximal chamber 60). The suction port 8 can be positioned on an opposite side of the chamber from the fluid inlet 5. In some embodiments, the suction port 8 in this configuration may allow for efficient flow of waste or effluent fluids from the fluid platform 2 and reduced apical pressure.

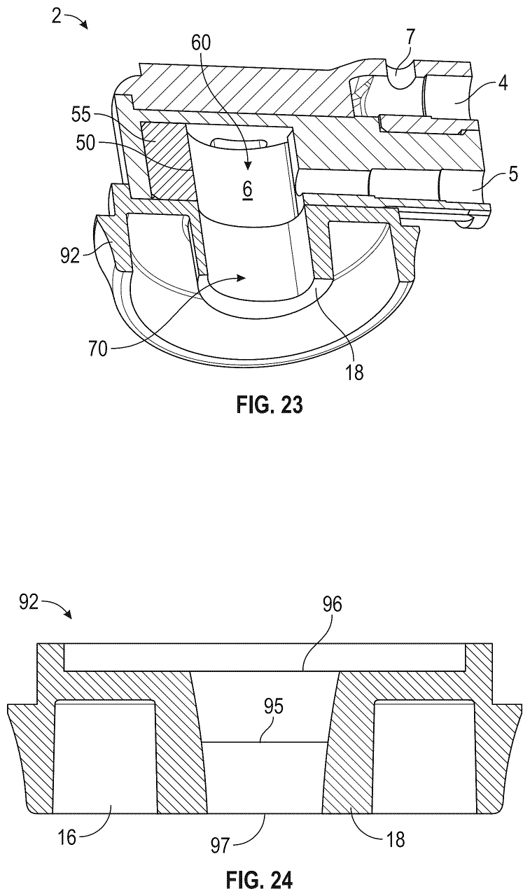

FIG. 23 is a bottom perspective sectional view of a fluid platform 2 according to some embodiments. As shown and as described herein, in some embodiments a chamber 6 including a proximal chamber 60 and a distal chamber 70 of the fluid platform 2 may include an elliptical cross-sectional shape (e.g., an elliptical shape when a cross-section is taken along a plane formed by an anterior-posterior axis and a left-right axis as shown relative to FIG. 11F). In some embodiments, only one of the proximal chamber 60 and the distal chamber 70 has an elliptical cross-sectional shape. In some embodiments, the proximal chamber 60 and distal chamber 70 may each have elliptical cross-sectional shapes that differ from one another (e.g., in size and/or orientation). In some embodiments an impingement ring 55 may have an elliptical cross-sectional shape. In some embodiments, the elliptical cross-sectional shape of the impingement ring can substantially match an elliptical cross-sectional shape of the distal chamber 70. The elliptical cross-sectional shapes of the proximal chamber 60, distal chamber 70, and/or impingement ring 55 may provide different fluid motion in comparison to other shapes. In some embodiments, the proximal chamber 60 and distal chamber 70 of the fluid platform 2 may include any other cross-sectional shape, including oval, tear-drop, polygonal, etc.

FIG. 24 is a side sectional view of a bottom cap 92 of a fluid platform 2 according to some embodiments. As described herein, in some embodiments, the bottom cap 92 can define a distal chamber 70. As shown, the bottom cap 92 may comprise a proximal opening 96 (e.g., a superior opening), a distal opening 97 (e.g., an inferior opening), and a transition 95 disposed between the proximal opening 96 and distal opening 97, all of which are in fluid communication with one another. The proximal opening, distal opening and transition 95 may define the distal chamber 70. As shown, the proximal opening 96 may be of a different (e.g., larger) cross-sectional area than a cross-sectional area of the distal opening 97, with the change in cross-sectional area occurring across the transition 95. In some embodiments, the transition 97 may taper between the proximal opening 96 and the distal opening 97. In some embodiments, the proximal opening 96 may be between 2 mm to 6 mm, between 3 mm and 5 mm, 4 mm, or about 4 mm and the distal opening 97 may be between 1 mm and 5 mm, between 2 mm and 4 mm, 3 mm or about 3 mm. In some embodiments, the bottom cap 92 may comprise an access opening 18 as described herein. In some embodiments, the bottom cap 92 may comprise a flange 16 as described herein. The embodiment of FIG. 24 can be coupled to a proximal chamber 60 (e.g., couple to a main body 40 having a proximal chamber 60) having a larger (e.g., 4 mm) transition opening 30 to allow for use of a larger proximal chamber for treatment of a tooth having a smaller access opening 18 (e.g., 3 mm).

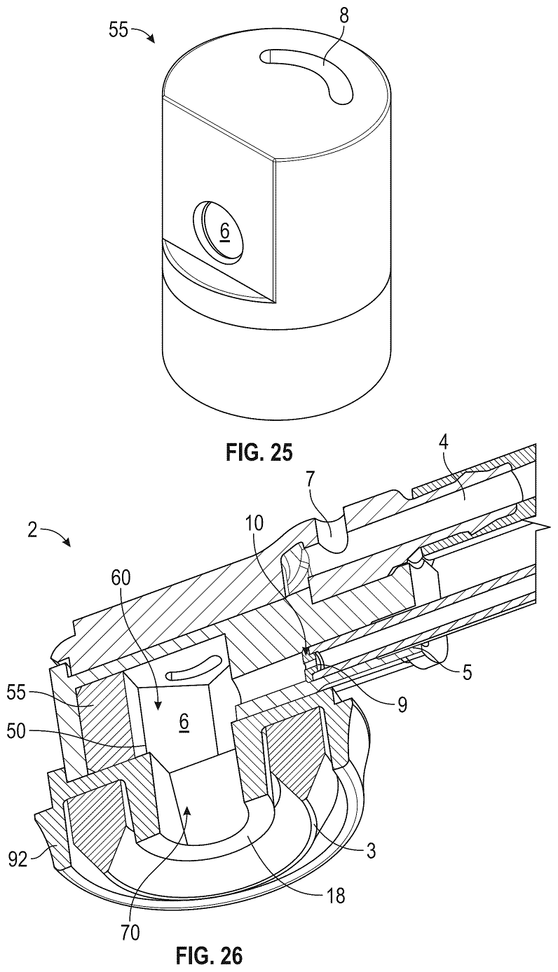

FIG. 25 is a top perspective view of an impingement ring 55 according to some embodiments. As shown, the impingement ring 55 may be a unitary piece including within a chamber 6 and a suction port 8. In some embodiments, the impingement ring 55 may be molded or machined as one unitary piece. In some embodiments, the chamber 6 within the impingement ring 55 may have an inner cross-sectional diameter between 2 mm to 6 mm, between 3 mm and 5 mm, 4 mm, or about 4 mm.

FIG. 26 is a bottom perspective sectional view of a fluid platform 2 according to some embodiments. As shown and as described herein, in some embodiments a chamber 6 the fluid platform 2 may have a polygonal cross-sectional shape and/or a cross-sectional shape that is not round and/or elliptical (e.g., a polygonal shape when a cross-section is taken along a plane formed by an anterior-posterior axis and a left-right axis as shown relative to FIG. 11F). In some embodiments, one or both of a proximal chamber 60 and a distal chamber 70 can have a polygonal cross-sectional shape and/or a cross-sectional shape that is not round or elliptical. Further, in some embodiments an impingement ring 55 may have a polygonal and/or non-round cross-sectional shape. For example, the inner walls of a proximal chamber 60, the inner walls of a distal chamber 70, and/or the inner surface of the impingement ring 55 may comprise polygonal segments including planar surfaces connected by edges. The inner cross-sectional shape of the proximal chamber 60, the impingement ring 55, and/or the distal chamber 60 may be square, hexagonal, or any other polygonal shape or include segments that are square, hexagonal, and/or any other polygonal shape. In some embodiments, the inner walls of the proximal chamber 60, the inner walls of the distal chamber 70, and/or the inner surface of the impingement ring 55 may have polygonal shapes that are different from one another.

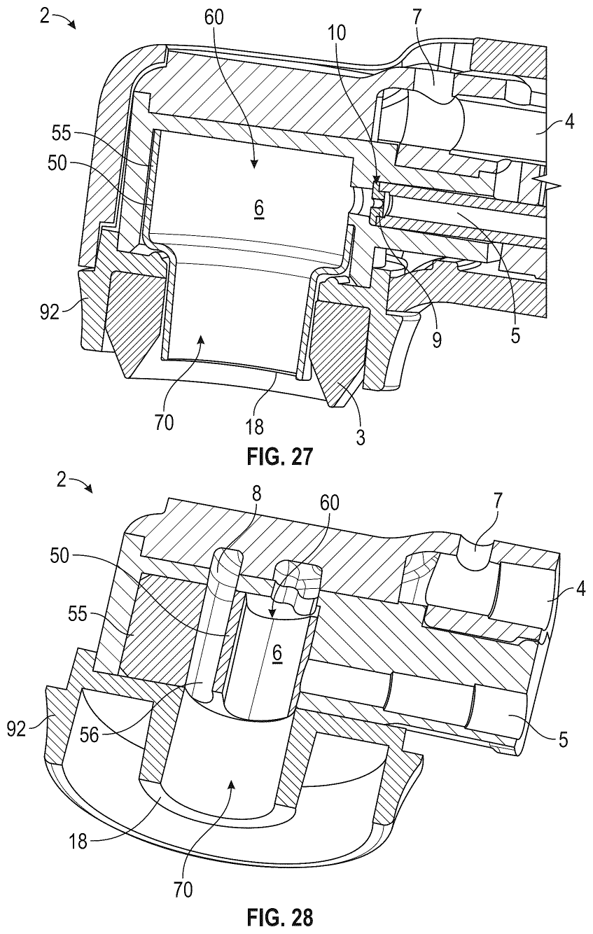

FIG. 27 is a top perspective sectional view of a fluid platform 2 according to some embodiments. As shown in FIG. 27, in some embodiments, an impingement ring 55 (or a sidewall of the chamber 6) may be formed as a continuous structure extending superiorly to inferiorly within the fluid platform 2 and forming a proximal chamber 60 and a distal chamber 70 in fluid communication with one another. As shown in FIG. 27, in some embodiments the impingement ring 55 (or a sidewall of the chamber 6) may comprise a variable cross-sectional area (e.g., for a cross-section taken along a plane formed by an anterior-posterior axis and a left-right axis as shown relative to FIG. 11F). In some embodiments, the impingement ring 55 (or a sidewall of the chamber 6) may comprise a larger cross-sectional area (e.g., for a cross-section taken along a plane formed by an anterior-posterior axis and a left-right axis as shown relative to FIG. 11F) for forming the proximal chamber 60 and a smaller cross-sectional area for forming the distal chamber 70. In some embodiments, the inferior end of the impingement ring 55 (or a sidewall of the chamber 6) forms the access port 18. Further as shown and in some embodiments, a sealing cap 3 may be disposed between an outer surface of the impingement ring 55 (or a sidewall of the chamber 6) and a portion of a bottom cap 92, with the side wall of the impingement ring 55 (or a sidewall of the chamber 6) forming an access port 18. In some embodiments, the impingement ring 50 (or a sidewall of the chamber 6) may include a tapered (and/or funnel shaped) region at the boundary between the proximal chamber 60 and the distal chamber 70. A transition opening (such as transition opening 30 described herein) can be positioned within the transition region.

FIG. 28 is a bottom perspective sectional view of a fluid platform 2 according to some embodiments. As shown in FIG. 28, in some embodiments, an impingement ring 55 of the fluid platform 2 may comprise an impingement member 50, which when inside a fluid platform 2 may be disposed over (e.g., superior to) a transition opening and/or an access port 18 of the fluid platform 2. In some embodiments, the impingement member 50 may be in the form of a partition extending over the transition opening and/or access port. In some embodiments, the impingement ring 55 may include a suction port or region 56 disposed anterior to the impingement member 50. The impingement member 50 may separate the proximal chamber 60 from the suction port 56. In some embodiments the suction port 56 can be in fluid communication with a suction port 8 of the fluid platform 2 at one end (e.g., superiorly as shown) and in fluid communication with a distal chamber 70 of the fluid platform 2 at the other end (e.g., inferiorly as shown). In this configuration, fluid evacuation may be split from the proximal chamber 60 and occur closer to a tooth 110 and its treatment region. In some embodiments, the impingement ring 55 may be as described relative to the impingement ring 55 of FIG. 17.

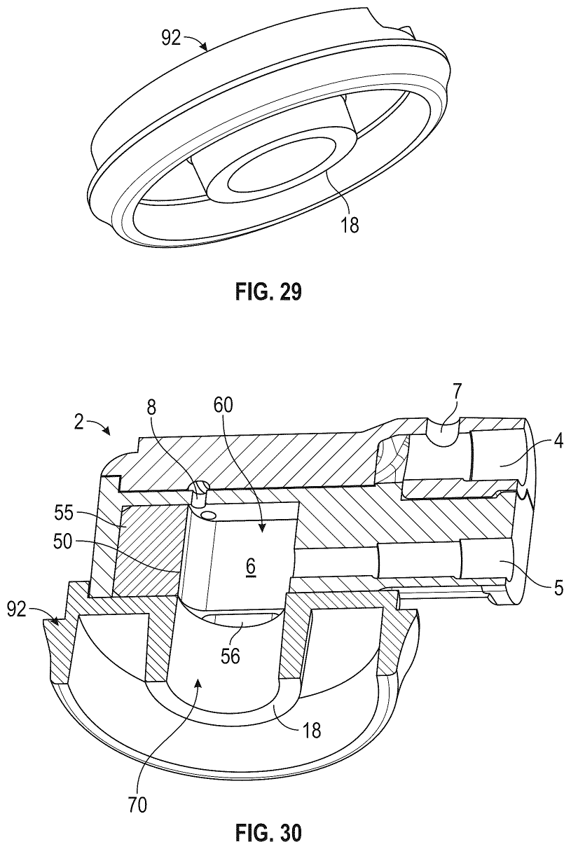

FIG. 29 is a bottom perspective view of a bottom cap 92 according to some embodiments. As shown, in some embodiments the bottom cap 92 may comprise a relatively compact structure in an inferior-superior axis and be compatible with a sealing cap 3 (not shown) of similarly compact proportions. In this configuration and when coupled to a fluid platform 2 as described herein, the more compact structure of the bottom cap 92 may allow for a fluid jet 20 produced by the fluid platform 2 to be in closer proximity to a treatment region of a tooth 110.

FIG. 30 is a bottom perspective sectional view of a fluid platform 2 according to some embodiments. As shown, in some embodiments an impingement ring 55 of the fluid platform 2 may comprise suction regions or ports 56 disposed at its right side and/or its left side (left side not shown in this cross-sectional view) in fluid communication with a suction port 8 of the fluid platform 2 at one end (e.g., superiorly, not shown in this cross-sectional view) and with a distal chamber 70 of the fluid platform 2 at the other end (e.g., inferiorly). Further as shown, in some embodiments the proximal chamber 60 may comprise one or more additional suction ports 8 disposed at a superior (and, in some embodiments, anterior) inner wall of the proximal chamber 60. Anterior ports 8 within the proximal chamber 60 may assist in creating lower apical pressures. The suction ports 56 can be separated from the chamber 60 by partitions or walls and can have inferior ends positioned to draw waste or effluent fluid from the distal chamber 70. Combined, in this configuration fluid flow in a proximal chamber 60 of the fluid platform 2 may be at least partially separated from the flow of waste or effluent fluid.

FIG. 31 is a bottom perspective sectional view of a fluid platform 2 according to some embodiments. As shown in FIG. 31, in some embodiments a central axis of an inlet line 5 of the fluid platform 2 may be at an angle relative to an anterior-posterior axis of the fluid platform 2 (e.g., inclined superiorly), thus creating a fluid jet 20 in use that may travel across a proximal chamber 60 at an angle relative to an anterior-posterior axis of the fluid platform 2 (e.g., inclined superiorly). Similar to FIG. 30, the fluid platform 2 as shown in FIG. 21 may include an impingement ring 55 with side suction ports 56, and suction port(s) 8 disposed at a superior and anterior inner wall of the proximal chamber 60. In some embodiments, similar to FIG. 29, the fluid platform 2 can include a relatively compact bottom cap 92.

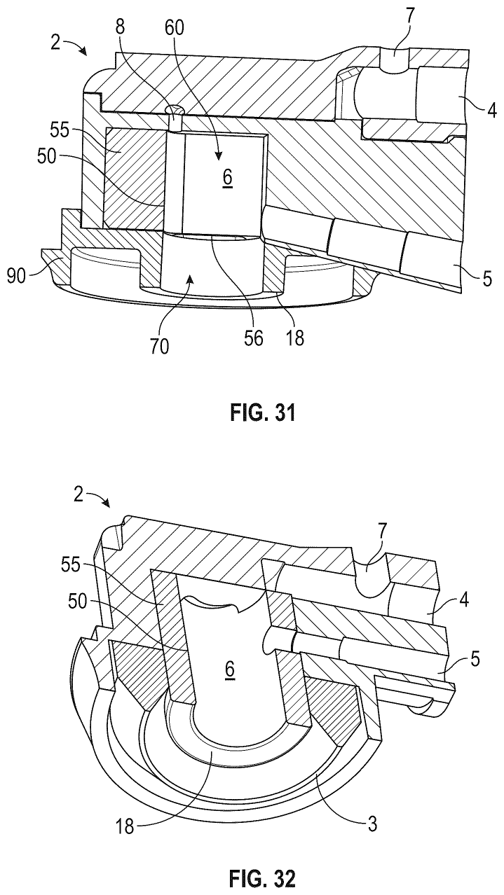

FIG. 32 is a bottom perspective sectional view of a fluid platform 2 according to some embodiments. A shown in FIG. 32, in some embodiments, the fluid platform 2 may include a unitary molded body with an impingement ring 55 having an impingement member 50 disposed within the unitary molded body. In some embodiments, the impingement ring 55 may be machined and or formed from a thick wall tube and may extend superiorly to inferiorly across the fluid platform 2 to form a chamber 6. In some embodiments, an inferior end of the impingement ring 55 can form an access port 18. In some embodiments, the unitary molded body of the fluid platform 2 may be molded over the impingement ring 55. In some embodiments, the molded body of the fluid platform 2 may comprise a sealing cap 3 as described herein or a compact sealing cap 3.

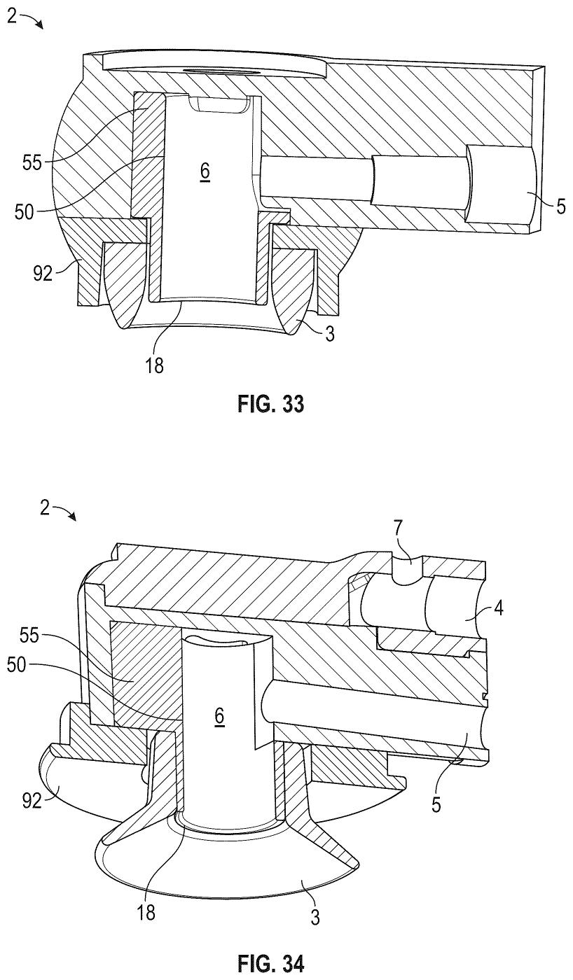

FIG. 33 is a bottom perspective sectional view of a fluid platform 2 according to some embodiments. In some embodiments the fluid platform 2 may comprise a substantially spherical outer surface. In some embodiments, the fluid platform 2 is configured to swivel relative to a handpiece 12 of a treatment instrument 1. The fluid platform can be configured to align to a treatment area (and/or a platform 405 as described herein) independent of the position of the handpiece 12. In this configuration, an o-ring seal may be utilized to form a seal at inlet line 5 and accommodate any movement of the fluid platform 2 relative to the handpiece 12.

FIG. 34 is a bottom perspective sectional view of a fluid platform 2 according to some embodiments. As shown, in some embodiments the sealing cap 3 of the fluid platform 2 may be in the form of a suction cup. For example, the sealing cap can have an outward flaring cone-like shape. In this configuration, the sealing cap 3 may accommodate any misalignment between the fluid platform 2 and a treatment area (and/or a platform 405 as described herein).

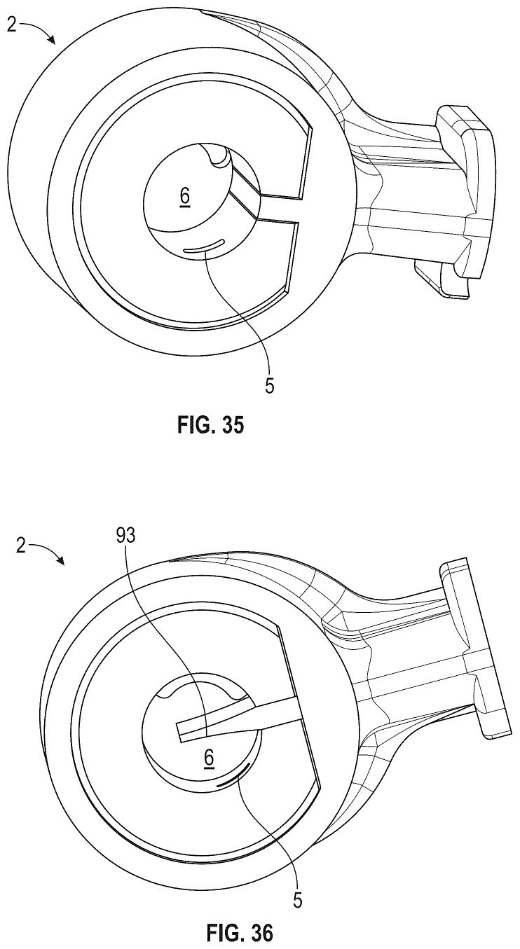

FIG. 35 is a bottom perspective sectional view of a fluid platform 2 according to some embodiments. As shown in FIG. 35, in some embodiments a central axis of an inlet line 5 may open into a chamber 6 of the fluid platform 2 offset from an anterior-posterior axis of the fluid platform 2. In some embodiments, the central axis of the inlet line 5 may be tangential with the chamber 6. In some embodiments, the central axis of the inlet line 5 may be positioned at an angle relative to the anterior-posterior axis, an inferior-superior axis, and/or a left-right axis of the fluid platform 2.

FIG. 36 is a bottom perspective sectional view of a fluid platform 2 according to some embodiments. As shown in FIG. 36, in some embodiments a central axis of an inlet line 5 may open into a chamber 6 of the fluid platform 2 offset from an anterior-posterior axis of the fluid platform 2. In some embodiments, the central axis of the inlet line 5 may be tangential with the chamber 6. In some embodiments, the central axis of the inlet line 5 may be positioned at an angle relative to the anterior-posterior axis, an inferior-superior axis, and/or a left-right axis of the fluid platform 2. In some embodiments the fluid platform 2 may comprise a clapper 93. The clapper 93 can extend from a posterior side of the chamber 6 at least partially across the center of the chamber 6 (e.g., along a plane formed by the inferior-superior and anterior-posterior axes). The clapper 93 can be configured to modify fluid motion within the chamber 6.

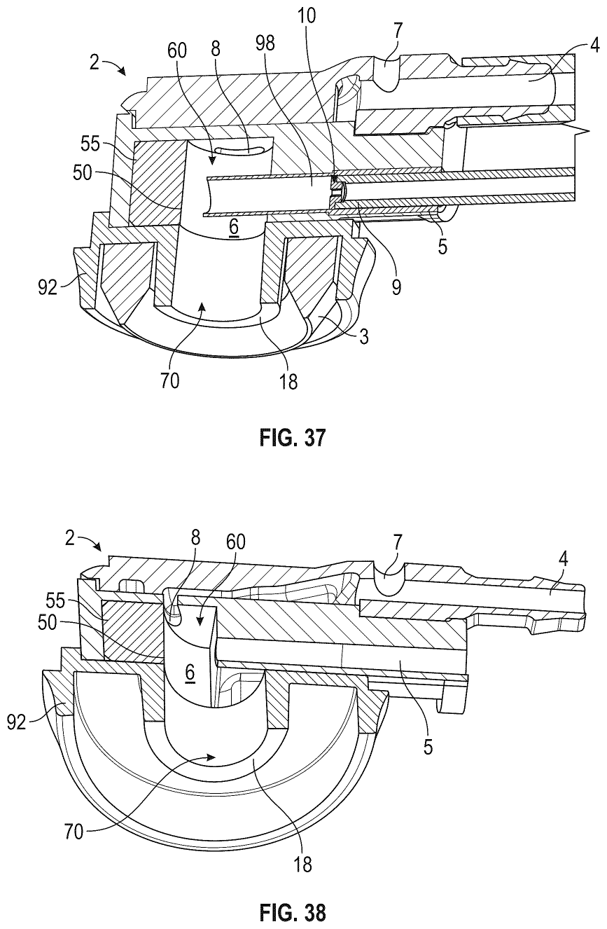

FIG. 37 is a bottom perspective sectional view of a fluid platform 2 according to some embodiments. As shown in FIG. 37, in some embodiments the fluid platform 2 may comprise a channel or tunnel 98 fluidly connected to and extending from an inlet line 5 into a proximal chamber 60 of the fluid platform 2. In some embodiments, the channel 98 can extend along an axis coextensive with a jet axis of a jet produced by a nozzle 9. As shown in FIG. 37, the channel 98 may shield at least a portion of the fluid jet 20 produced by the nozzle 9 until the fluid jet 20 impinges upon an impingement member 50. In some embodiments, a suction port 8 of the fluid platform 2 may be separated from at least a portion of the fluid jet 20 by the channel 98.

FIG. 38 is a bottom perspective sectional view of a fluid platform 2 according to some embodiments. As shown, in some embodiments an inlet line 5 of the fluid platform 2 may extend into a proximal chamber 60 of the fluid platform 2 beyond an inner surface of a distal chamber 70 of the fluid platform 2 (e.g., in relation to a superior-inferior axis of the fluid platform 2). In some embodiments, the inlet line 5 may extend at least partially over a transition opening between the proximal chamber 60 and the distal chamber 70. In some embodiments, the fluid platform 2 may comprise one or more suction ports 8 disposed at a superior inner wall of the proximal chamber 60 at a position anterior to the anterior end of the inlet line 5.

Examples of Matrices for Use with Treatment Instruments

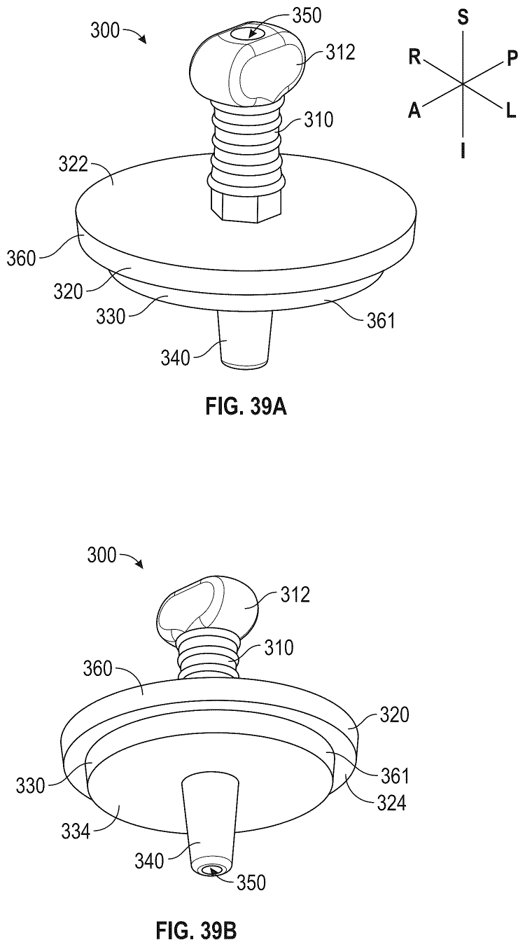

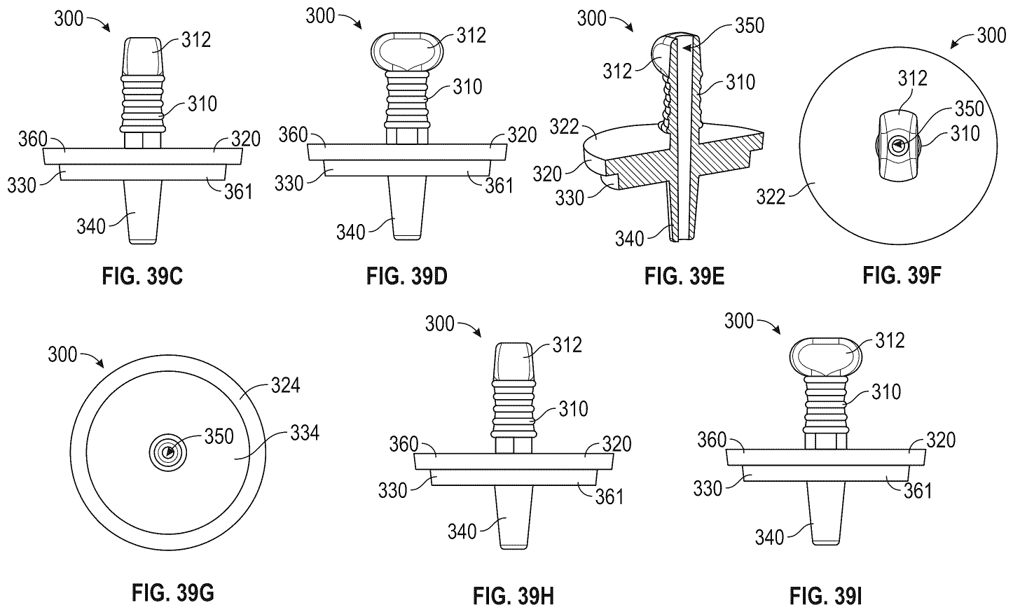

FIGS. 39A-411 disclose various embodiments related to a matrix 300. The matrix 300 can be used in conjunction with a sealant material or conforming material 400 as described herein to facilitate the cleaning and/or filling of a treatment region of a tooth 110. In some embodiments, the matrix 300 can be an applicator used to apply the conforming material 400 to a tooth to form a platform 405 on the tooth, as described in further detail herein. In some embodiments, the matrix 300 be a frame, scaffolding, or mold for formation of the platform 405 from the conforming material 400. In some embodiments, the matrix 300 can be used to form the platform 405 of conforming material 400 on the tooth without requiring a tooth cap or other hardware to be attached to the tooth. The platform 405 can be used to support a treatment instrument (e.g., to support a fluid platform 2 of a treatment instrument 1) during a treatment procedure.

FIG. 39A includes three-dimensional coordinate axes indicating superior (S), inferior (I), anterior (A), posterior (P), left (L), and right (R) directions. As shown in FIG. 39A, the right direction R is generally pointing into the page and the left direction L is generally pointing out of the page. These directions are provided for reference only to provide examples of relative positions of aspects of a matrix 300 and may not reflect the particular anatomical positions of the matrix 300 when in use.

FIG. 39A is a top perspective view and FIG. 39B is a bottom perspective view of a matrix 300 according to some embodiments. In some embodiments, the matrix 300 may include a handle 310, an upper rim or ledge 320, a lower rim or ledge 330, and a pin 340.

In some embodiments, the handle 310 can include a handle top 312. The handle top 312 may be disposed at a superior end of the handle 310. The handle 310 can be in the form of a generally longitudinal structure extending along the superior-inferior axis. In some embodiments, an inferior end of the handle 310 may connect to an upper surface 322 of the upper rim 320 at a center of the upper surface 322.

In some embodiments, the upper rim 320 can include the upper surface 322 and a lower surface 324. The upper rim 320 can be positioned below (inferior to or distal to) the handle 310. In some embodiments, the upper rim 320 may be disc shaped or generally disc shaped. The upper rim 320 may have a circular cross-section in a plane formed by the right-left and anterior-posterior plane and have a height or thickness along the superior-inferior axis.

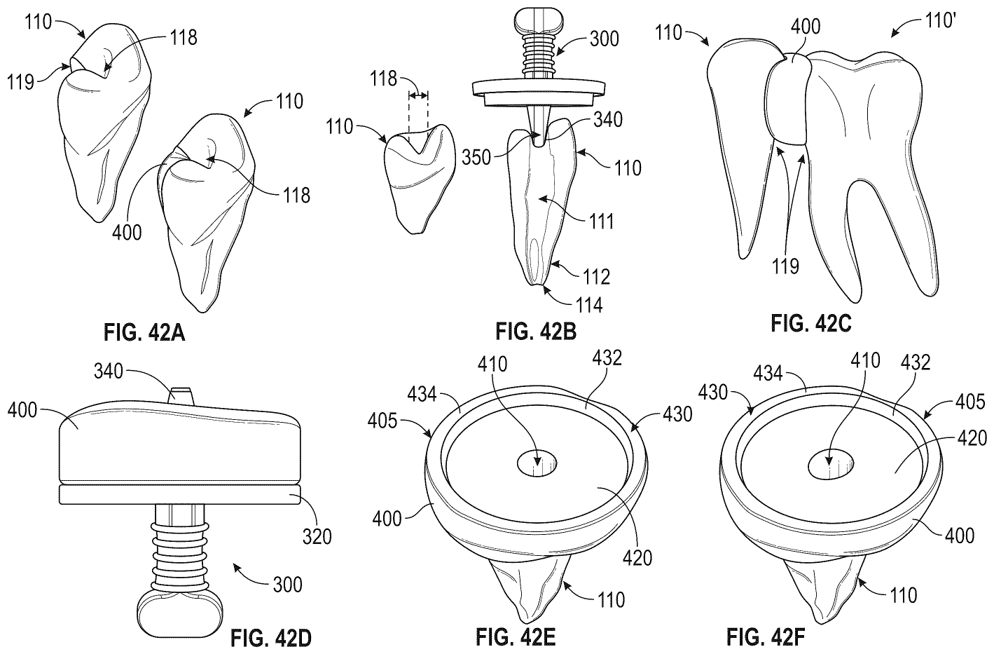

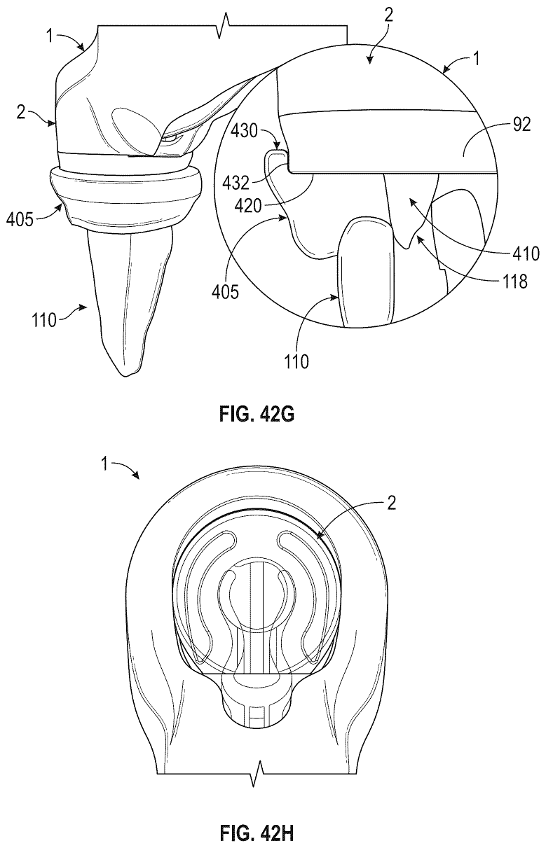

In some embodiments, the lower rim 330 can include a lower surface 334. The lower rim 330 can be positioned below (inferior to or distal to) the upper rim 320. The lower rim 330 can be disc shaped or generally disc shaped. The lower rim 330 may have a circular cross-section in a plane formed by the right-left and anterior-posterior plane and have a height or thickness along the superior-inferior axis. In some embodiments, the lower rim 330 can be concentric with the upper rim 320. As shown in FIGS. 39A and 39B, in some embodiments, the lower rim 330 may have a smaller width (e.g., a smaller cross-section or smaller diameter) than the upper rim 320. For example, an outer edge 360 of the upper rim 320 can extend beyond an outer edge 361 of the lower rim 330. The lower rim 330 may connect at its superior end to the lower surface 324 of the upper rim 320. In some embodiments, the lower rim 330 and upper rim 320 can be used to form a platform 405 from the conformable material 400. As described further herein, the shapes of the lower rim 330 and upper rim 320 can form corresponding shapes of the platform 405. For example, in some embodiments, a conforming material 400 can be applied to the matrix 300 over the lower surface 324 of the upper rim 320 and the lower surface 334 of the lower rim 330 and can adopt a corresponding shape. An example of conforming material 400 applied to the matrix 300 is shown in FIG. 42D. The matrix 300 can then be used to apply the shaped conforming material 400 to a tooth to form the platform 405, as described in further detail herein, for example, as shown in FIG. 42E.

The pin 340 may extend inferiorly (distally) from the lower rim 330. In some embodiments, the pin 340 can be in the form of a generally longitudinal structure extending along the superior-inferior axis. In certain embodiments, the pin 340 may form an access opening having a corresponding shape within the platform 405. The access opening can allow a portion of a treatment instrument to access a treatment region of the tooth. The access opening can allow fluid communication between the treatment instrument and the treatment region of the tooth. In some embodiments the pin 340 may taper in the inferior (distal) direction. In some embodiments, the pin 340 can have a tapered shape to facilitate removal from the platform 405 after the platform 405 is formed.

As shown in FIG. 39E, in some embodiments, the matrix 300 can include a channel 350 in the form of a through hole that extends through the matrix from a superior end to an inferior end (e.g., along the superior-inferior axis or central axis of the matrix 300). In some embodiments the channel 350 may have a constant cross-sectional area. In some embodiments the channel 350 may have a variable cross-sectional area, for example, with a cross-sectional area that increases in the superior direction. In some embodiments, the channel 350 may act as a vent channel or relief channel to prevent the buildup of pressure within the tooth during formation of the platform 405, as discussed in further detail herein.

In some embodiments and as shown in FIGS. 39A-39B, the handle top 312 may be elongated along the anterior-posterior axis. In some embodiments, an elongated handle top 312 may facilitate handling of the matrix 300 by a clinician. In some embodiments, the handle 310 may comprise circumferential ridges and/or other protuberances to facilitate grasping of the handle 310 by a clinician.

FIG. 39C is a front view, FIG. 39D is a side view, FIG. 39F is a top view, FIG. 39G is a bottom view, FIG. 39H is a rear view, and FIG. 39I is a second side view showing the opposite side of FIG. 39D of the matrix 300 shown in FIGS. 39A-39B. As shown in FIGS. 39C-39D and 39H-39I, in some embodiments, the outer edge 360 and the outer edge 361 may comprise radially outward facing surfaces that taper in the inferior direction. A taper in the outer edge 360 of the upper rim 320 and the outer edge 361 of the lower rim 330 may facilitate removal of the matrix 300 after being used to create the platform 405 out of the conformable material 400 as described further herein. As shown in FIGS. 39C-39D and FIGS. 39F-39G, the elements comprising the matrix 300 may share a common superior-inferior central axis.

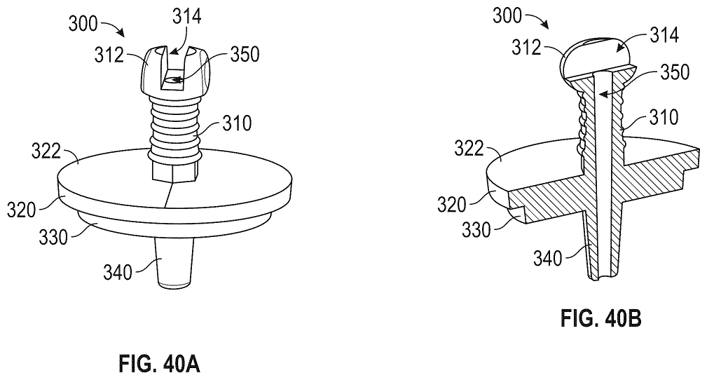

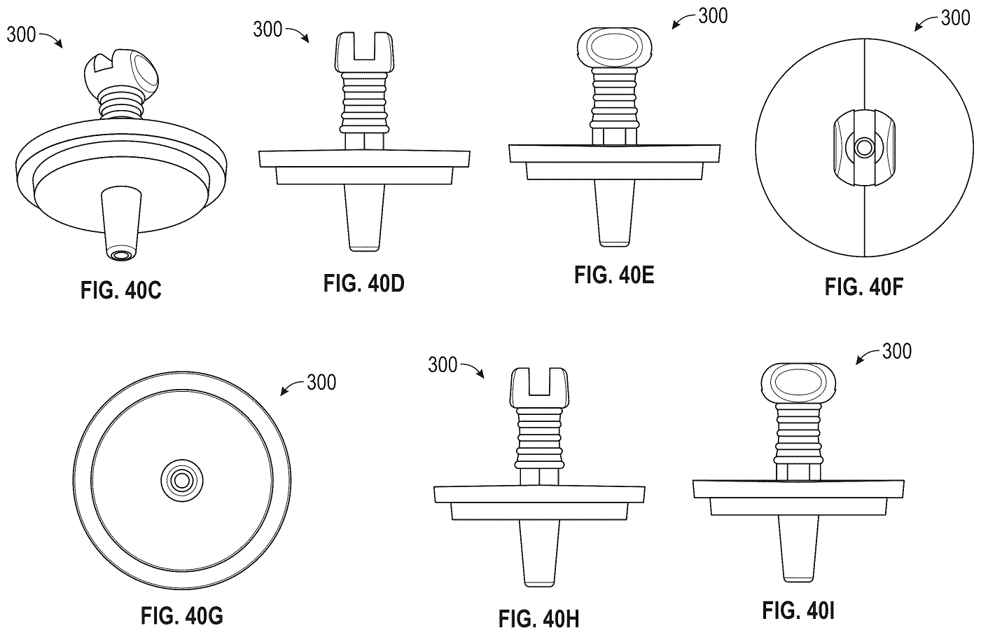

FIG. 40A is a top perspective view and FIG. 40B is a top perspective sectional view of a matrix 300 according to some embodiments. FIG. 40C is a bottom perspective view, FIG. 40D is a front view, FIG. 40E is a side view, FIG. 40F is a top view, FIG. 40G is a bottom view, FIG. 40H is a rear review, and FIG. 40I is a second side view showing the opposite side of FIG. 40E of the matrix 300 of FIG. 40A. As shown, in some embodiments the matrix 300 may comprise a recess 314 in the handle top 312. The recess 314 may extend inferiorly from a superior surface of the handle top 312 and across the width of the handle top 312. The recess 314 can extend transverse to (e.g., perpendicular to) the central axis of the matrix 300 or central axis of the channel 350. In some embodiments, the recess 314 may fluidly connect to the channel 350 as shown in FIG. 40A. In some embodiments, the recess 314 may act as a vent to prevent the buildup of pressure in the tooth during formation of the platform 405. For example, the recess 314 may allow for venting in a lateral direction relative to the central axis of the matrix. For example, if a top surface of the handle top 312 is blocked when the handle 310 is grasped by a clinician, preventing venting in the superior direction, air can flow through the recess 314 to reduce pressure in the tooth. In some embodiments, the recess 314 may form a continuous fluidic connection between atmosphere and the channel 350 even when the handle 310 is grasped by a clinician.

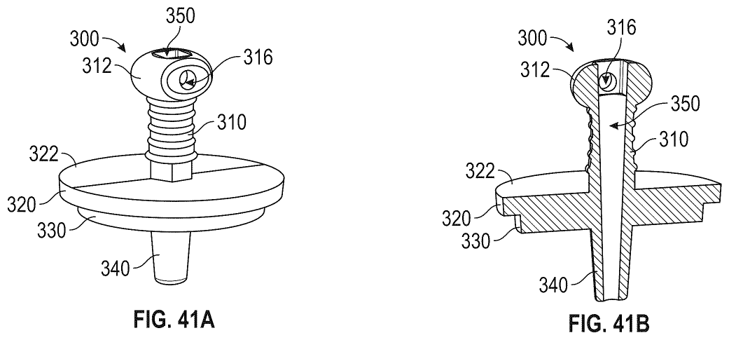

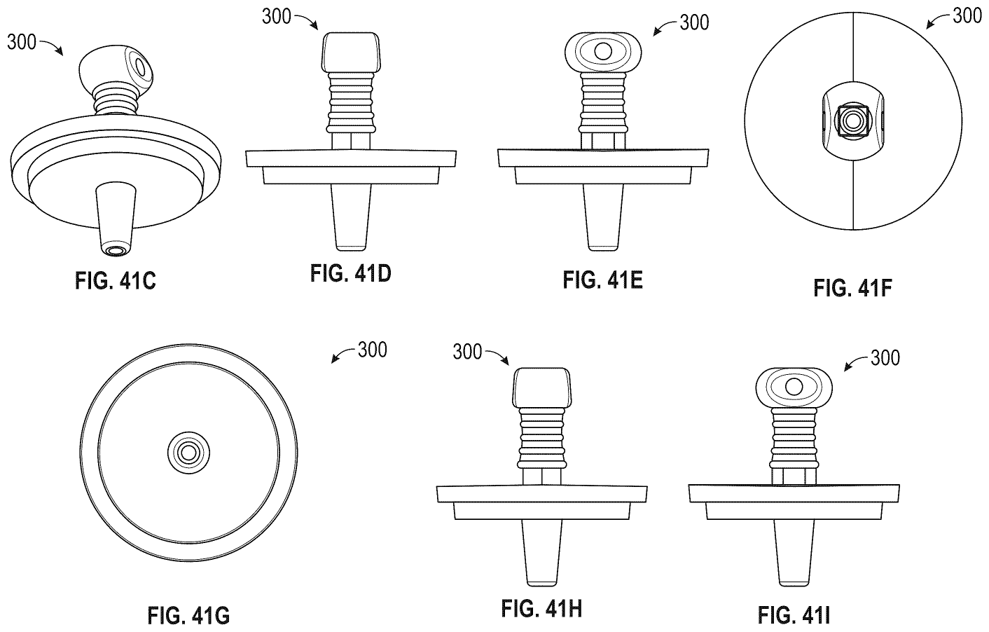

FIG. 41A is a top perspective view and FIG. 41B is a top perspective sectional view of a matrix 300 according to some embodiments. FIG. 41C is a bottom perspective view, FIG. 41D is a front view, FIG. 41E is a side view, FIG. 41F is a top view, FIG. 41G is a bottom view, FIG. 41H is a rear review, and FIG. 41I is a second side view showing the opposite side of FIG. 41E of the matrix 300 of FIG. 41A.

As shown in FIGS. 41A-B, in some embodiments the matrix 300 may include a channel 316 extending through the handle top 312. The channel 316 may extend through a width of the handle top 312 in the form of a through hole. In some embodiments, the handle top 312 can intersect with the superior-inferior central axis of the matrix 300. In some embodiments, the channel 316 may extend transverse to (e.g., perpendicular to) the central axis of the matrix 300 or central axis of the channel 350. In some embodiments, the channel 316 may be disposed at a geometric center of the handle top 312 when viewed from its side (e.g., with the left-right axis in-line with the line of sight). In some embodiments, the channel 316 may fluidly connect to the channel 350 of the matrix 300. In some embodiments, the channel 316 can extend across a center of the channel 350. In some embodiments, the channel 316 can act as a vent to prevent the buildup of pressure in the tooth during formation of the platform 405. For example, the channel 316 may allow for venting in a lateral direction relative to the central axis of the matrix. For example, if the superior end of the channel 350 at the top surface of the handle top 312 is blocked when the handle 310 is grasped by a clinician, preventing venting in the superior direction, air can flow through the channel 316 to reduce pressure in the tooth. In some embodiments, the channel 316 may form a continuous fluidic connection between atmosphere and the channel 350 even when the handle 310 is grasped by a clinician. In some embodiments, the channel 316 can be shaped, dimensioned, and/or otherwise configured to receive a tool for use in removing the matrix 300 after formation of the platform 405.

### Example Process of Treating a Tooth Using a Treatment Instrument, a Fluid Platform, a Matrix, and a Sealant

FIGS. 42A-42H disclose various aspects of a process for treating a tooth 110. As described below, in some embodiments, the process includes formation of a platform 405 using a matrix 300 and treatment of the tooth using a treatment instrument 1 after formation of the platform 405.

With reference to FIG. 42A, in some embodiments, a clinician may remove caries and defective restorations from a tooth 110. In some embodiments, the clinician may restore missing tooth structure. For example, the clinician may use a sealant material or conforming material 400 to temporarily restore the tooth structure (e.g., the exterior surface 119 of tooth 110 in FIG. 42A). The conforming material 400 may comprise a conforming light-cure resin, such as the SoundSeal® conforming light-cure resin from Sonendo®.

With reference to FIG. 42B, the clinician may prepare an endodontic access opening 118. In some embodiments, the physician can prepare the access opening 118 to allow unrestricted conservative straight-line access to the tooth 110. For example, the clinician may form the endodontic access opening 118 to a minimum opening size (e.g., diameter) per standard endodontic practice. For example, if treating a premolar, the clinician may create an endodontic access opening 118 with a minimum diameter of 1.5 mm. As another example, if treating a molar, the clinician may create an endodontic access opening 118 with a minimum diameter of between 2.7 mm to 3.0 mm. In some embodiments, as shown in FIG. 42B, after formation of the endodontic access opening 118, the clinician may test fit a matrix 300 of an appropriate size as described herein with the endodontic access opening 118. For example, if treating a premolar, the clinician may test that the first 2 mm of the pin 340 of the matrix 300 can be inserted into the endodontic access opening 118. As another example, if treating a molar, the clinician may test that the entire pin 340 of the matrix 300 can be inserted into the endodontic access opening 118. In either example, the clinician may ensure that no interference exists between a matrix 300 and patient anatomy or a dental dam clamp if present. The clinician may also locate each canal orifice within the root 112 of tooth 110 and ensure removal of all pulp stones or other obstruction(s) to each canal to ensure an unobstructed fluid pathway exists from the endodontic access opening 118 through the pulp cavity 111 to the apex 114.

The clinician may estimate the canal length using an apex locator, going to the mark ‘Apex’ (full tone) and note the length, or by using a pre-op CBCT. The procedure working length of the system 100 may be set to 1.0 mm short of the canal length measurement. For teeth with special anatomies, the working length of the system 100 may be set to 2.0 mm short of the canal length measurement. If the treatment procedure is a retreatment, in some embodiments, a clinician may insure obturation material and/or solvent are removed, and may use a larger instrument size.

With reference to FIG. 42C, the clinician may clean the entire tooth 110 and, in some embodiments, may in addition clean any neighboring teeth 110′, including their occlusal surfaces. The clinician may use isopropyl alcohol for the cleaning and then air dry the teeth. In some embodiments, the clinician may inject the conforming material 400 into the interproximal surfaces of the teeth (e.g., between the exterior surfaces 119) and fully cure the sealant.

With reference to FIGS. 42D-42F, a sub-process for forming a platform 405 is described. In some embodiments, the clinician may apply (e.g., inject) the conforming material 400 onto an overturned matrix 300. As shown in FIG. 42D, the conforming material can be applied up to the upper rim 320, covering the lower surface 334 of the lower rim 330, the outward radial edge 361 of the lower rim 330, the lower surface 324 of the upper rim, and extending around a portion of the pin 340. The conforming material 400 can be applied so that a distal end of the pin 340 extends distally beyond the conforming material 400. The clinician may then place the matrix 300 with sealant 400 in an uncured state on the tooth 110 with the pin 340 inserted into the endodontic access opening 118. For example, for a premolar, the clinician may place the matrix 300 ensuring that the first 2 mm of the pin 340 can be inserted into the endodontic access cavity 118. As another example, for a molar, the clinician may place the matrix 300 ensuring that the lower surface 334 of the lower rim 330 contacts the highest cusps(s)/occlusal surface of the tooth 110 and the lower surface 334 remains substantially parallel to a floor of the pulp cavity 111 and substantially perpendicular to the walls of the pulp cavity 111. With the matrix 300 with sealant 400 in place, the clinician may then cure the conforming material 400 (e.g., by light curing) until the conforming material is fully cured. In embodiments of the matrix 300 with a channel 350, the channel 350 may serve as a relief channel for air and prevent the formation of voids within the conforming material 400 while curing. In some procedures, in the absence of vent pathways from the tooth, the application of a platform 405 may cause an increase in pressure within the tooth that creates voids within the conforming material 400. The channel 350 and/or recess 314 or channel 316 can allow for the release of pressure from the tooth without the formation of voids in the conforming material.

FIG. 42E shows the platform 405 on the tooth 110 after curing as formed by the matrix 300. As shown, the platform 405 may include features that correspond to or substantially mirror (e.g., form a negative of) the features of the matrix 300, such as a surface 420 that corresponds to the lower surface 334 of the matrix 300, a ridge wall 432 that corresponds to the outer edge 361 of the lower rim 330, a ridge surface 434 that corresponds to the lower surface 324 of the matrix 300, and an access opening 410 that corresponds to the exterior shape of a portion of the pin 340 of the matrix 300. The ridge wall 432 and the ridge surface 434 of the platform 405 may together comprise a ridge 430, with the ridge surface 434 being offset from the surface 420 of the platform 405 (e.g., raised above the surface 420 as oriented in FIG. 40E). Due to the positioning of the pin 340 within the endodontic access opening 118 during curing of the platform 405, the access opening 410 of the platform 405 may be in fluid communication with the endodontic access opening 118 of the tooth 110 as shown in FIG. 42E. In some embodiments, after removal of the matrix 300, the clinician may reaccess the access opening 410 by reforming the access opening 410 of the platform (e.g., by removing cured sealant) to increase the size of the access opening 410 and/or change the shape of the access opening 410 (e.g., to substantially match and/or form a smooth transition with the endodontic access opening 118). An example of a reaccessed access opening 410 is shown in FIG. 42F.

With reference to FIG. 42G, after the platform 405 has been formed on the tooth 110, the platform 405 can receive a treatment instrument, such as treatment instrument 1. For example, a fluid platform 2 of the treatment instrument 1 may be positioned on the surface 420 of the platform 405 within ridge 430. The ridge 430 may assist in locating the fluid platform 2 at the center of the platform. The ridge 42 may also restrict or prevent movement of the treatment instrument along the surface 420 of the platform (e.g., left-right and anterior posterior movement). For example, the ridge 430 may prevent movement by more than 0.010 in.

In some embodiments, as shown in the inset of FIG. 42G, a bottom cap 92 of the fluid platform 2 may be placed within ridge 430 and adjacent ridge wall 432 of the platform 405. In some embodiments, the fluid platform 2 may comprise transparent and/or semi-transparent materials such that the clinician may see through at least part of the fluid platform 2 and visually align the fluid platform 2 with the endodontic access opening 118. In some embodiments, the clinician may place the fluid platform 2 centered to the platform 405 and substantially flat against the surface 420 of the platform 405. With this alignment between the fluid platform 2 and the platform 405, the clinician may gently press the fluid platform 2 against the platform 405 until fully engaged with the platform 405. In some embodiments (not shown), during engagement a sealing cap 3 may form a seal with the surface 420 of the platform 405, and the access port 18 of the fluid platform 2 may be fluidically coupled to the access opening 410 of the platform 405, the endodontic access opening 118 of the tooth 110, and the treatment area of the tooth 110.

With the engagement between the fluid platform 2 and the platform 405, the clinician may begin the procedure. The clinician may ensure any conduits 104 and/or tubing is not kinked or restricted. The clinician may ready a console 102 of the system 100 and press down on a foot pedal of the console, which may control the delivery of procedure fluid. The procedure may be paused by releasing the foot pedal. While pressing down on the console's foot pedal, the clinician may ensure that the fluid platform 2 remains properly seated on the platform 405 to retain the fluidic seal between the fluid platform 2, the access opening 410 of the platform 405, the endodontic access opening 118, and thus the treatment area of the tooth 110.

With reference to FIG. 42H, in some embodiments the clinician may monitor the procedure by visual and/or auditory cues indicating a seal has been created, or conversely, that a seal has been lost. In some embodiments, the fluid platform 2 may include one or more transparent or semi-transparent windows or sections. For example, at least a portion of the fluid platform 2 may be formed of a transparent or semi-transparent material. For example, in some embodiments, as shown in FIG. 42H, a superior surface of the fluid platform 2 can include a transparent or semi-transparent window. In some embodiments, the clinician may monitor the transparent or semi-transparent windows or sections for the presence of bubbles. In some embodiments, a fluid platform 2 free of bubbles may indicate a good seal. Conversely, in some embodiments, a fluid platform 2 with streaming bubbles may indicate a loss of seal. For auditory cues, there may be a distinct auditory change between a seal and loss of seal, which may correlate to the visual cues described above. If a seal is lost during the procedure, the clinician may attempt to regain the seal by slightly adjusting their hand position and thus the position of the fluid platform 2 against the platform 405. After an adjustment has been made, the clinician may wait for 1-3 seconds to allow any bubbles to clear and the auditory tone to change, indicating a good seal. The clinician may complete the procedure and proceed with standard endodontic post-cleaning procedures, including removal of the platform 405.

Although the example process as described relative to FIGS. 42A-42H has been given for a root canal procedure, the process may readily be adapted for the treatment of tooth surface caries or other tooth surface defects as described herein.

Example Process of Treating Dental Caries with a Treatment Instrument

As shown in FIG. 1B, various embodiments disclosed herein can be utilized in a treatment procedure to remove carious region(s) from an exterior surface of the tooth. In some embodiments, the treatment procedures can be used to remineralize and/or seal the treated region. Any of the treatment instruments 1 (and associated components and systems) disclosed herein (e.g., the treatment instruments 1 disclosed in FIGS. 1B-38) can be used to treat dental caries on exterior surfaces of a tooth.

Enamel is an outside layer of a healthy tooth, 96% of which is hydroxyapatite, a calcium compound. Dental caries can damage a tooth when acid produced by bacteria of an oral cavity accumulates such that topical pH on the tooth is low enough to demineralize the tooth structure. The loss of surface structure due to demineralization makes it easier for subsequent deposition and accumulation of substances inside the oral cavity, such as saliva, food residue, and bacteria, etc., initiating a cycle of tooth decay if no action is taken for treatment.

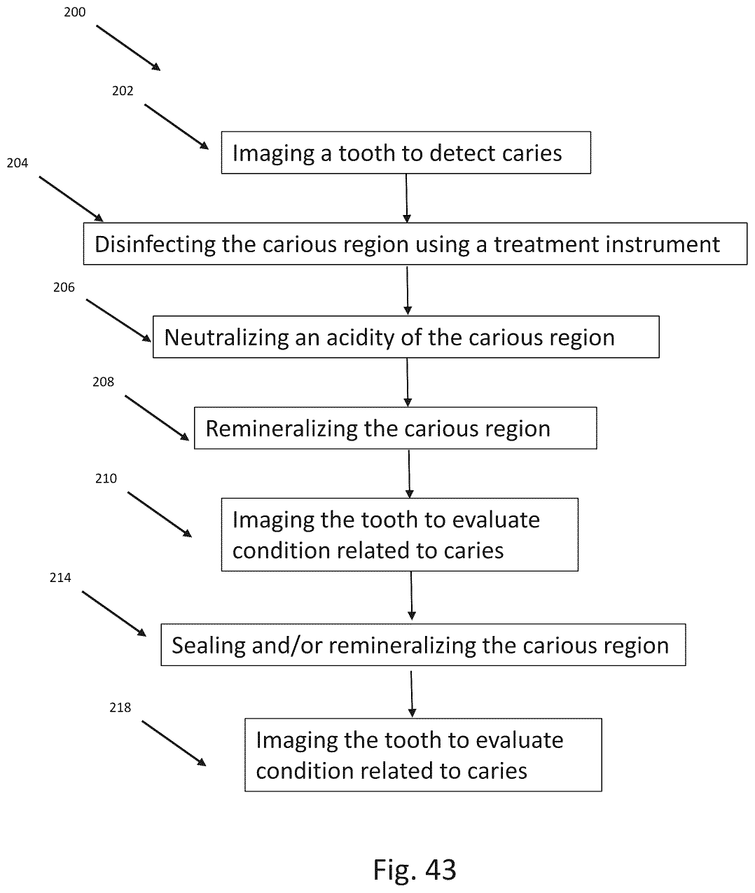

Various aspects of this disclosure includes a process or method of treating dental caries using a treatment instrument 1 in accordance with various embodiments. An exemplary process for treating dental caries can include using a treatment instrument 1, for example, a pressure wave generator 10, to clean tooth surfaces to remove extraneous residue such as biofilm (plaque), bacteria, food residue, and etc. Furthermore, actions such as sealing or direct remineralization can be taken to preserve the tooth or reconstruct the tooth decay to a healthier state or to its original health state. The method of treating dental caries according to this disclosure is described herein. Although the examples of FIGS. 43-50H are described in the context of treating carious regions on an exterior surface of the tooth, it should be appreciated that the systems and methods of FIGS. 43-50H can additionally be used in endodontic treatments to disinfect and neutralize diseased root canals.

FIG. 43 shows an exemplary process 200 of treating dental caries (e.g., cavitated and/or incipient caries). In some embodiments, the process of treating dental caries disclosed herein can include cleaning an infected tooth. In some embodiments, cleaning the infected tooth includes disinfecting the tooth. In various embodiments, the treatment process can be initiated if a carious region is detected on an external surface of the tooth.

Inside dental caries, as well as on exterior surfaces of an infected tooth, there usually exist extraneous substances, including but not limited to, food residue, deteriorated saliva, and/or active/inactive biofilm (e.g., plaque). Typically, cavitated lesions result in high levels of extraneous substances or bacteria which can lead to demineralization progressing into the dentin. The bacteria located on or within the cavitated lesion can invade the dentin and lead to tissue infection, if left untreated. Furthermore, incipient caries may also have a biofilm or bacteria present on the tooth which can lead to increased tooth acidity (e.g., due to the presence of free calcium ions showing carious activity). In some embodiments, fluid can be used with the treatment instrument 1 disclosed herein to first clean and/or disinfect areas around the caries. In some embodiments, any suitable treatment fluid(s), including diluted bleach, chlorhexidine (CHX), peroxide, etc., can be used to dissolve and remove food residue, bacteria film, bacteria, and their metabolic productions. In some embodiments, other fluids (such as EDTA, or any buffers) can also be used to remove all or substantially all the substances around the carious region, whether extraneous to or immanent but separated from the main sound tooth structure, such as calcium ions, phosphate ions, etc.

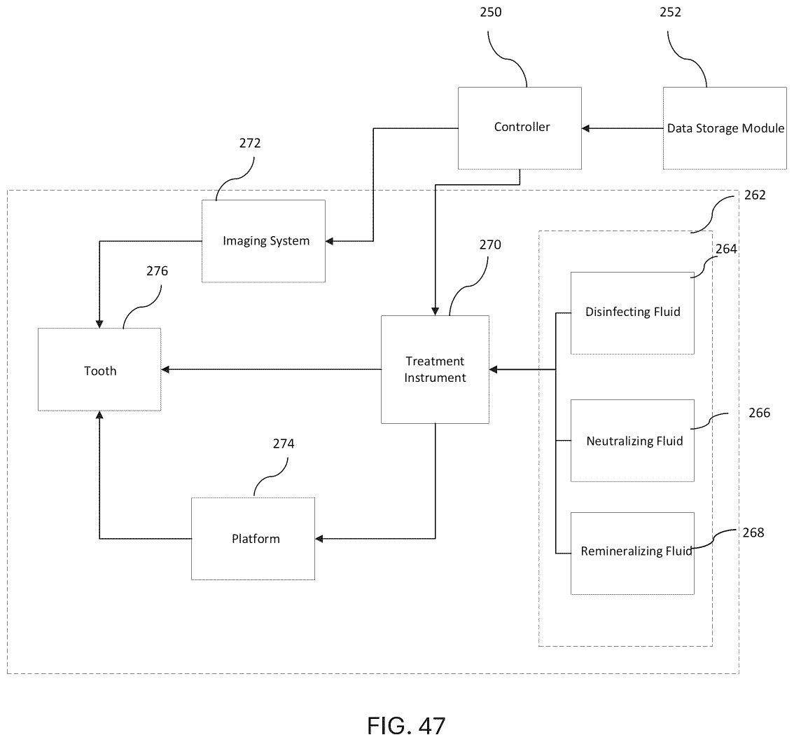

In some embodiments, a platform according to this disclosure can be built, if desirable, on the tooth to aid the treatment instrument 1 for cleaning and sanitization during the disinfecting process as illustrated in FIG. 47. Examples of the platform can be found throughout U.S. Pat. No. 10,722,325, filed May 1, 2014, which is incorporated by reference herein in its entirety and for all purposes.

In some embodiments, the treatment instrument 1 can deliver a disinfecting fluid or solution (e.g., water, ozonated water, sodium hypochlorite (bleach), peroxide solution, chlorhexidine, Listerine®, Ethylenediaminetetraacetic acid (EDTA)) for a desirable period of time (e.g., between 45 seconds to 10 minutes, between 45 seconds to 8 minutes, between 45 seconds to 5 minutes, between 45 seconds to 4 minutes, between 45 seconds to 2 minutes, between 3-10 minutes, between 4-6 minutes, for example, about 45 seconds, about 1 minute, about 2 minutes, about 4 minutes, about 5 minutes, about 6 minutes, about 10 minutes, about 15 minutes, or about 20 minutes) to disinfect carious lesions the include various types of bacteria, e.g., Streptococcus mutans and Lactobacillus, two bacteria that may be involved in the caries process. The chemical properties of the sodium hypochlorite along with the broadband pressure waves and fluid dynamic properties of the treatment instrument 1 may allow for penetration of the treatment fluid into the porous anatomy of the carious lesion and expedite disinfection and the reduction of bacteria.

In some embodiments, the treatment instrument 1 can deliver fluorohexane as a disinfecting fluid in order to remove the various types of bacteria that may be involved present at the carious region. The chemical properties of fluorohexane can allow for penetration of the fluid into the porous anatomy of the carious legion and lead to disinfection of bacteria, similar to sodium hypochlorite.

In other embodiments, disinfection may be achieved by utilizing laser irradiation. For example, as explained in connection with FIG. 1B, the pressure wave generator 10 can comprise a laser device. For example, the pressure wave generator 10 can comprise an optical fiber exposed to a chamber of the device 1. The laser device can pulse laser light along the fiber and into the treatment region to generate pressure waves in the fluid. Additional examples of pressure wave generators comprising lasers can be found throughout U.S. Pat. No. 9,675,426, which is incorporated by reference herein in its entirety and for all purposes.

The process of treating dental caries disclosed herein can further include neutralizing an acidity of the tooth. In cavitated lesions, bacteria can invade the dentin, resulting in increased acidity at the carious region. In incipient lesions, although bacteria is not necessarily present on the tooth, the tooth has increased acidity as a result of intrinsic or extrinsic acids which can occur from accumulated biofilm. The bacteria located on or around the incipient lesions can consume sugar, which in turn produces acid (or carbon dioxide gas). Therefore, it can be advantageous to neutralize the acidity of an incipient lesion. The process of tooth structure dissolution, separation, and decrement is defined as demineralization. Demineralization can be initiated usually because the pH value is usually lower than 7 at the caries section due to the acid generated by metabolic activity of local bacteria. Healthy tooth structure can be dissolved and thus damaged when the local pH drops lower than 5.5. As a result, tooth structure with a low pH value may be gradually separated from the main part and lost to the oral cavity environment. Therefore, fluids with relatively high pH values, or those capable of buffering pH, could be delivered to the caries section during the caries treatment process disclosed herein to neutralize the local tooth environment. For example, in some embodiments, a solution (e.g., buffering solution) with pH around 7, or in the range of 5-8 (e.g., pH of healthy saliva) can be delivered to the caries section to prevent demineralization. In some embodiments, a buffering solution with pH around 6 can be delivered to the caries region to prevent demineralization. Additionally, the buffering solution can have a pH in a range of 5-8.5, 5-8, 5-7.5, 6-8.5, 6.5-8, 6-7.5, 6.5-7.5, 6.5-7, or 6.8-7.2.

The demineralization process of a carious region can be based on a large presence or concentration of free calcium ions in tooth enamel pores, which can indicate demineralization is active and treatment may be beneficial using the systems and methods disclosed herein. Disinfection with sodium hypochlorite can reduce bacteria and begin to neutralize the acidity present at the carious region. Furthermore, although in incipient lesions there is not necessarily a significant (e.g., large) presence of bacteria, disinfecting the incipient lesions with sodium hypochlorite can remove any (e.g., small) amounts of bacteria present due to biofilms and the sodium hypochlorite may also begin to neutralize the acidity present in the tooth environment (e.g., incipient lesions).

In some embodiments, an alkaline fluid can be used during the disinfection process to react with acids in the carious lesions, resulting in a neutral or adjusted pH within the carious lesion. In some embodiments, for example, sodium hypochlorite can be used to neutralize or adjust pH within the carious lesion.

In another embodiments, the treatment instrument 1 can be switched to deliver, for example, a buffered saline solution (e.g., phosphate buffered saline (PBS)), or another buffering solution, for a desirable period of time (e.g., between 3-10 minutes, between 4-6 minutes, around 4 minutes, around 5 minutes, around 6 minutes, around 7 minutes, around 10 minutes, around 15 minutes, or around 20 minutes) and drive the buffering solution into the porous structure of the carious lesions to neutralize localized acid and facilitate active demineralization. The buffering solution can be biocompatible and can effectively neutralize the local tooth environment. The buffering solution can prevent the pH of the treatment region from changing significantly if an acid or base is added to the treatment region. For example, a buffering solution with pH around 7, or in the range of 5-8 (e.g., pH of healthy saliva) can be delivered to the caries region to prevent demineralization and neutralize the region. The buffering solution can have a pH of 7.4 or have a pH in a range of 7.1 to 7.7, in a range of 7.2-7.6, or in a range of 7.3 to 7.5. In some embodiments, the buffering solution can have a pH in a range of 5-8.5, 5-8, 5-7.5, 6-8.5, 6.5-8, 6-7.5, 6.5-7.5, 6.5-7, or 6.8-7.2. The buffering solution can cause the pH at the treatment region to be around 7+/−1, or 7+/−0.5. Furthermore, the buffering solution can cause the pH at the treatment region to be between 5-8.5, 5-8, 5-7.5, 6-8.5, 6.5-8, 6-7.5, 6.5-7.5, 6.5-7, 6.8-7.2 which can prevent bacteria or biofilms from being present at the treatment region.

One of skill in the relevant art would recognize that a buffering solution can prevent the acidity or alkalinity present at the treatment region from increasing over a period of time (e.g., after 1 day, 1 week, 2 weeks, 1 month, 2 months, etc.). In contrast, a typical alkaline fluid (e.g., sodium hypochlorite) may only be able to neutralize or adjust pH (e.g., to a pH value around 7) of the treatment region for a limited period of time (e.g., 1 hour, 4 hours, etc.) before the alkalinity or acidity of the treatment region begins to increase above a desired pH (e.g., pH above 8) or below a desired pH (e.g., a pH below 5). Additionally, other solutions (e.g., water, EDTA, peroxide solution, chlorhexidine) are not able to neutralize a treatment region or may only be able to neutralize or adjust pH (e.g., to a pH value around 7) of the treatment region for a limited period of time, as alternative solutions are unable to a resist pH change due to bacteria or biofilms. Accordingly, in some embodiments disclosed herein, the buffering solution is not water, EDTA, peroxide solution, chlorhexidine, or sodium hypochlorite, but rather, as explained above, a solution that prevents the pH of the treatment region from changing significantly (e.g., from changing by more than 5%, more than 10%, or more than 20%) if an acid or base is added to the treatment region after disinfecting. Introducing a buffering solution (e.g., PBS) to the treatment region (e.g., after delivery of an alkaline fluid) can advantageously neutralize the region, keep pH neutral (e.g., around 7) over a lengthy period of time, prevent the region from becoming acidic or alkaline, and inhibit present and future demineralization.

In some embodiments, the process of disinfecting the carious region 204 and neutralizing the acidity 206 of the carious region can include initially treating (e.g., disinfecting) the carious region with sodium hypochlorite and neutralizing the region with a phosphate buffered saline (PBS). The process of disinfecting the carious region 204 and neutralizing the acidity 206 of the carious region can happen simultaneously during the delivery of the treatment fluids (e.g., sodium hypochlorite, PBS) with the pressure wave generator 10. For example, although the disinfecting fluid (e.g., sodium hypochlorite) and buffering solution (e.g., PBS) can be delivered sequentially, the processes of disinfection and neutralization can overlap temporally. In some embodiments, the carious region can be disinfected (e.g., substantially cleaned of bacteria using a disinfecting solution delivered by pressure waves using the treatment instruments described herein), and the neutralization process (e.g., exposure to buffering solution with pressure waves using the treatment instruments described herein) can continue to neutralize the acidity of the region after disinfection. Additionally, the process of disinfecting and neutralizing the carious region can occur when sodium hypochlorite is delivered to the carious region and further neutralization of the carious region can occur when PBS is delivered to the carious region. The process of disinfection 204 and neutralization 206 can include a first step or treatment where the treatment instrument 1 can deliver a disinfecting solution (e.g., sodium hypochlorite) for a desirable period of time (e.g., about 5 minutes) to a treatment region. Additionally, the process of delivering sodium hypochlorite to a treatment region can be any desirable period of time that can effectively reduce or eliminate or destroy bacteria from the region (e.g., between 3-10 minutes, between 4-6 minutes, for example, about 4 minutes, about 5 minutes, about 6 minutes, about 10 minutes, about 15 minutes, or about 20 minutes). In cavitated lesions, the sodium hypochlorite can remove bacteria from the infected dentin (e.g., due to bacteria which can lead to demineralization and increased acidity in the carious region). Additionally, the sodium hypochlorite can immediately reduce or eliminate acidity present at the treatment site (e.g., cavitated lesion, carious region). In incipient lesions, the sodium hypochlorite can remove bacteria or biofilm present in the carious region, which can immediately (e.g., immediately post-treatment, 30 minutes post-treatment, 2 hours post-treatment) reduce or eliminate the acidity (e.g., due free calcium ions) present at the treatment site. The disinfecting solution (e.g., sodium hypochlorite) can cause the pH of the treatment region to be around 7, post-treatment. Additionally, the disinfecting solution can cause the pH of the treatment region to be between 5-8.5, 5-8, 5-7.5, 6-8.5, 6.5-8, 6-7.5, 6.5-7.5, 6.5-7, 6.8-7.2, post-treatment.

Following a step (e.g., first step) of delivering a disinfecting solution (e.g., sodium hypochlorite) to the treatment region, an additional (or second) step of delivering a buffering solution for a desirable period of time (e.g., between 45 seconds to 10 minutes, between 45 seconds to 8 minutes, between 45 seconds to 5 minutes, between 45 seconds to 4 minutes, between 45 seconds to 2 minutes, between 3-10 minutes, between 4-6 minutes, for example, about 45 seconds, about 1 minute, about 2 minutes, about 4 minutes, about 5 minutes, about 6 minutes, about 10 minutes, about 15 minutes, or about 20 minutes) to the treatment region can neutralize or further neutralize the carious region (e.g., cavitated lesions, incipient lesions) by arresting (e.g., preventing) the presence of free calcium ions. The buffering solution can be phosphate buffered saline (PBS). The process of delivering PBS (or another buffering solution) for a desirable period of time that can effectively reduce or eliminate acidity at the treatment region can be any desirable period of time (e.g., 3-10 minutes, between 4-6 minutes, around 4 minutes, around 5 minutes, or around 6 minutes, around 10 minutes, around 15 minutes, or around 20 minutes). The PBS can be delivered to the treatment region after (e.g., immediately after) the sodium hypochlorite is delivered. Additionally, a short period of time (e.g., about 1 minute, about 2 minutes, about 3 minutes, about 10 minutes, about 15 minutes) may pass between delivering sodium hypochlorite and delivering PBS. The carious region treated with sodium hypochlorite followed by PBS can eliminate essentially all acidity (e.g., the presence of free calcium ions, have a pH around 7 or neutral) immediately following treatment. Furthermore, the treatment with sodium hypochlorite followed by PBS can prevent acidity and/or bacteria from reforming or redeveloping (e.g., sustain arrest or presence of active carious legions) at the treatment region for long periods of time (e.g., about 10 days later, about 20 days later, about 30 days later, about 40 days later, 2 months later, 3 months later, etc.). The process of delivering a buffering solution (e.g., PBS) after a disinfecting solution can cause the pH of the treatment region to be around 7 post-treatment and long after treatment. Additionally, the process of delivering a buffering solution (e.g., PBS) after a disinfecting solution can cause the pH of the treatment region to be between 5-8.5, 5-8, 5-7.5, 6-8.5, 6.5-8, 6-7.5, 6.5-7.5, 6.5-7, 6.8-7.2, post-treatment.

The process of delivering a disinfecting solution (e.g., sodium hypochlorite) followed by a buffering solution (e.g., PBS) can be repeated for as many treatment cycles as desired (e.g., 2 treatment cycles, 3 treatment cycles) to sustain arrest and the presence of active carious regions. The disinfecting and neutralization process can also include first delivering a buffering solution (e.g., PBS), delivering a disinfecting solution (e.g., sodium hypochlorite) second, and ending the process with the delivery of the buffering solution (e.g., PBS) a second time. In some embodiments, the disinfecting and neutralization process is completed once the buffering solution is delivered to the treatment region for a desired period of time. Additionally, the disinfecting solution and/or buffering solutions used during this process can include any treatment fluid capable of disinfecting (e.g., water, ozonated water peroxide solution, chlorhexidine, Listerine®) or neutralizing carious regions by buffering (e.g., PBS). In comparison, treatment of a carious region with brushing alone may lead to bacteria and acidity being present after treatment, followed by an increased presence of acidity (e.g., free calcium ion) long after treatment (e.g., about 10 days later, about 20 days later, about 30 days later, about 40 days later). Additionally, treatment with only sodium hypochlorite may reduce bacteria and acidity at the treatment site immediately following treatment, but the presence of acidity (e.g., free calcium ion) may be present again long after treatment.

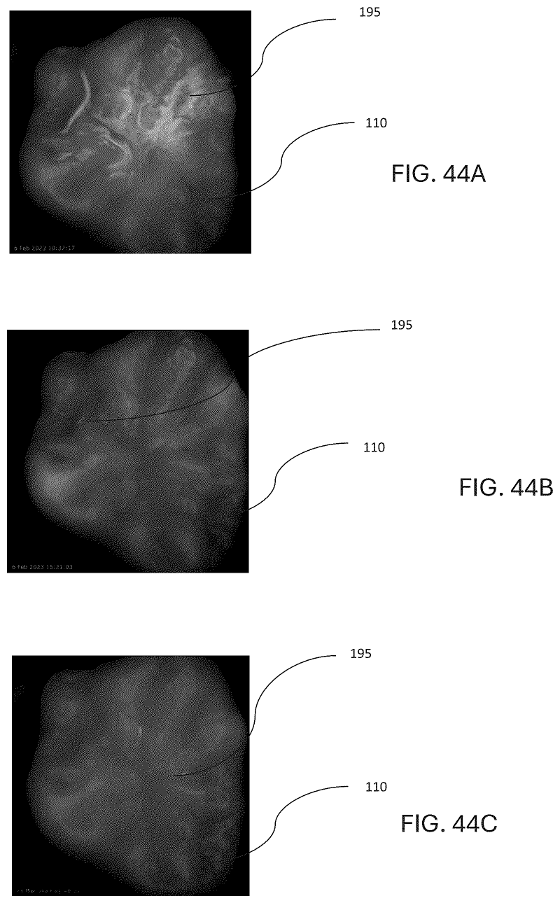

FIGS. 44A-44C show treatment or the process of disinfecting the carious region and neutralizing the acidity of the carious region with sodium hypochlorite and a phosphate buffered saline (PBS), respectively. FIG. 44A shows the tooth 110 with a presence of free calcium ions 195, before treatment (e.g., disinfection and neutralization) with sodium hypochlorite followed by PBS. The free calcium ions 195 (e.g., acidity due to free ions) present on the tooth 110 demonstrate carious legion activity due to the presence of free ions based on accumulated bacteria or biofilm.

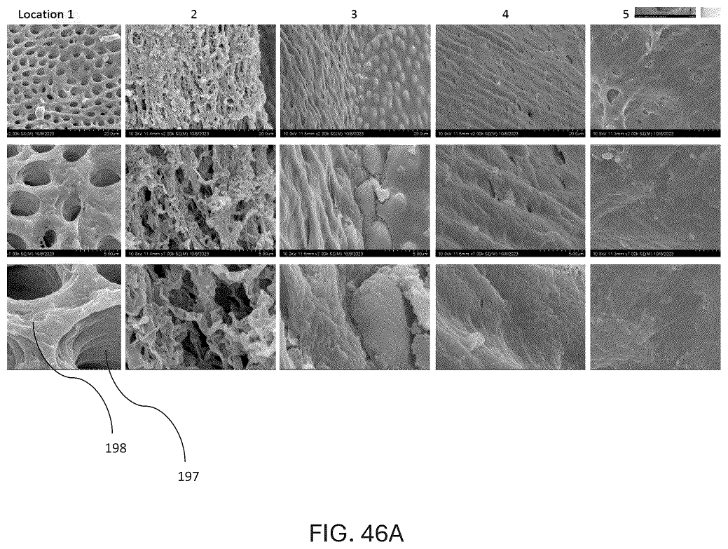

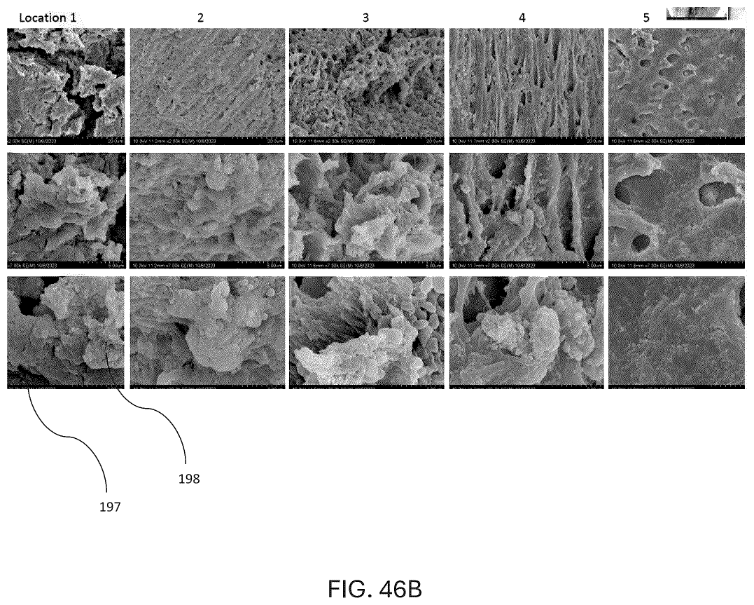

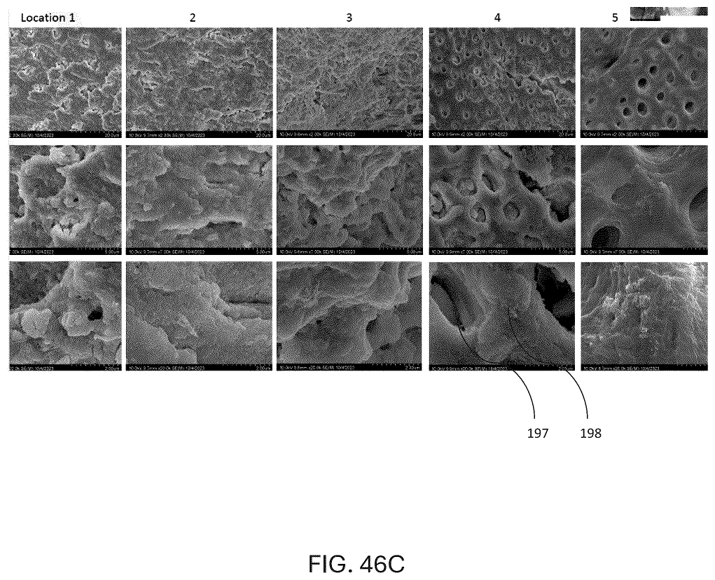

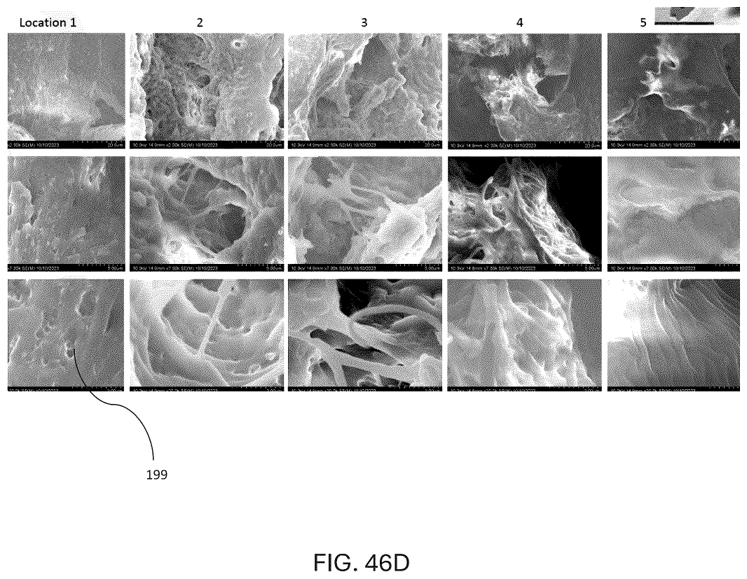

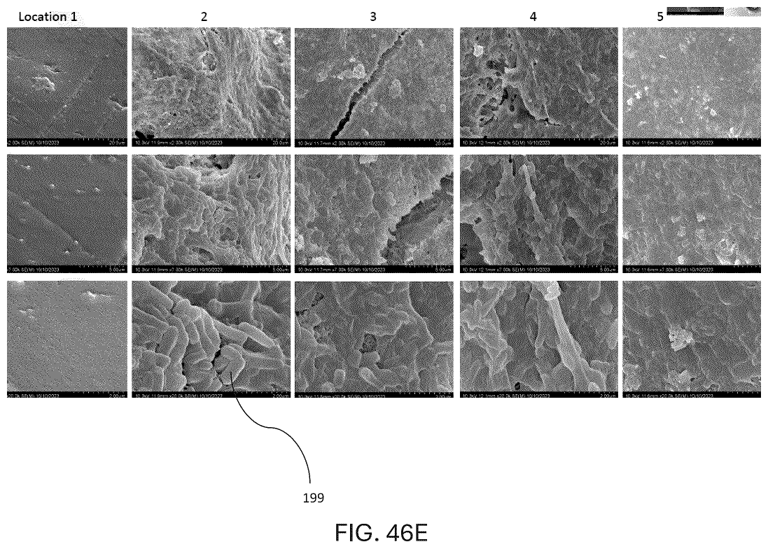

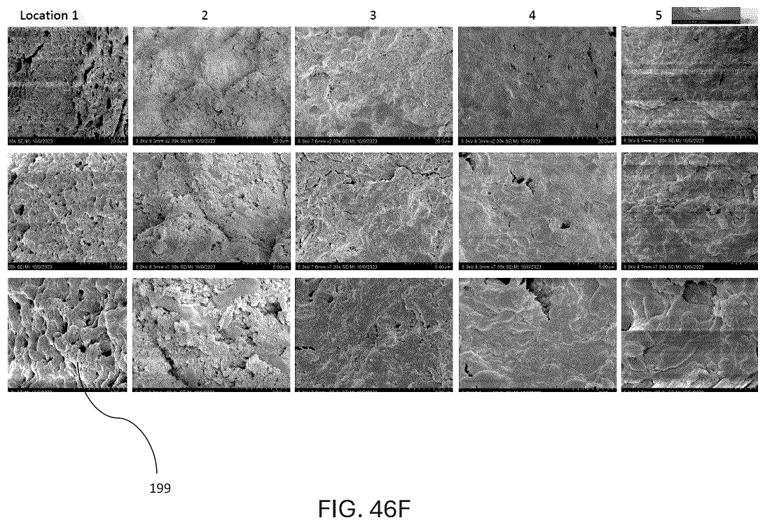

FIG. 44B shows the tooth 110 immediately after treatment (e.g., disinfection and neutralization) with sodium hypochlorite followed by PBS. FIG. 44B can represent the presence of free calcium ions 195 immediately, or within 1 minute, 5 minutes, 30 minutes, 1 hour, or about 2 hours following treatment. As shown in FIG. 44B, the tooth 110 has little to no free calcium ions 195 present on the tooth 110. The reduction or elimination of free calcium ions 195 can indicate that bacteria or biofilm present on the tooth 110 has been removed. Refer to FIGS. 46A-46F, which show the reduction and elimination of bacteria and biofilm present on the tooth 110. Since the tooth 110 has little to no free calcium ions 195 visible, this demonstrates or suggests that the process of applying sodium hypochlorite followed by PBS, significantly or completely eliminates or destroys bacteria and acidity present (e.g., due to the free calcium ions 195) on the tooth 110.

FIG. 44C shows the tooth 110 significantly after treatment on the tooth 110. For example, FIG. 44C can represent the presence of free calcium ions 195 about 35 days following treatment (e.g., disinfection and neutralization). Additionally, FIG. 44C can represent the presence of free calcium ions 195 about 10 days, about 20 days, about 30 days, about 40 days, about 2 months, and/or about 3 months following treatment. The tooth 110 has little to no free calcium ions 195 (e.g., acidity) present on the tooth following treatment. The reduction or elimination of free calcium ions 195 can indicate that bacteria or biofilm present on the tooth 110 has not generated during the post-treatment timeframe. Refer to FIGS. 46A-46F, which show the lack of bacteria and biofilm present on the tooth 110. Treatment with sodium hypochlorite and PBS can prevent the free calcium ions 195 from being generated (e.g., following treatment), demonstrating the treatment is effective at reducing or eliminating bacteria and acidity for long periods of time.

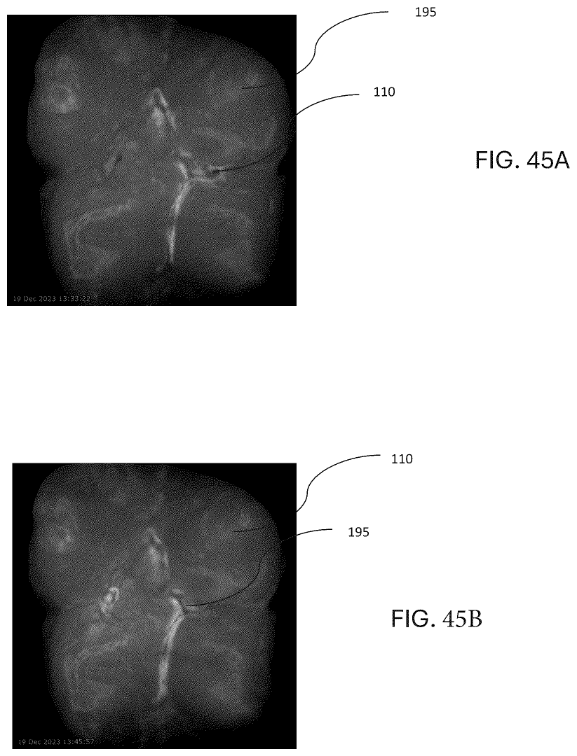

FIGS. 45A-45B show the treatment or process of disinfecting the carious region with a control group (e.g., with standard brushing to clean and 10 minutes of air drying). In FIG. 45A there is a large presence of free calcium ions 195 on the tooth 110 before treatment. FIG. 45B shows that there is still a large amount or prevalence of free calcium ions 195 present on the tooth 110 after treatment (e.g., brushing). Advantageously, the process shown in FIGS. 44A-44C represents that the process of treating a carious region with sodium hypochlorite followed by PBS is more effective at sustaining the absence of acidity and bacteria in the region as compared to the control group in FIGS. 45A-45B. Furthermore, disinfecting the carious region with the treatment instrument 1 by propagating pressure waves with a disinfecting fluid (e.g., sodium hypochlorite) can lead to a greater reduction of free calcium ions 195, bacteria, and biofilms compared to the control group. Additionally, neutralizing the carious region by propagating pressure waves with the buffering solution (e.g., PBS) can lead to a greater reduction and/or greater sustained reduction of free calcium ions present on the tooth 110 over a period of time compared to the control group. Incorporating a buffering solution (e.g., PBS) into the treatment process 200 can greatly improve treatment of incipient lesions and cavitated lesions (e.g., the carious region).

FIGS. 46A-46F illustrate the presence of biofilm and bacteria present on a tooth with treatment (e.g., sodium hypochlorite and PBS) and without treatment examined with a scanning electron microscope (SEM). FIG. 46A illustrates an anterior tooth treated with sodium hypochlorite and PBS. FIG. 46B illustrates a premolar tooth after treatment with sodium hypochlorite and PBS. FIG. 46C illustrates a molar after treatment with sodium hypochlorite and PBS. As can be seen in FIGS. 46A-46C, following treatment, the tooth structure is generally free of bacteria and biofilm (e.g., evidence of tubes and a non-flowy appearance). The dentinal tubules 197 are opened following the treatment. Additionally, globular protrusions 198 are observable around the dentinal tubules 197. Furthermore, irregular and dentinal surface are covered with an amorphic porous layer.

FIG. 46D illustrates an anterior tooth prior to treatment (e.g., or untreated) with sodium hypochlorite followed by PBS. FIG. 46E illustrates a premolar prior to treatment (e.g., or untreated) with sodium hypochlorite followed by PBS. FIG. 46F illustrates a molar prior to treatment (e.g., or untreated) with sodium hypochlorite followed by PBS. Biofilm or bacteria 199 is present on the tooth structure (e.g., evidence of bacteria nodule or a flowy appearance). Demineralized dentin is exposed on the on the carious lesions. Dentinal tubules 197 are not visible because they are obturated by demineralized dentin and biofilm or bacteria 199.

The process of treating dental caries disclosed herein can also include remineralization 208 of the tooth. At the carious region where tooth structure is partly lost due to demineralization, compounds (e.g., calcium compounds, such as fluorapatite) may be precipitated from oral saliva and deposited on the tooth structure when the compounds are oversaturated due to changes of pH value, temperature, etc. in the local environment. As a result, the tooth decay may also be recovered to its original health state during the precipitation and deposition processes. This process may be called remineralization.

In some embodiments, remineralization can include delivering fluids to the caries section to modify the local environment, initiating and/or maintaining the remineralization. In incipient lesions, the demineralization can be arrested (e.g., stopped) due to the procedures explained above (e.g., sodium hypochlorite and PBS), however, remineralization of the affected area does not naturally occur. Various fluids can be used to change different conditions of the local environment. In some embodiments, for example, a fluid can be used to provide fluoride ions and/or calcium ions. In some embodiments, a fluid (e.g., buffers, alkaline solutions, etc.) can be used to increase the local pH value. In some embodiments, PBS can be used to buffer or neutralize the pH of the carious lesion and, at the same time, provide a source of phosphate ions to facilitate remineralization. Phosphate ions may promote the formation of fluorapatite on teeth when provided in conjunction with fluoride ions. In some embodiments, EDTA can be used for remineralization. In some embodiments, hydroxyapatite solutions or strontium are applied to the affected region during remineralization.

The treatment instrument 1, in accordance with various embodiments, may be switched to deliver a fluoride-rich fluid (e.g., or any suitable fluid) for a desirable period of time (e.g., between 3-10 minutes, between 4-6 minutes, around 4 minutes, around 5 minutes, or around 6 minutes) to provide minerals to the carious lesions to allow for the start of the remineralization process. For example, the fluoride-rich fluid may include but are not limited to acidulated phosphate fluoride (APF) or sodium fluoride.

An aspect of this disclosure can also include creating a sealed environment 214 when delivering a fluid to a tooth using the treatment device 1. In such arrangements, the fluid may contact the treatment region of the tooth where the treatment is intended (e.g., the carious region on the exterior surface of the tooth). In some embodiments, when the sealed environment is created, the treatment device 1 may deliver any desirable fluids, including but not limited to any disinfecting, neutralizing, or remineralizing fluids disclosed above, to only the treatment region without spilling into the mouth and/or contacting the gum. With a sealed environment for delivering fluid, a wide variety of chemicals can be used, for example, a more aggressive neutralizing fluid, to treat caries without hurting the gum and/or other parts of the mouth. As explained herein, the treatment device 1 can comprise a fluid platform 2 comprising a chamber 6 to seal against the tooth to retain fluid such that the fluid can interact with the treatment region (e.g., the carious region). As explained herein, fluid can be controllably removed by way of suction port(s), which may be vented to ambient air.

In various embodiments, as shown in FIG. 43, treating dental caries can include, optionally, first assessing or imaging 202 the condition and status of a tooth before any one or more of the previous steps (e.g., cleaning, neutralizing, and/or remineralizing the tooth). Various caries detection methods or medical devices can be used to assess a condition and status of a tooth. For example, in some embodiments, assessing condition and status of the tooth can include using a visual tactic, X-ray, a fluorescence caries detection device, a luminescence imaging system, or any other imaging device or system, etc. to evaluate the existence and/or condition of the caries on the exterior surface of the tooth. The assessment can include evaluating any condition related to dental caries, including existence/quantity of bacteria, bacteria's metabolic products, calcium ions, food residue, phosphate ions, deteriorated saliva, porous tooth structure, exposed dentine/pulp tissue, etc. If one or more dental caries is detected, it can be treated by a treatment instrument 1 according to this disclosure (e.g., if pulp tissue is not exposed).

In some embodiments, an intra oral camera may be used to detect carious lesions (or caries) utilizing autofluorescence. The intra oral camera may uses light of different colors to detect presence of caries. In some embodiments, different colors or shades of light can be used to detect caries at different levels of disease. In some embodiments, for example, the intra oral camera can utilize visible blue light or ultraviolet light to detect caries, wherein healthy tooth structure may be viewed as green while regions of carious lesion may appear red.

In some embodiments, a device capable of assessing activity states of carious lesions may be utilized to determine location of lesions. In some embodiments, for example, the intra oral camera can detect bioluminescence such as blue light when the caries is active that the calcium ions from the active demineralization process react with some biochemical reagents, such as some photoprotein that exist widely inside the body of aquatic creatures (see FIGS. 44A-45B). The intra oral camera may be capable of capturing a grayscale image of the tooth surface, and the resultant bioluminescence, which is transformed into a colored image according to the bioluminescence level. The images can be combined into a hybrid image which can indicate the location of active caries images due to the calcium ions on the tooth surface. The color spectrum ranges from black or dark blue indicating no or low level caries activity (e.g., no or low calcium ions due to inactive demineralization) to orange/red for low levels of caries activity and to white which indicate high levels of active caries activity (e.g., high levels of demineralization)

In another embodiment, dyes or particles capable of identifying porosity of carious lesions may be utilized to determine location of lesions.

Another aspect of the process of treating dental caries may include reassessing or imaging 210 the conditions of the tooth. The condition and status of the tooth can be assessed again after the tooth has been treated with the treatment instrument 1. Any caries detection method or medical devices (such as visual tactic, X-ray, a fluorescence caries detection device, a luminescence imaging system, or any other imaging devices, etc.) can be used to evaluate any condition related to the caries, including existence and/or quantity of bacteria and/or its metabolic products, calcium ions, food residue, phosphate ions, deteriorated saliva, etc. One purpose of the reassessment is for comparison of the caries conditions before and after the treatment.

In some embodiments, assessment of the carious lesion can be performed using visible blue or ultraviolet light to demonstrate acute disinfection of the carious lesion. In some embodiments, assessment of the carious lesion may be performed using a device capable of detecting activity level of the lesion to demonstrate capability of the treatment instrument 1 to arrest progression of the demineralization cycle.

The process of treating dental caries disclosed herein can further include sealing and/or remineralization of the tooth to preserve the treated carious region and prevent further tooth deterioration. Sealing of the tooth may be achieved by applying sealing materials to seal the caries section to prevent further intrusion of extraneous substances, such as saliva, food residue, bacteria, etc. In some embodiments, any desirable sealing materials can be used, for example, composite resin dental sealants, glass ionomer dental sealants, amalgam dental sealant, etc.

In some embodiments, the cleaned carious region may also be sealed using varnish or gels. In some embodiments, varnish or gels containing fluoride can be used to seal and also induce remineralization on the caries section. A fluoride-rich varnish may be applied to the disinfected carious lesion to prevent bacteria from re-establishing within the porous tissue and to provide a mineral source to support continued remineralization. In other embodiments, a silver diamine fluoride (SDF) solution may also be applied to the carious lesion to support remineralization.

Another aspect of the disclosed process of treating caries is conducting long-term assessment. After a relatively longer time period post-treatment, the condition of the caries may be assessed or imaged 218 again following the above-mentioned treatment as a follow-up evaluation. Any caries detection method or medical devices (such as visual tactic, X-ray, a fluorescence caries detection device, a luminescence imaging system, or any other imaging devices, etc.) can be used to assess the treated caries. Reassessment can be performed to verify if the after-treatment caries condition is maintained or reversed for a satisfactorily long time.

FIG. 47 shows an exemplary system 200 where an imaging system 272 and a treatment instrument 270 can be used on a tooth 276, respectively, to treat dental caries. The treatment instrument 270 can correspond to any treatment system disclosed herein. The treatment instrument 270 can receive fluids 262, which can include a disinfecting fluid 264, a neutralizing fluid 266, and/or a remineralizing fluid 268. The treatment fluids can correspond to any treatment fluid disclosed herein. The treatment instrument 270 can deliver one or more of the fluids 262 from the treatment instrument 270 and to the tooth 276 or the platform 274. The platform 274 can correspond to any platform disclosed herein. The platform 274 can seal against the tooth to retain any of the fluids 262 delivered to the treatment instrument 270 such that the treatment fluid can interact (e.g., propagate throughout) with the treatment region. Furthermore, an imaging system can be used to detect a condition of a carious region of the tooth. To operate the system, a controller 250 can be used to perform certain functions of the system. Additionally, a data storage module 252 may have programming instructions which can dictate the systems operational features. The data storage module 213 may have read-only memory for the process to execute programmed functions. The data storage module 213 may also have writeable memory to store various programmed features. The data storage module 213 does not need to have both read-only memory and writeable memory.

The controller 250 (which can include processing electronics including a processing unit with logic) may be used to execute and deliver the programming instructions to the treatment instrument 270 in order to deliver a particular amount and type of treatment fluid 262 (e.g., disinfecting fluid, neutralizing fluid, remineralizing fluid) to the tooth 276 or platform 274. The controller 250 may deliver a signal to the imaging system 272 in order to image the carious region of the tooth. Additionally, the controller 250 can power (e.g., turn on and/or off) or modulate the flow of the treatment fluid 262 with one or more valves, conduits, or fluid lines to the treatment instrument 270. One or more valves controlled by the controller 250 can be positioned along on or more conduits 104, along an inlet line, and/or along an outline line 4. The controller 250 can activate a pressure wave generator 10 by activating a pump which can pressurize the treatment fluid 262 in order to deliver the treatment liquid 262 to the handpiece, along the one or more conduits 104 (e.g., which can have one or more valves positioned to control the flow of the treatment liquid 262), and to the tooth 276. A system relating to FIG. 47 is described further below.

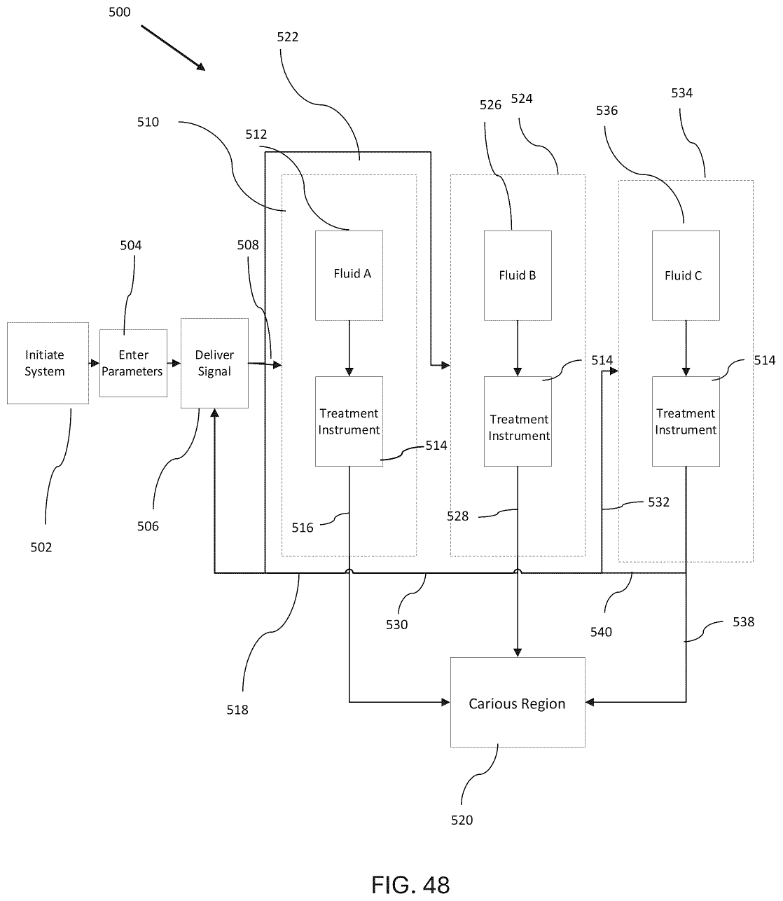

FIG. 48 depicts a schematic system diagram of an embodiment of a dental treatment system or treatment procedure 500. The treatment procedure 500 is similar to the treatment procedures disclosed above to remove carious region(s) from an exterior surface of the tooth.

As shown in FIG. 48, the treatment procedure 500 can be initiated at block 502, where the system is turned or started by a user. The controller 250 (shown in FIG. 47) can control the delivery or implementation of each of the blocks of the treatment procedure 500. For example, the controller 250 can control the delivery of the signals at block 506 in order to conduct the treatment procedure 500. The user can enter system procedures or steps at block 504 that are desirable for treatment of the tooth (e.g., disinfecting treatment, neutralizing, remineralization, etc.). Additionally, the user may specify different criteria (e.g., timing) for each treatment step or for the entire procedure. For example, the user may be able to enter or select the amount of time a fluid (e.g., sodium hypochlorite) is delivered to the tooth interface or carious region. Additionally, the user may be able to select an automated process or standard process, where each step of the treatment procedure 500 is fully automated to deliver fluid through the fluid platform or treatment instrument 1 to remove a carious region from a tooth. Furthermore, the treatment procedure 500 may be fully automatic, where a user begins treatment by starting (e.g., turning on, powering) the system, initiating the procedure on a user interface (of a console for example), and the remaining implementation steps of the treatment procedure 500 are controlled (e.g., by the controller). A user, in this case would manually hold the treatment instrument 1 against the carious region tooth 110 as the treatment procedure 500 is conducted and completed. In other embodiments, the user can attach or adhere the treatment instrument 1 against the tooth 110 or against a platform during the treatment procedure 500.

At block 506, the signal from 504 is received and relayed (e.g., due to controller 250). The signal can be delivered to any of blocks 510, 524, and 534 to initiate and deliver treatment. When the parameters entered or selected at block 504 require disinfecting, a signal is relayed 508 to block 510. At block 510, a fluid A 512 is delivered to the treatment instrument 514 (also known as treatment instrument 1). The treatment instrument 514 can deliver any treatment fluid disclosed herein (e.g., fluid A 512, fluid B 526, fluid C 536). The fluid A 512 can be any fluid (e.g., any suitable disinfecting liquid such as diluted bleach, chlorhexidine (CHX), peroxide, etc.) capable of disinfecting (e.g., removing or destroying bacteria and biofilm) from the tooth surface). A signal 516 is then relayed to deliver the fluid from the treatment instrument 514 to the carious region 520 (or any carious region disclosed previously herein). Once treatment is completed, a signal 518 can be relayed back to block 506 in order to continue treatment.

At block 524, the steps for neutralizing the affected tooth area are processed and delivered. A signal 522 is relayed from block 506 to initiate the process. A fluid B 526 can be delivered to the treatment instrument 514. The fluid B 526 can be any suitable buffering fluid (e.g., phosphate buffered saline (PBS), or another buffering solution) capable of neutralizing the acidity of the carious region. A signal 528 is then relayed which allows the fluid B 526 from the treatment instrument 514 to be delivered to the carious region 520. Once treatment is completed, a signal 530 can be relayed back to block 506 in order to continue treatment.

At block 534, the steps for demineralizing the affected tooth area are processed and delivered. A signal 532 is relayed from block 506 to initiate the process. A fluid C 536 can be delivered to the treatment instrument 514. The fluid C 536 can be any fluid (e.g., fluorapatite, EDTA, hydroxyapatite solutions, strontium solutions, etc.) capable of remineralizing the region. A signal 438 is then relayed with allows the fluid C 536 from the treatment instrument 514 to be delivered to the surface 420. Once the treatment is completed a signal 440 can be relayed back to block 506, which can indicate that the treatment procedure 500 is complete.

### Example Process of Treating a Tooth Using a Treatment Instrument, a Fluid Platform, a Matrix, and a Sealant

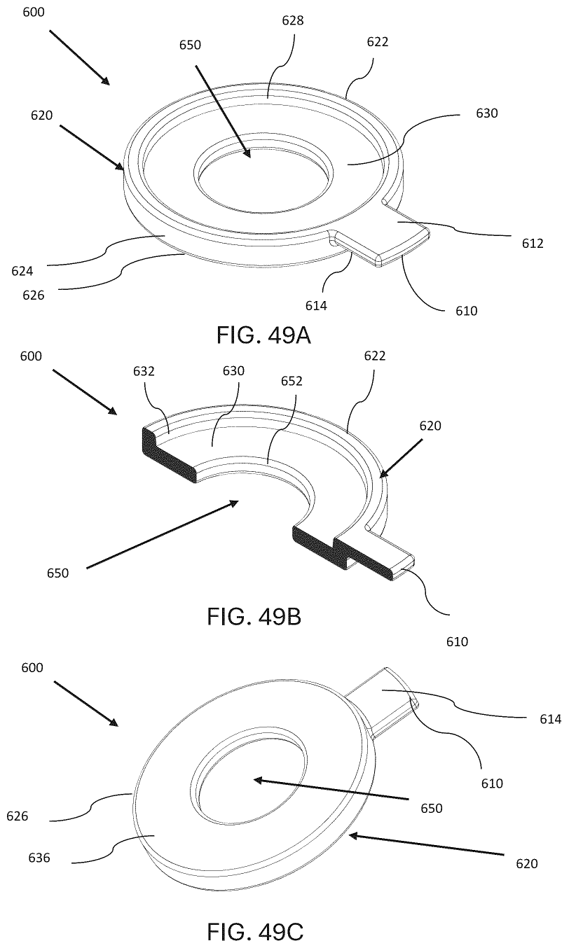

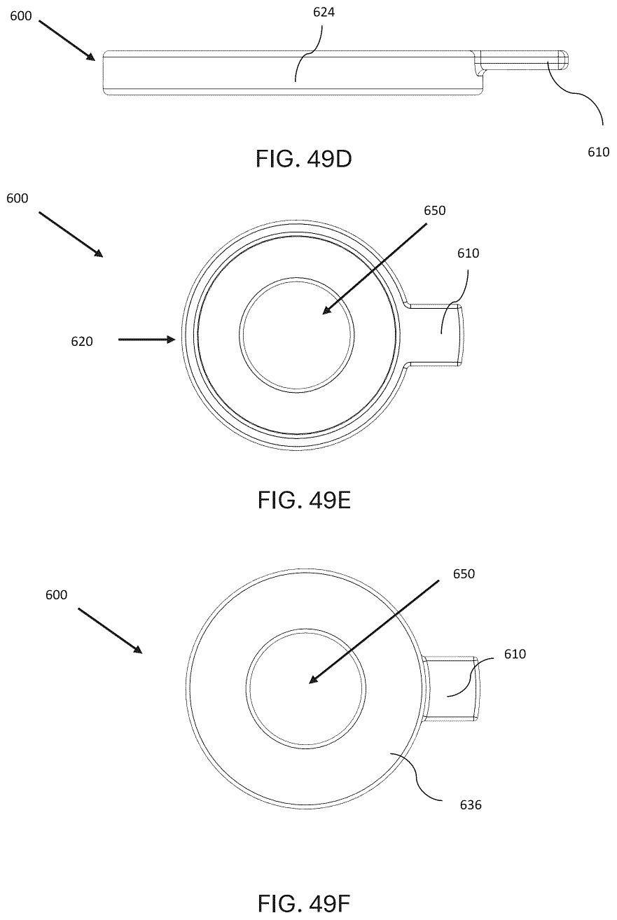

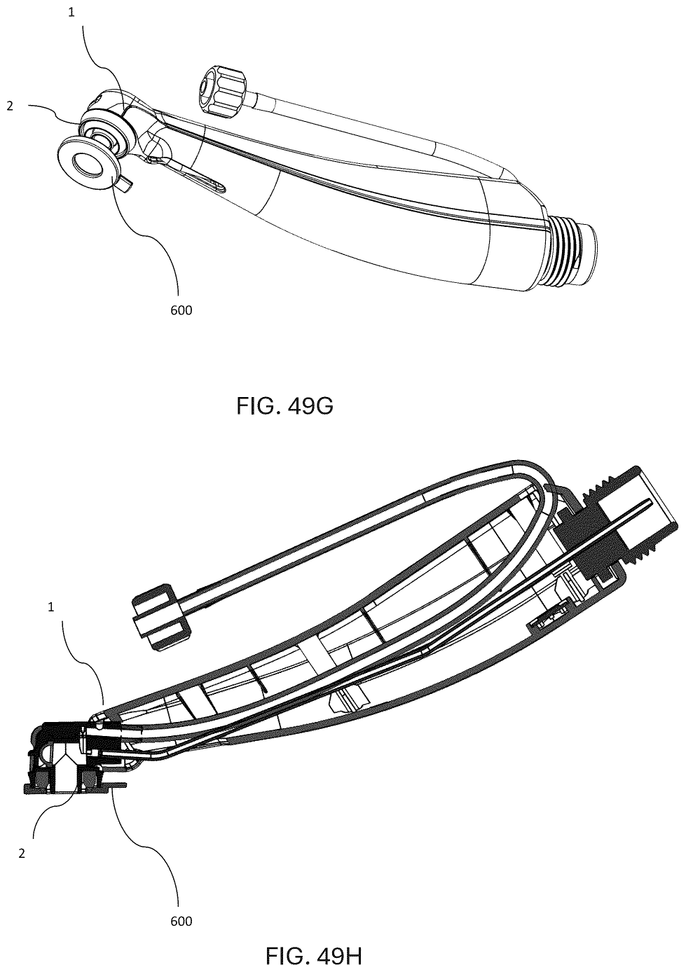

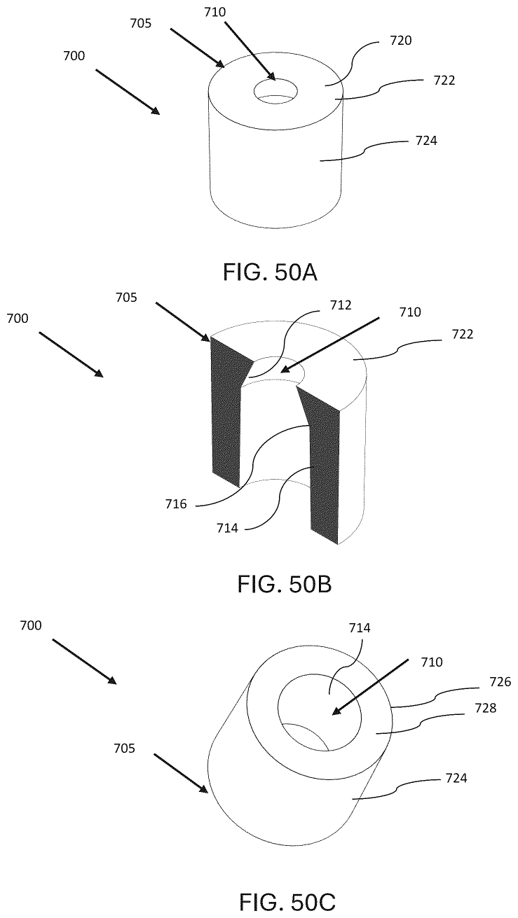

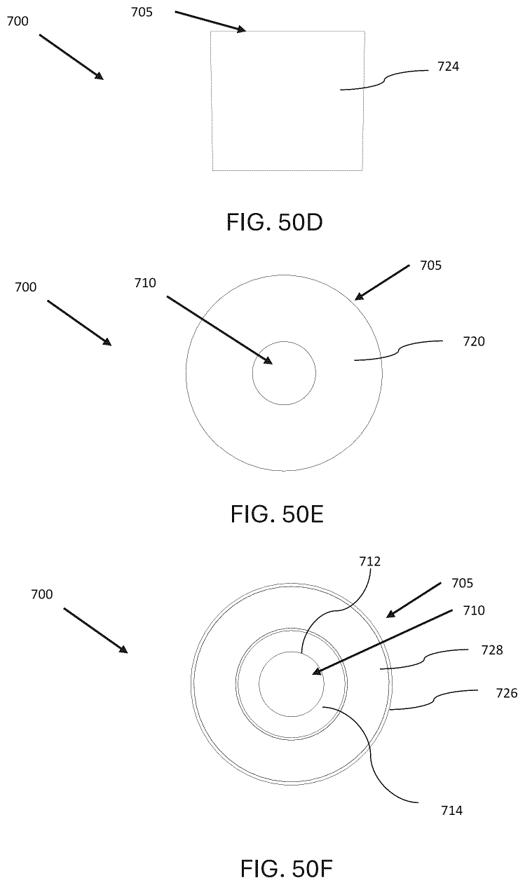

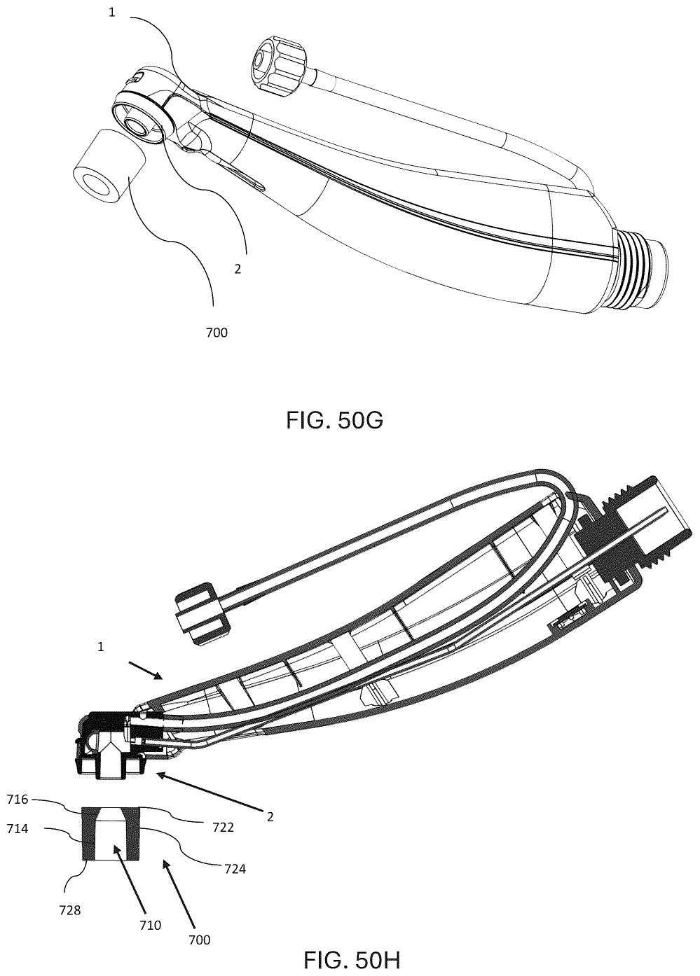

FIGS. 49A-50H disclose various embodiments related to a matrices 600 and 700. The matrices 600 and 700 can be used in conjunction with a sealant material or conforming material 400 as described herein to facilitate the cleaning and/or filling of a treatment region of a tooth 110. The matrices can create a sealed environment between the tooth 110 and the treatment platform to prevent treatment fluids or gases from escaping. Although the examples of 49A-50H are described in the context of treating carious regions on an exterior surface of the tooth, it should be appreciated that the systems and methods of FIGS. 49A-50H can additionally be used in endodontic treatments to disinfect and neutralize diseased root canals. In some embodiments, the matrix 600 can be an applicator used to apply the conforming material 400 to a tooth to form a platform 405 on the tooth, as described in further detail herein. In some embodiments, the matrix 600 can be a frame, scaffolding, or mold for formation of the platform 705 from the conforming material 400. In some embodiments, the matrix 600 can be used to form the platform 405 of conforming material 400 on the tooth without requiring a tooth cap or other hardware to be attached to the tooth. The platform 405 can be used to support a treatment instrument (e.g., to support a fluid platform 2 of a treatment instrument 1) during a treatment procedure.

FIG. 49A is a top perspective view, FIG. 49B is a cross sectional top perspective view, and FIG. 49C is a bottom perspective view of a matrix 600 according to some embodiments. FIG. 49D is a front view, FIG. 49E is a top view, FIG. 49F is a bottom view, of the matrix 600 shown in FIGS. 49A-49C. FIGS. 49G-H show the matrix connected to a treatment instrument 1. In some embodiments, the matrix 600 may include a handle 610, a rim 620, and an upper surface 630.

As shown in FIG. 49A, the matrix 600 can include a channel 650 in the form of a through hole that extends through the matrix 600 from a superior end to an inferior end (e.g., along the superior-inferior axis or central axis of the matrix 600). In some embodiments the channel 650 may have a constant cross-sectional area. In some embodiments the channel 650 may have a variable cross-sectional area, for example, with a cross-sectional area that increases in the superior direction. In some embodiments, the channel 650 may act as a vent channel or relief channel to prevent the buildup of pressure within the tooth, as discussed in further detail herein.

In some embodiments, the handle 610 can include a handle top 612 opposite a handle bottom 614. The handle 610 can be in the form of a generally longitudinal structure extending along the anterior-posterior axis. In some embodiments, an inferior end of the handle 610 may connect to an outer surface 624 of the rim 620.

In some embodiments, the rim 620 can include an upper edge 622 and a lower edge 626. The rim 620 can also include an inner wall 628. The upper edge 622 can be positioned level with the handle 610. In some embodiments, the rim 620 may be disc shaped or generally disc shaped. The rim 620 may have a circular cross-section in a plane formed by the right-left and anterior-posterior plane and have a height or thickness along the superior-inferior axis. The rim 620 or upper edge 622 may connect to the fluid platform 2 or the sealing cap 3. The rim 620 can be coupled to the main body 40 of the fluid platform. In some embodiments, a bottom cap 92 of the fluid platform 2 may be placed within the inner wall 628 and adjacent to the upper edge 622. The upper surface 630 may connect or form a seal with the sealing cap 3, and the channel 650 may be coupled or fluidically coupled to carious region of the tooth 110, and the treatment area of the tooth 110. Additionally, the channel 650 may be concentric with the chamber 6 or distal chamber 70 of the fluid platform 2.

In some embodiments, the rim 620 can include a lower surface 636. The lower surface 636 can be positioned below (inferior to or distal to) the upper edge 622 and the handle 610. The lower surface 636 can be disc shaped or generally disc shaped. The lower surface 636 may have a circular cross-section in a plane formed by the right-left and anterior-posterior plane and have a height or thickness along the superior-inferior axis. In some embodiments, the lower surface 636 can be concentric with the rim 620. The lower surface 636 may connect to the upper surface 630 via the outer surface 624 and the rim 620. In some embodiments, the lower surface 636 and rim 620 can be used to seal the matrix 600 against the tooth 110 and to deliver fluid to carious region of the tooth 110. The lower surface 636 can be coated with a sealant or any other biocompatible gel or adhesive capable of forming a platform.

In some embodiments, the shapes of the lower surface 636 and rim 620 can form corresponding shapes of the platform 405 of the conforming material 400. For example, in some embodiments, a conforming material 400 can be applied to the matrix 600 over the lower surface 636 and can adopt a corresponding shape. An example of conforming material 400 applied to the matrix 600 is shown in FIG. 42D. The matrix 600 can then be used to apply the shape conforming material 400 to a tooth to form the platform 405.

In embodiments of the matrix 600 with a channel 650, the channel 650 may serve as a relief channel for air and prevent the formation of voids within a conforming material 400 while curing. In some procedures, in the absence of vent pathways from the tooth, the application of a platform 405 may cause an increase in pressure within the tooth that creates voids within the conforming material 700. The channel 650 can allow for the release of pressure from the tooth without the formation of voids in the conforming material 400.

In some embodiments and as shown in FIGS. 49A-49C, the handle top 612 may be elongated along the anterior-posterior axis. In some embodiments, an elongated handle top 612 may facilitate handling of the matrix 600 by a clinician. In some embodiments, the handle 610 may comprise circumferential edge and/or other protuberances to facilitate grasping of the handle 610 by a clinician.

With reference to FIGS. 50A-50H, another embodiment of a matrix 700 is disclosed. The matrix 700 can be coupled to the fluid platform 2 and sealing cap 3 at platform 705. Additionally, the matrix 700 can extend from the sealing cap 2 to the tooth 110. The clinician may then place the matrix 700 on the tooth 110.

The matrix 700 may include features that are similar to the features of the matrix 600, such as an edge 722 (e.g., round, circular) that corresponds to the upper edge 622 of the matrix 600, a ridge surface 720 which corresponds to upper surface 630 of the matrix 600 and an access opening 710 that corresponds to the channel 650 of the matrix 600. The ridge wall 724 and the ridge surface 720 of the matrix 700 may engage the bottom cap 92 of the fluid platform 2. Additionally, the access opening 710 of the matrix 700 may be in fluidic communication with the the tooth 110 and/or carious region of the tooth 110. The access opening 710 may be in fluidic communication with the chamber 6 and/or distal chamber 70 and can form a passageway from the fluid platform 2 to the tooth 110. In some embodiments, the clinician may access the access opening 710 by reforming the access opening 710 increase the size of the access opening 710 and/or change the shape of the access opening 710 (e.g., to substantially match and/or form a smooth transition with the tooth 110).

The access opening 710 may include a first region 712 and a second region 714, where the second region 714 is positioned below or inferior to the 712. The first region 712 may taper outwards from the ridge surface 720 until it reaches an upper transition point 716 of the second region 714. The first region 712 may be coupled to an outer wall of the distal chamber 70. The first region 712 may conform (e.g., reshape) in order to be secured to the fluid platform 2. The diameter of the access opening 710 at the ridge surface 720 can be smaller than the diameter of the first region 712 at the upper transition point 716. Additionally, the diameter of the access opening 710 at the ridge surface 720 can be smaller than the diameter of the access opening 710 at the bottom surface 728. The second region 714 extends downwards (e.g., inferior) until it reaches a bottom surface 728 of the matrix 700. The second region 714 can have no taper and extend laterally downward to form a cylindrical channel. The second region 714 can engage an exterior surface of the tooth 110 and the carious region of the tooth 110. Additionally, the geometry (e.g., tapered first region 712 extending into the cylindrical second region 714) can optimize the fluid pathway from the treatment device 1 to the tooth 110. In some embodiments, the ridge wall 724 can taper from the ridge surface 720 or upper edge 722 to the bottom surface 728 or lower edge 726. Therefore, an outer diameter of the ridge surface 720 can be greater than an outer diameter of the bottom surface 728.

After the bottom surface bottom surface 728 or the access opening 710 has formed a seal on the tooth 110, the treatment instrument 1 can be introduced. For example, a fluid platform 2 of the treatment instrument 1 may be positioned on the ridge surface 720 and within the channel or access opening 710 of the matrix 700. The ridge surface 720 and access opening 710 may assist in locating the fluid platform 2 at the center of the platform. The conforming nature of the matrix 700 when connected to the fluid platform 2 may also restrict or prevent movement of the treatment instrument along the ridge surface 720 of the platform 2 (e.g., left-right and anterior posterior movement). For example, the engagement between the ridge wall 724 and the fluid platform 2 may prevent movement by more than 0.010 in.

Additionally, after engagement between the fluid platform 2 and the matrix 700, the clinician may begin the procedure. The clinician may ensure any conduits 104 and/or tubing is not kinked or restricted. The clinician may ready a console 102 of the system 100 and press down on a foot pedal of the console, which may control the delivery of procedure fluid. The procedure may be paused by releasing the foot pedal. While pressing down on the console's foot pedal, the clinician may ensure that the fluid platform 2 remains properly seated on the matrix 700 to retain the fluidic seal between the fluid platform 2, the access opening 710 of the matrix 700, and thus the treatment area of the tooth 110.

Although the example process as described relative to FIGS. 49A-50H are described in the context of treating carious regions on an exterior surface of the tooth, it should be appreciated that the systems and methods of FIGS. 49A-50H can additionally be used in endodontic treatments to disinfect and neutralize diseased root canals

Reference throughout this specification to “some embodiments” or “an embodiment” means that a particular feature, structure, element, act, or characteristic described in connection with the embodiment is included in at least one embodiment. Thus, appearances of the phrases “in some embodiments” or “in an embodiment” in various places throughout this specification are not necessarily all referring to the same embodiment and may refer to one or more of the same or different embodiments. Furthermore, the particular features, structures, elements, acts, or characteristics may be combined in any suitable manner (including differently than shown or described) in other embodiments. Further, in various embodiments, features, structures, elements, acts, or characteristics can be combined, merged, rearranged, reordered, or left out altogether. Thus, no single feature, structure, element, act, or characteristic or group of features, structures, elements, acts, or characteristics is necessary or required for each embodiment. All possible combinations and subcombinations are intended to fall within the scope of this disclosure.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment.

The foregoing description sets forth various example embodiments and other illustrative, but non-limiting, embodiments of the inventions disclosed herein. The description provides details regarding combinations, modes, and uses of the disclosed inventions. Other variations, combinations, modifications, equivalents, modes, uses, implementations, and/or applications of the disclosed features and aspects of the embodiments are also within the scope of this disclosure, including those that become apparent to those of skill in the art upon reading this specification. Additionally, certain objects and advantages of the inventions are described herein. It is to be understood that not necessarily all such objects or advantages may be achieved in any particular embodiment. Thus, for example, those skilled in the art will recognize that the inventions may be embodied or carried out in a manner that achieves or optimizes one advantage or group of advantages as taught herein without necessarily achieving other objects or advantages as may be taught or suggested herein. Also, in any method or process disclosed herein, the acts or operations making up the method or process may be performed in any suitable sequence and are not necessarily limited to any particular disclosed sequence.

1. A method of treating a carious region on an exterior surface of a tooth, the method comprising:
treating the carious region on the exterior surface of the tooth using a treatment instrument comprising a pressure wave generator, wherein treating the carious region on the tooth comprises:
propagating pressure waves through a treatment liquid with the pressure wave generator to reduce infection of the carious region;
neutralizing an acidity of at least the carious region with a buffering solution; and
remineralizing at least the carious region.

2. The method of claim 1, further comprising sealing and/or remineralizing the tooth.

3. The method of claim 1, further comprising imaging the tooth to evaluate an existence and/or condition of carious tissue in the carious region.

4. (canceled)

5. The method of claim 1, further comprising imaging the tooth before treating the carious region on the tooth.

6. (canceled)

7. The method of claim 1, further comprising imaging the tooth after sealing and/or remineralizing the tooth.

8. The method of claim 1, wherein disinfecting bacteria of at least the carious region comprises delivering an alkaline fluid to the carious region.

9. The method of claim 1, wherein remineralizing at least the carious region comprises delivering a fluid containing fluoride and/or calcium ions to the carious region.

10. The method of claim 1, wherein treating the carious region comprises positioning a fluid platform against the tooth over the carious region.

11. The method of claim 10, further comprising providing a fluid seal between the fluid platform and the tooth.

12. The method of claim 1, wherein the buffering solution comprises a phosphate buffered saline (PBS).

13. The method of claim 1, wherein treating the carious region includes delivering a disinfecting solution to destroy bacteria, wherein treating the carious region includes delivering the buffering solution after delivering the disinfecting solution to neutralize an acidity of the carious region, wherein the treatment liquid comprises the disinfecting solution.

14. The method of claim 13, wherein treating the carious region includes propagating pressure waves through a sodium hypochlorite treatment liquid to disinfect the carious region and, subsequently, propagating pressure waves through a buffering solution to neutralize the carious region, wherein treating the carious region includes significantly reducing a presence of free calcium ions at the carious region.

15. (canceled)

16. A method of treating a carious region on an exterior surface of a tooth, the method comprising:
imaging the tooth to detect caries;
after imaging, positioning a treatment instrument on the exterior surface of the tooth over the carious region;
activating a pressure wave generator of the treatment instrument to propagate pressure waves through a treatment fluid to at least partially disinfect the carious region; and
performing one or more of neutralizing the carious region of the tooth with a buffering solution or remineralizing the carious region of the tooth.

17. The method of claim 16, further comprising sealing and/or remineralizing the tooth.

18. The method of claim 16, wherein imaging the tooth comprising using an intra oral camera to detect the caries utilizing autofluorescence.

19. The method of claim 16, further comprising imaging the tooth after treating the caries on the tooth.

20. The method of claim 19, further comprising imaging the tooth after sealing and/or remineralizing the tooth.

21. The method of claim 16, wherein disinfecting the carious region of the tooth comprises delivering an alkaline fluid to the carious region.

22. The method of claim 16, wherein remineralizing the carious region of the tooth comprises delivering a fluid containing fluoride and/or calcium ions to the carious region.

23. The method of claim 16, wherein positioning the treatment instrument comprises positioning a fluid platform against the tooth such that a chamber of the fluid platform is disposed over the carious region.

24. The method of claim 23, further comprising providing a fluid seal between the fluid platform and the tooth.

25. The method of claim 16, wherein the buffering solution comprises a phosphate buffered saline (PBS).

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. A method of treating a carious region on an exterior surface of a tooth, the method comprising:
treating the carious region on the exterior surface of the tooth using a treatment instrument comprising a pressure wave generator, wherein treating the carious region on the tooth comprises:
delivering a disinfecting solution to destroy bacteria using pressure waves generated by the pressure wave generator; and
after delivering the disinfecting solution, delivering a buffering solution to neutralize an acidity of at least the carious region using pressure waves generated by the pressure wave generator.

69. The method of claim 68, wherein the pressure wave generator is configured to generate pressure waves with the disinfecting solution to disinfect the carious region when a chamber of the treatment instrument is substantially filled with the disinfecting solution.

70. The method of claim 68, wherein the pressure wave generator is configured to generate pressure waves with the buffering solution to neutralize the carious region when a chamber of the treatment instrument is substantially filled with the buffering solution.

71. The method of claim 68, wherein the buffering solution comprises a phosphate buffered saline (PBS).

72. The method of claim 68, wherein the disinfecting solution comprises sodium hypochlorite.

73. (canceled)

74. (canceled)

75. (canceled)

76. (canceled)

77. (canceled)

78. (canceled)
